(19) United States
(12) Patent Application Publication
(10) Pub. No.: US 2021/0322485 A1
(43) Pub. Date: Oct. 21, 2021

(54) MITOCHONDRIAL AUGMENTATION THERAPY WITH STEM CELLS ENRICHED WITH FUNCTIONAL MITOCHONDRIA

(71) Applicant: Minovia Therapeutics Ltd., Tirat Hacarmel (IL)

(72) Inventors: Natalie Yivgi Ohana, Haifa (IL); Uriel Halavee, Ramat Gan (IL); Shmuel Bukshpan, Ramat Hasharon (IL); Noa Sher, Haifa (IL)

(21) Appl. No.: 17/252,674

(22) PCT Filed: Jul. 22, 2019
(86) PCT No.: PCT/IL2019/050828

§ 371 Date: Dec. 15, 2020

Related U.S. Application Data
(60) Provisional application No. 62/701,783, filed on Jul. 22, 2018.

(51) Int. Cl. A61K 35/545; A61K 35/28; A61K 35/51; A61K 35/38; A61K 35/15; A61K 35/17; A61K 35/14; A61K 35/50; A61K 45/06; A61P 39/00; C12N 5/0775; C12N 5/073; C12N 5/078; C12N 5/071

(52) U.S. Cl. CPC A61K 35/545; C12N 5/0632; A61K 35/51; A61K 35/38; A61K 35/15; A61K 35/17; A61K 35/14; A61K 35/50; A61K 45/06; A61P 39/00; C12N 5/0663; C12N 5/0667; C12N 5/0605; C12N 5/0634; A61K 35/28

(57) ABSTRACT

The present invention provides stem cells enriched with healthy functional mitochondria, and therapeutic methods utilizing such cells for the alleviation of debilitating conditions, including aging, and age-related diseases as well as the debilitating effects of anti-cancer therapies in subjects in need thereof.

### FIELD OF THE INVENTION

The present invention relates to stem cells enriched with functional mitochondria, and therapeutic methods utilizing such cells to diminish the debilitating effects of various conditions, including aging and age-related diseases as well as the debilitating effects of anti-cancer therapy treatments.

### BACKGROUND OF THE INVENTION

The mitochondrion is a membrane bound organelle found in most eukaryotic cells, ranging from 0.5 to 1.0 μm in diameter. Mitochondria are found in nearly all eukaryotic cells and vary in number and location depending on the cell type. Mitochondria contain their own DNA (mtDNA) and their own machinery for synthesizing RNA and proteins. The mtDNA contains only 37 genes, thus most of the gene products in the mammalian body are encoded by nuclear DNA.

Mitochondria perform numerous essential tasks in the eukaryotic cell such as pyruvate oxidation, the Krebs cycle and metabolism of amino acids, fatty acids and steroids. However, the primary function of mitochondria is the generation of energy as adenosine triphosphate (ATP) by means of the electron-transport chain and the oxidative-phosphorylation system (the “respiratory chain”). Additional processes in which mitochondria are involved include heat production, storage of calcium ions, calcium signaling, programmed cell death (apoptosis) and cellular proliferation.

The ATP concentration inside the cell is typically 1-10 mM ATP can be produced by redox reactions using simple and complex sugars (carbohydrates) or lipids as an energy source. For complex fuels to be synthesized into ATP, they first need to be broken down into smaller, simpler molecules. Complex carbohydrates are hydrolyzed into simple sugars, such as glucose and fructose. Fats (triglycerides) are metabolized to give fatty acids and glycerol.

The overall process of oxidizing glucose to carbon dioxide is known as cellular respiration and can produce about 30 molecules of ATP from a single molecule of glucose. ATP can be produced by a number of distinct cellular processes. The three main pathways used to generate energy in eukaryotic organisms are glycolysis and the citric acid cycle/oxidative phosphorylation, both components of cellular respiration, and beta-oxidation. The majority of this ATP production by non-photosynthetic eukaryotes takes place in the mitochondria, which can make up nearly 25% of the total volume of a typical cell. Various mitochondrial disorders are known to result from defective genes in the mitochondrial DNA.

WO 2016/135723 to the present inventors discloses mammalian bone marrow cells enriched with mitochondria for treatment of mitochondrial diseases.

US 2012/0058091 discloses diagnostic and therapeutic treatments related to mitochondrial disorders. The method involves microinjecting heterologous mitochondria into an oocyte or embryonic cell wherein the heterologous mitochondria are capable of achieving at least normal levels of mitochondrial membrane potential in the oocyte or embryonic cell.

WO 2001/046401 discloses embryonic or stem-like cells produced by cross species nuclear transplantation. Nuclear transfer efficiency is enhanced by introduction of compatible cytoplasm or mitochondrial DNA (same species or similar to donor cell or nucleus).

WO 2013/002880 describes compositions and methods comprising bio-energetic agents for restoring the quality of aged oocytes, enhancing oogonial stem cells or improving derivatives thereof (e.g., cytoplasm or isolated mitochondria) for use in fertility-enhancing procedures.

US 20130022666 provides compositions comprising a lipid carrier and mitochondria as well as methods of delivering exogenous mitochondria to a cell and methods of treating or reversing progression of a disorder associated with mitochondrial dysfunction in a mammalian subject in need thereof.

WO 2017/124037 relates to compositions comprising isolated mitochondria or combined mitochondrial agents and methods of treating disorders using such compositions.

US 20080275005 relates to mitochondrially targeted antioxidant compounds. A compound of the invention comprises a lipophilic cation covalently coupled to an antioxidant moiety.

U.S. Pat. No. 9,855,296 discloses a method for enhancing cardiac or cardiovascular function in a human subject in need thereof, said method comprising administering to said subject a pharmaceutical composition comprising isolated and substantially pure mitochondria in an amount sufficient to enhance said cardiac or cardiovascular function, wherein said mitochondria are syngeneic mitochondria or allogeneic mitochondria.

U.S. Pat. No. 9,603,872 provides methods, kits, and compositions for mitochondrial replacement in the treatment of disorders arising from mitochondrial dysfunction. The invention also features methods of diagnosing neuropsychiatric (e.g., bipolar disorder) and neurodegenerative disorders based on mitochondrial structural abnormalities.

US 20180071337 discloses a therapeutic composition comprising human mitochondria isolated from cells and a pharmaceutically acceptable excipient, wherein the mitochondria can be in a carrier that comprises a lipid bilayer, a vesicle, or a liposome, with or without at least one polypeptide or glycoprotein.

US 20010021526 provides cellular and animal models for diseases associated with mitochondrial defects. Cybrid cell lines which have utility as model systems for the study of disorders that are associated with mitochondrial defects are described.

WO 2013/035101 to the present inventors relates to mitochondrial compositions and therapeutic methods of using same, and discloses compositions of partially purified functional mitochondria and methods of using the compositions to treat conditions which benefit from increased mitochondrial function by administering the compositions to a subject in need thereof.

Attempts to induce transfer of mitochondria into host cells or tissues have been reported. Most methods require active transfer of the mitochondria by injection (e.g. McCully et al. Am J Physiol Heart Circ Physiol. 2009, 296(1):H94-H105). Transfer of mitochondria engulfed within a vehicle, such as a liposome, is also known (e.g. Shi et al. Ethnicity and Disease, 2008; 18(S1):43).

It has been shown that mitochondrial transfer may occur spontaneously between cells in-vitro although it was only established that mtDNA was transferred rather than intact whole functional mitochondria (e.g. Plotnikov et al. Exp Cell Res. 2010, 316(15):2447-55; Spees et al. Proc Natl Acad Sci, 2006; 103(5):1283-8). Mitochondrial transfer in-vitro by endocytosis or internalization has been demonstrated as well (Clark et al., Nature, 1982:295:605-607; Katrangi et al., Rejuvenation Research, 2007; 10(4):561-570).

US 20110105359 provides cryopreserved compositions of cells in the form of self-sustaining bodies, as well as cellular and subcellular fractions. On the other hand, an attempt to inject isolated mitochondria during early reperfusion for cardioprotection showed that cardioprotection requires freshly isolated mitochondria, as frozen mitochondria failed to provide cardioprotection and displayed a significantly decreased oxygen consumption compared with freshly isolated mitochondria (McCully et al., ibid).

WO 2016/008937 relates to methods for the intercellular transfer of mitochondria isolated from a population of donor cells into a population of recipient cells. The methods show improved efficacy of transfer of an amount mitochondria.

US 2012/0107285 is directed to mitochondrial enhancement of cells. Certain embodiments include, but are not limited to, methods of modifying stem cells, or methods of administering modified stem cells to at least one biological tissue.

Aging is among the greatest known risk factors for many human diseases. An age-related disease is a disease that is most often seen with increasing frequency with increasing senescence. Essentially, age-related diseases are complications arising from senescence. Age-related diseases are to be distinguished from the aging process itself because all adult animals age, but not all adult animals experience age-related diseases.

A decline in mitochondrial quality and activity has been associated with normal aging and correlated with the development of a wide range of age-related diseases. Mitochondria contribute to specific aspects of the aging process, including cellular senescence, chronic inflammation, and the age-dependent decline in stem cell activity. A wealth of supportive evidence demonstrates that mitochondrial dysfunction occurs with age due to accumulation of mitochondrial DNA mutations. Various mitochondrial DNA point mutations have been shown to significantly increase with age in the human brain, heart, skeletal muscles and liver tissues. Increased frequency of mitochondrial DNA deletions/insertions have also been reported with increasing age in both animal models and humans. It has been postulated that the replication cycle and the accumulation of mitochondrial DNA mutations might be a conserved mechanism underlying stem cell aging such that mitochondria influence or regulate a number of key aspects of aging (Sun et al., Cell, 2016, 61: 654-66; Srivastava, Genes, 2017, 8:398; Ren et al., Genes, 2017, 8:397).

Cancer is caused by uncontrolled proliferation of abnormal cells in an organ or tissue of the body. Various types of cancer treatments are available, including: surgery, chemotherapy, radiotherapy, immunotherapy, targeted therapy, hormone therapy or stem cell transplant. The cancer treatments often cause severe adverse effects, including: fatigue, nausea and vomiting, anemia, diarrhea, appetite loss, thrombocytopenia, delirium, hair loss, fertility issues, peripheral neuropathy, pain, lymphedema. These debilitating effects diminish the cancer patient's quality of life significantly. The use of bone marrow cells to replenish the bone marrow of cancer patients suffering from hematopoietic malignancies that have undergone bone marrow ablation is well known. Bone marrow transplantation most often uses matched healthy donors. However, in some instances such as multiple myeloma autologous bone marrow can be performed. The use of bone marrow cells to treat non-hematopoietic cancers is not routine in the treatment of those patients.

There is an unmet need to enhance the quality of life of subjects afflicted with debilitating effects due to various conditions, such as aging and age-related diseases as well as cancer patients undergoing chemotherapy or radiation therapy. Reversing the decline in mitochondrial function can slow the effects of aging and diminish age-related diseases as well as debilitating effects of anti-cancer treatment.

### SUMMARY OF THE INVENTION

The present invention provides mammalian stem cells enriched with healthy functional mitochondria and methods for diminishing the debilitating effects of many conditions, including, aging and age-related diseases as well as adverse events of anti-cancer treatments. Unexpectedly, it has now been shown for the first time that transplanting invigorating cells enriched with healthy mitochondria can significantly retard symptoms of aging and advancement of age-related diseases. Furthermore, mitochondrial augmentation therapy using stem cells enriched with healthy mitochondria can alleviate debilitating effects of chemotherapy, radiation therapy and/or immunotherapy with monoclonal antibodies in cancer patients undergoing anti-cancer treatments. In particular, the present invention provides compositions comprising stem cells including autologous or donor stem cells, which have been enriched with functional mitochondria. These cells are useful for alleviating or decreasing the effects of debilitating conditions when introduced into the subject to be treated.

In specific embodiments the subject is treated with stem cells which have been enriched with functional mitochondria obtained from healthy donors. A convenient source for healthy donor mitochondria includes but is not limited to placental mitochondria or mitochondria derived from blood cells. The present invention thus provides methods for the use of allogeneic, autologous or syngeneic “mitochondrially-enriched” stem cells for treating or diminishing the debilitating effects of aging and age-related diseases as well as anti-cancer treatments in cancer patients.

The present invention is based in part on the finding that aging C57BL mice that receive bone marrow cells enriched with healthy mitochondria from murine term placentae show improvement in functional, cognitive and physiological blood tests compared to age matched mice that receive bone marrow not enriched with mitochondria.

According to various embodiments, the source of stem cells may be autologous, syngeneic or from a donor. The provision of stem cells of a subject having a debilitating condition enriched with healthy mitochondria ex-vivo and returned to the same subject provides benefits over other methods involving allogeneic cell therapy. For example, the provided methods eliminate the need to screen the population and find a donor which is human leukocyte antigen (HLA)-matched with the subject, which is a lengthy and costly process, and not always successful. The methods further advantageously eliminate the need for life-long immunosuppression therapy of the subject, so that his body does not reject allogeneic cell populations. Thus, the present invention advantageously provides a unique methodology of ex-vivo therapy, in which human stem cells are removed from the subject's body, enriched ex-vivo with healthy functional mitochondria, and returned to the same subject. Moreover, the present invention relates to the administration of stem cells which, without being bound to any theory or mechanism, are circulating throughout the body in different tissues, to enhance the energy level of the subject and thereby enhance the quality of life for subjects having debilitating conditions.

The present invention is based, in part, on the surprising findings that functional mitochondria can enter intact fibroblasts, hematopoietic stem cells and bone marrow cells, and that treatment of fibroblasts, hematopoietic stem cells and bone marrow cells with functional mitochondria increases mitochondrial content, cell survival and ATP production.

The present invention provides, for the first time, stem cells of aging subjects or cancer patients having augmented or enhanced mitochondrial activity. These stem cells are enriched with healthy functional mitochondria from a suitable source. Typically, the mitochondria may be obtained from blood cells, placental cells, placental cell cultures or other suitable cell lines. Each possibility is a separate embodiment of the invention.

The present invention provides, in one aspect, a method for treating or diminishing debilitating effects of various conditions, by introducing isolated or partially purified frozen-thawed functional human mitochondria into stem cells obtained or derived from a subject afflicted with a debilitating condition or from a donor, and transplanting at least 10⁵ to 2×10⁷ “mitochondrially-enriched” human stem cells per kilogram bodyweight of the patient in a pharmaceutically acceptable liquid medium capable of supporting the viability of the cells into the subject afflicted with the debilitating condition.

According to another aspect, the present invention provides method for treating or diminishing debilitating conditions in a subject comprising administering parenterally a pharmaceutical composition comprising at least 5*10⁵ to 5*10⁹ human stem cells enriched with frozen-thawed healthy functional exogenous mitochondria to the subject, wherein the debilitating conditions are selected from the group consisting of aging, age-related diseases and the sequel of anti-cancer treatments.

According to yet another aspect, the present invention provides a pharmaceutical composition for use in treating or diminishing debilitating conditions in a subject, the pharmaceutical composition comprising at least 10⁵ to 2×10⁷ human stem cells per kilogram bodyweight of the subject, the human stem cells suspended in a pharmaceutically acceptable liquid medium capable of supporting the viability of the cells, wherein the human stem cells are enriched with frozen-thawed healthy functional exogenous mitochondria and wherein the debilitating conditions are selected from the group consisting of aging, age-related diseases and the sequellae of anti-cancer treatments. According to some embodiments, the mitochondrial enrichment of the stem cells comprise introducing into the stem cells a dose of mitochondria of at least 0.088 up to 176 milliunits of CS activity per million cells. According to further embodiments, the mitochondrial enrichment of the stem cells comprise introducing into the stem cells a dose of mitochondria of 0.88 up to 17.6 milliunits of CS activity per million cells.

In some embodiments, the volume of isolated mitochondria is added to the recipient cells at the desired concentration. The ratio of the number of mitochondria donor cells versus the number of mitochondria recipient cells is a ratio above 2:1 (donor cells vs. recipients cells). In typical embodiments, the ratio is at least 5, alternatively at least 10 or higher. In specific embodiments, the ratio of donor cells from which mitochondria are collected to recipient cells is at least 20, 50, 100 or higher. Each possibility is a separate embodiment.

In some embodiments, the subject having the debilitating condition is an aging subject. In certain embodiments, the subject having the debilitating condition suffers from an age-related disease or diseases. In other embodiments, the subject having the debilitating condition is a cancer patient undergoing chemotherapy, radiation therapy, immunotherapy with monoclonal antibodies or a combination thereof. Each possibility represents a separate embodiment of the invention.

In certain embodiments, the healthy functional human exogenous mitochondria are allogeneic mitochondria. In other embodiments, the healthy functional human exogenous mitochondria are autologous or syngeneic, i.e., of the same maternal bloodline.

In another aspect, the present invention provides an ex-vivo method for enriching human stem cells with healthy mitochondria, the method comprising the steps of (i) providing a first composition, comprising a plurality of human stem cells obtained or derived from an individual afflicted with a debilitating condition or from a healthy donor not afflicted with a debilitating condition; (ii) providing a second composition, comprising a plurality of isolated or partially purified frozen-thawed human functional healthy exogenous mitochondria obtained from a healthy donor not afflicted with a debilitating condition; (iii) contacting the human stem cells of the first composition with the frozen-thawed human functional mitochondria of the second composition at a ratio of 0.088-176 mU CS activity per 10⁶ stem cells; and (iv) incubating the composition of (iii) under conditions allowing the frozen-thawed human functional mitochondria to enter the human stem cells thereby enriching said frozen-thawed human stem cells with said human functional mitochondria; wherein the functional mitochondrial content of the enriched human stem cells is detectably higher than the healthy functional mitochondrial content of the human stem cells in the first composition.

In specific embodiments the subject afflicted with a debilitating condition is a cancer patient after treatment with debilitating anti-cancer treatments. Accordingly, the present invention provides an ex-vivo method for enriching human stem cells with healthy functional exogenous mitochondria, the method comprising the steps of (i) providing a first composition, comprising a plurality of human stem cells from an individual afflicted with a malignant disease or from a healthy subject not afflicted with a malignant disease; (ii) providing a second composition, comprising a plurality of isolated or partially purified frozen-thawed human functional mitochondria obtained from the same individual afflicted with the malignant disease prior to anti-cancer treatments or from a healthy subject not afflicted with a malignant disease; (iii) contacting the human stem cells of the first composition with the frozen-thawed human functional mitochondria of the second composition at a ratio of 0.088-176 mU CS activity per 10⁶ stem cells; and (iv) incubating the composition of (iii) under conditions allowing the human functional mitochondria to enter the frozen-thawed human stem cells thereby enriching said human stem cells with said human functional mitochondria; wherein the functional mitochondrial content of the enriched human stem cells is detectably higher than the functional mitochondrial content of the human stem cells in the first composition.

In some embodiments, the conditions allowing the healthy functional human exogenous mitochondria to enter the human stem cells comprise incubating the human stem cells with said healthy functional exogenous mitochondria for a time ranging from 0.5 to 30 hours, at a temperature ranging from 16 to 37° C. In some embodiments, the conditions allowing the healthy functional human exogenous mitochondria to enter the human stem cells comprise incubating the human stem cells with said healthy functional exogenous mitochondria for a time ranging from 0.5 to 30 hours, at a temperature ranging from 16 to 37° C., in a culture medium under an environment supporting cell survival. According to some embodiments the culture medium is saline containing human serum albumin. In some embodiments the conditions for incubation include an atmosphere containing 5% CO₂. In some embodiments the conditions for incubation do not include added CO₂ above the level found in air. Each possibility represents a separate embodiment of the invention.

In some embodiments, the method further comprises centrifugation of the human stem cells and the healthy functional exogenous mitochondria before, during or after incubation. In some embodiments, prior to incubation the method further comprises a single centrifugation of the human stem cells and the healthy functional exogenous mitochondria at a centrifugation force above 2500×g. Each possibility represents a separate embodiment of the invention.

In some embodiments, the mitochondria that have undergone a freeze-thaw cycle demonstrate a comparable oxygen consumption rate following thawing, as compared to control mitochondria that have not undergone a freeze-thaw cycle.

In certain embodiments, the method described above further comprises freezing, and optionally further comprising thawing, the mitochondrially-enriched human stem cells.

In additional embodiments, the human stem cells are expanded before or after mitochondrial augmentation.

The detectable enrichment of the stem cells with functional mitochondria may be determined by functional and/or enzymatic assays, including but not limited to rate of oxygen (O₂) consumption, activity level of citrate synthase, rate of adenosine triphosphate (ATP) production, mitochondrial protein content (such as Succinate dehydrogenase complex, subunit A—SDHA and cytochrome C oxidase—COX1), mitochondrial DNA content. In the alternative the enrichment of the stem cells with healthy donor mitochondria may be confirmed by the detection of mitochondrial DNA (mtDNA) of the donor. According to some embodiments, the extent of enrichment of the stem cells with functional mitochondria may be determined by the level of change in heteroplasmy and/or by the copy number of mtDNA per cell. According to certain exemplary embodiments, the enrichment of the stem cells with healthy functional mitochondria may be determined by conventional assays that are recognized in the art. For example the presence of donor mitochondria can be determined by a method selected from (i) activity level of citrate synthase; or (ii) mtDNA sequencing indicating more than one source of mtDNA. Each possibility represents a separate embodiment of the invention According to some embodiments, the mitochondria may be matched between the donor and the treated subject according to mtDNA haplogroup. According to other embodiments, the mitochondria are chosen according to specific different mtDNA haplogroups prior to stem cell enrichment.

In certain embodiments, the mitochondrial content of the stem cells in the first composition or in the fourth composition is determined by determining the activity level of citrate synthase. Each possibility represents a separate embodiment of the invention.

In certain embodiments, the process of enriching the human stem cells with mitochondria is performed prior to freezing of the cells. In other embodiments, the process of enriching the human stem cells with mitochondria is performed after freezing and thawing of the cells.

In certain embodiments, the autologous human stem cells are frozen and stored prior to affliction with the debilitating condition. In other embodiments, the process of enriching the human stem cells with mitochondria is performed after freezing and thawing of the cells.

In certain embodiments, the stem cells are pluripotent stem cells (PSC). In other embodiments, the PSCs are non-embryonic stem cells. In some embodiments, the stem cells are induced PSCs (iPSCs). In certain embodiments, the stem cells are derived from bone-marrow cells. In particular embodiments the stem cells express the bone marrow hematopoietic progenitor cell antigen CD34 (CD34⁺). In particular embodiments the stem cells are mesenchymal stem cells. In other embodiments, the stem cells are derived from adipose tissue. In yet other embodiments, the stem cells are derived from blood. In further embodiments, the stem cells are derived from umbilical cord blood. In further embodiments the stem cells are derived from oral mucosa. In further embodiments the stem cells comprise common myeloid progenitor cells, common lymphoid progenitor cells or any combination thereof. Each possibility represents a separate embodiment of the invention.

In certain embodiments, the stem cells are bone marrow cells.

In certain embodiments, the stem cells are bone marrow derived stem cells comprising myelopoietic cells. In certain embodiments, the bone marrow derived stem cells comprise erythropoietic cells. In certain embodiments, the bone marrow derived stem cells comprise multi-potential hematopoietic stem cells (HSCs). In certain embodiments, the bone marrow derived stem cells comprise common myeloid progenitor cells, common lymphoid progenitor cells, or any combination thereof. In certain embodiments, the bone marrow derived stem cells comprise megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, or any combination thereof. In certain embodiments, the bone marrow derived stem cells comprise mesenchymal stem cells. Each possibility represents a separate embodiment of the invention.

In particular embodiments, the stem cells are CD34⁺ cells. In certain embodiments, CD34⁺ expressing cells are obtained from umbilical cord blood (i.e., non-bone marrow hematopoietic stem cells). In some embodiments the cells used are autologous stem cells and they may be frozen and stored prior to the debilitating condition related to aging or cancer therapy. In some embodiments the process of enriching the cells with mitochondria is performed prior to freezing. In alternative embodiments the process of enriching the cells with mitochondria is performed after freezing and thawing of the stem cells.

In certain embodiments, the stem cells in the first composition are obtained from an aging subject or from a donor. In certain embodiments, the stem cells in the first composition are bone marrow cells obtained from the bone marrow of an aging subject or from a donor. In certain embodiments, the stem cells in the first composition are directly or indirectly obtained from the bone marrow of the aging subject or from the bone marrow of a donor. In certain embodiments, the stem cells in the first composition are mobilized from the bone marrow of the aging subject or are mobilized from the bone marrow of a donor. In certain embodiments, the stem cells in the first composition are obtained from the peripheral blood of the aging subject or are obtained from the peripheral blood of a donor. Each possibility represents a separate embodiment of the invention.

In certain embodiments, the stem cells in the first composition are obtained from a subject afflicted with a malignant disease. In certain embodiments, the stem cells in the first composition are obtained from a subject afflicted with a non-hematopoietic malignant disease, or from a healthy subject not afflicted with a malignant disease. In certain embodiments, the stem cells in the first composition are obtained from the bone marrow of a subject afflicted with a non-hematopoietic malignant disease, or from a healthy subject not afflicted with a malignant disease. In certain embodiments, the stem cells in the first composition are mobilized from the bone marrow of the subject afflicted with a non-hematopoietic malignant disease, or are mobilized from the bone marrow of a healthy subject not afflicted with a malignant disease. In certain embodiments, the stem cells in the first composition are directly obtained from the bone marrow of the subject afflicted with a non-hematopoietic malignant disease, or are directly obtained from the bone marrow of a healthy subject not afflicted with a malignant disease. In certain embodiments, the stem cells in the first composition are indirectly obtained from the bone marrow of the subject afflicted with a non-hematopoietic malignant disease, or are indirectly obtained from the bone marrow of a healthy subject not afflicted with a malignant disease. In certain embodiments, the bone-marrow cells in the first composition are obtained from the peripheral blood of the subject afflicted with a non-hematopoietic malignant disease, or are obtained from the peripheral blood of a healthy subject not afflicted with a malignant disease. Each possibility represents a separate embodiment of the invention.

In certain embodiments, the stem cells are at least partially purified.

In certain embodiments, the healthy functional mitochondria are derived from a cell or a tissue selected from the group consisting of: placenta, placental cells grown in culture and blood cells.

In certain embodiments, the pharmaceutical composition is administered to the subject suffering from a debilitating condition selected from the group consisting of aging, age-related diseases and the sequellae of anti-cancer treatments. In further embodiments, the pharmaceutical composition is administered to a specific tissue or organ. In yet further embodiments, the pharmaceutical composition is administered by systemic parenteral administration. In other embodiments, the pharmaceutical composition comprising at least about 10⁶ mitochondrially-enriched human stem cells per kilogram body weight of the patient. In additional embodiments, the pharmaceutical composition comprising a total of about 5×10⁵ to 5×10⁹ human stem cells enriched with human mitochondria. In certain embodiments, the administration of the pharmaceutical composition to a subject is by a parenteral route selected from the group consisting of intravenous, intraarterial, intramuscular, subcutaneous, intraperitoneal and direct injection into a tissue or an organ. Each possibility represents a separate embodiment of the invention.

In certain embodiments, the method described above further comprises a preceding step, the step comprising administering to the subject afflicted with the debilitating condition, either aging or a non-hematopoietic malignant disease, or to a healthy donor, an agent who induces mobilization of stem cells from the bone marrow to peripheral blood. In certain embodiments, the agent is selected from the group consisting of granulocyte-colony stimulating factor (G-CSF), granulocyte-macrophage colony-stimulating factor (GM-CSF), 1,1′-[1,4-Phenylenebis(methylene)]-bis[1,4,8,11-tetraazacyclotetradecane] (Plerixafor), a salt thereof, and any combination thereof. Each possibility represents a separate embodiment of the invention. In certain embodiments, the method described above further comprises a step of isolating the stem cells from the peripheral blood of the subject afflicted with the debilitating condition, either aging or a non-hematopoietic malignant disease, or from the peripheral blood of a healthy subject. In certain embodiments, the isolation is performed by apheresis.

In certain embodiments, the method described above further comprise a step of administering to the subject suffering from debilitating conditions selected from the group consisting of aging, age-related diseases and the sequellae of anti-cancer treatments, an agent which prevents, delays, minimizes or abolishes an adverse immunogenic reaction between the subject and the stem cells of the allogeneic donor. In additional embodiments, the functional mitochondria in the second composition are obtained from a subject afflicted with a malignant disease prior to anti-cancer treatments.

In certain embodiments, the method described above further comprises concentrating the stem cells and the functional mitochondria in the third composition before or during incubation. In certain embodiments, the method described above further comprises centrifugation of the third composition before, during or after incubation. Each possibility represents a separate embodiment of the invention.

In alternative embodiments, the aging subject or subject that suffers from an age-related disease or diseases is transplanted with stem cells enriched with mitochondria. In certain embodiments, the stem cells are from a donor not afflicted with an age-related disease. In specific embodiments the stem cells are autologous bone marrow stem cells. In certain embodiments, the stem cells in the first composition are mobilized from the bone marrow of the aging subject or subject afflicted with age-related disease or diseases, or are mobilized from the bone marrow of a healthy donor not afflicted with age-related diseases. In certain embodiments, the stem cells in the first composition are obtained from the peripheral blood of the aging subject or subject afflicted with age-related disease or diseases, or are obtained from the peripheral blood of a healthy donor not afflicted with age-related diseases. Each possibility represents a separate embodiment of the invention.

In alternative embodiments the subject suffers from a hematopoietic malignancy and the stem cells transplanted into the subject are enriched with mitochondria. In certain embodiments, the stem cells are from a healthy donor not afflicted with a malignant disease. In specific embodiments the stem cells are autologous bone marrow stem cells for example such as are used in various hematopoietic malignancies including multiple myeloma and certain types of lymphoma. According to these embodiments, the stem cells in the first composition are obtained from the bone marrow of the subject afflicted with a hematopoietic malignant disease, or are obtained from the bone marrow of a healthy subject not afflicted with a malignant disease. In certain embodiments, the stem cells in the first composition are mobilized from the bone marrow of the subject afflicted with a hematopoietic malignant disease, or are mobilized from the bone marrow of a healthy subject not afflicted with a malignant disease. In certain embodiments, the stem cells in the first composition are obtained from the peripheral blood of the subject afflicted with a hematopoietic malignant disease, or are obtained from the peripheral blood of a healthy subject not afflicted with a malignant disease. Each possibility represents a separate embodiment of the invention.

In certain embodiments, the method described above further comprises a preceding step, the step comprising administering to a subject an agent which induces mobilization of bone marrow stem cells from the bone marrow to peripheral blood. In certain embodiments, the agent is selected from the group consisting of granulocyte-colony stimulating factor (G-CSF), granulocyte-macrophage colony-stimulating factor (GM-CSF), 1,1′-[1,4-Phenylenebis(methylene)]-bis[1,4,8,11-tetraazacyclotetradecane] (Plerixafor), a salt thereof, and any combination thereof. Each possibility represents a separate embodiment of the invention. In certain embodiments, the method described above further comprises a step of isolating the stem cells from the peripheral blood of the subject afflicted with a hematopoietic malignant disease or from the peripheral blood of a healthy subject not afflicted with a malignant disease. In certain embodiments, the isolation is performed by apheresis.

In certain embodiments, the method described above further comprises concentrating the stem cells and the functional mitochondria in composition (iii) before or during incubation. In certain embodiments, the method described above further comprises centrifugation of composition (iii) before, during or after incubation. Each possibility represents a separate embodiment of the invention.

In certain embodiments, the stem cells in the first composition are obtained from a subject having a debilitating condition selected from aging, age-related diseases and a malignant disease undergoing a debilitating therapy, and have (i) a decreased rate of oxygen (O₂) consumption; (ii) a decreased activity level of citrate synthase; (iii) a decreased rate of adenosine triphosphate (ATP) production; or (iv) any combination of (i), (ii) and (iii), as compared to a subject not afflicted with the debilitating condition. Each possibility represents a separate embodiment of the invention.

In certain embodiments, the stem cells in the first composition are obtained from a healthy donor not afflicted with a debilitating condition, having (i) a normal rate of oxygen (O₂) consumption; (ii) a normal activity level of citrate synthase; (iii) a normal rate of adenosine triphosphate (ATP) production; or (iv) any combination of (i), (ii) and (iii). Each possibility represents a separate embodiment of the invention. In certain embodiments, the isolated or partially purified human functional mitochondria in the second composition are obtained from a donor not afflicted with a debilitating condition, having normal mitochondrial DNA. As used herein the term “normal mitochondrial DNA” refers to mitochondrial DNA not having any deletion or mutation that is known to be associated with a primary mitochondrial disease.

In certain embodiments, the stem cells enriched with healthy functional mitochondria have (i) an increased rate of oxygen (O₂) consumption; (ii) an increased activity level of citrate synthase; (iii) an increased rate of adenosine triphosphate (ATP) production; (iv) an increased normal mitochondrial DNA content; or (v) any combination of (i), (ii), (iii) and (iv), as compared to the stem cells prior to mitochondrial enrichment. Each possibility represents a separate embodiment of the invention.

According to certain exemplary embodiments, the stem cells enriched with healthy functional mitochondria have (i) an increased activity level of citrate synthase; and (ii) an increased normal mitochondrial DNA content; as compared to the stem cells prior to mitochondrial enrichment.

In certain embodiments, the total amount of mitochondrial proteins in the partially purified mitochondria is between 20%-80% of the total amount of cellular proteins within the sample. Exemplary methods for obtaining such compositions of isolated or partially purified mitochondria are disclosed in WO 2013/035101.

The present invention further provides, in another aspect, a plurality of human stem cells enriched with healthy mitochondria, obtained by any one of the embodiments of the methods described above. Explicitly, it is to be understood that the human stem cells enriched with functional mitochondria according to the present invention are not derived from a subject afflicted with a primary mitochondrial disease. According to some specific embodiments the stem cells enriched with healthy mitochondria are other than bone marrow stem cells.

The present invention further provides, in another aspect, a plurality of human stem cells enriched ex-vivo with mitochondria, wherein the stem cells have at least one property selected from the group consisting of (a) an increased mitochondrial DNA content; (b) an increased activity level of citrate synthase; (c) an increased content of at least one mitochondrial protein selected from SDHA and COX1; (d) an increased rate of oxygen (O₂) consumption; (e) an increased rate of ATP production; or (f) any combination thereof, relative to the corresponding level in the stem cells prior to mitochondrial enrichment. Each possibility represents a separate embodiment of the invention.

According to some embodiments the stem cells are CD34⁺ stem cells. The human stem cells enriched ex-vivo with functional mitochondria according to the present invention are not derived from a subject afflicted with a primary mitochondrial disease.

In certain embodiments, the total amount of mitochondrial proteins in the partially purified mitochondria is between 20%-80% of the total amount of cellular proteins within the sample.

In certain embodiments, the plurality of human stem cells described above are CD34⁺ and have an increased mitochondrial content; an increased mitochondrial DNA content; an increased rate of oxygen (O₂) consumption; an increased activity level of citrate synthase, as compared to the stem cells prior to mitochondrial enrichment. In some embodiments the increased content or activity is higher than the content or activity than that in the cells at the time of isolation.

The present invention further provides, in another aspect, a pharmaceutical composition comprising a plurality of the human bone marrow stem cells enriched ex-vivo with healthy functional mitochondria as described above.

The present invention further provides, in another aspect, the pharmaceutical composition described above for use in treating a human subject afflicted with a debilitating condition. According to certain embodiments, the subject afflicted with a debilitating condition is an aging subject. In certain embodiments, the subject afflicted with a debilitating condition suffers from age-related disease or diseases. In some embodiments, the subject afflicted with a debilitating condition suffers from a malignant disease undergoing a debilitating therapy. In further embodiments the pharmaceutical composition described above is used for treating a human subject in remission or after recovery from a malignant disease.

The present invention further provides, in another aspect, a method of treating a human subject afflicted with a debilitating condition, comprising the step of administering to the patient the pharmaceutical composition described above. According to certain embodiments, the subject afflicted with a debilitating condition is an aging subject. In certain embodiments, the subject afflicted with a debilitating condition suffers from age-related disease or diseases. In some embodiments, the subject afflicted with a debilitating condition suffers from a malignant disease undergoing a debilitating therapy. In further embodiments the pharmaceutical composition described above is used for treating a human subject in remission or after recovery from a malignant disease. In certain embodiments, the stem cells comprising the pharmaceutical composition are autologous or syngeneic to the subject afflicted with the debilitating condition. In certain embodiments, the stem cells comprising the pharmaceutical composition are allogeneic to the subject afflicted with the debilitating condition. Each possibility represents a separate embodiment of the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is three micrographs showing mouse fibroblast cell expressing mitochondrial GFP (left panel), incubation with isolated RFP-labeled mitochondria (middle panel), and an overlay (right panel), obtained by fluorescence confocal microscopy.

FIG. 2 is a bar graph showing a comparison of ATP levels in mouse fibroblast cells which were either untreated (Control), treated with a mitochondrial complex I irreversible inhibitor (Rotenone), or treated with Rotenone and mouse placental mitochondria (Rotenone+Mitochondria). Data is presented as mean values±SEM, (*) p value<0.05. RLU—relative luminescence units.

FIG. 3 is four micrographs obtained by fluorescence confocal microscopy showing mouse bone-marrow cells incubated with GFP-labeled mitochondria isolated from mouse melanoma cells.

FIG. 4 is a bar graph illustrating the level of C57BL mtDNA in the bone marrow of FVB/N mice at various time points after IV injection of bone marrow cells enriched with exogenous mitochondria from C57BL mouse.

FIG. 5 is a bar graph showing a comparison of citrate synthase (CS) activity in mouse bone marrow (BM) cells incubated with varying amounts of GFP-labeled mitochondria isolated from mouse melanoma cells, with or without centrifugation.

FIG. 6A is a bar graph showing a comparison of CS activity in murine BM cells after enrichment with increasing amounts of GFP-labeled mitochondria. FIG. 6B is a bar graph showing a comparison of cytochrome c reductase activity in these cells (black bars), compared to the activity in GFP-labeled mitochondria (gray bar).

FIG. 7A is a bar graph illustrating the number of copies of C57BL mtDNA in FVB/N bone marrow cells after incubation of the cells with exogenous mitochondria from C57BL mouse in various concentrations (0.044, 0.44, 0.88, 2.2, 4.4, 8.8, 17.6 mUnits CS activity), compared to untreated cells (NT). FIG. 7B is a bar graph illustrating the content of mtDNA encoded (COX1) protein in FVB/N bone marrow cells after incubation of the cells with exogenous mitochondria from C57BL mouse in various concentrations (0.044, 0.44, 0.88, 2.2, 4.4, 8.8, 17.6 mUnits CS activity), compared to untreated cells (NT), normalized to Janus levels. FIG. 7C is a bar graph illustrating the content of nuclear encoded (SDHA) protein in FVB/N bone marrow cells after incubation of the cells with exogenous mitochondria from C57BL mouse in various concentrations (0.044, 0.44, 0.88, 2.2, 4.4, 8.8, 17.6 mUnits CS activity), compared to untreated cells (NT), normalized to Janus levels.

FIG. 8A is a bar graph showing a comparison of CS activity in control, untreated human BM cells and human BM cells incubated with GFP-labeled mitochondria isolated from human placental cells, with or without centrifugation. FIG. 8B is a bar graph showing a comparison of ATP levels in control, untreated human BM cells and human BM cells incubated with GFP-labeled mitochondria isolated from human placental cells, with centrifugation.

FIG. 9A depict the result of a FACS analysis in human BM cells not incubated with GFP-labeled mitochondria. FIG. 9B depict the result of a FACS analysis in human BM cells incubated with GFP-labeled mitochondria after centrifugation.

FIG. 10A is a bar graph showing ATP content of human CD34⁺ cells from a healthy donor not treated (NT) or treated with blood derived mitochondria (MNV-BLD). FIG. 10B is a bar graph showing CS activity of human CD34⁺ cells from a healthy donor treated or not treated with blood derived mitochondria.

FIG. 11 is three micrographs obtained by fluorescence confocal microscopy CD34+ cells incubated with GFP-labeled mitochondria isolated from HeLa-TurboGFP-Mitochondria cells.

FIG. 12A is an illustration of mtDNA deletion in Pearson-patient cord blood cells as well as a southern blot analysis showing the deletion. FIG. 12B is a bar graph illustrating the number of human mtDNA copies in the bone marrow of NSGS mice 2 month after mitochondrial augmentation therapy using Pearson's cord blood cells enriched with human mitochondria (UCB+Mito), as compared to mice injected with non-augmented cord blood cells (UCB).

FIG. 13A is a bar graph showing FVB/N ATP8 mutated mtDNA levels in the bone marrow of FVB/N mice 1 month post administration of stem cells enriched with healthy functional mitochondria obtained from C57BL placenta. FIG. 13B is a bar graph showing FVB/N ATP8 mutated mtDNA levels in the livers of FVB/N mice 3 months post administration of stem cells enriched with healthy functional mitochondria obtained from C57BL placenta.

FIG. 14A-14C is graph bars illustrating the biodistribution of bone marrow cells enriched with mitochondria by the amount of C57BL mtDNA in the bone marrow (FIG. 14A), brain (FIG. 14B) and heart (FIG. 14C) of mice up to 3 months after MAT. White bars and associated dots indicate augmented bone marrow samples, grey bars are controls.

FIG. 15 is a bar graph showing a comparison of FVB/N ATP8 mutated mtDNA levels in the brains of FVB/N mice 1 month post administration of stem cells enriched with healthy functional wild type mitochondria (isolated from liver of C57BL mice), in untreated FVB/N mice (Naive), FVB/N mice administered with stem cells enriched with C57BL healthy liver mitochondria (C57BL Mito), FVB/N mice administered with stem cells enriched with C57BL healthy mitochondria and were subjected to total body irradiation (TBI) prior to stem cells administration (TBI C57BL Mito) and FVB/N mice administered with stem cells enriched with C57BL healthy mitochondria and were subjected to Busulfan chemotherapeutic agent prior to stem cell administration (Busulfan C57BL Mito).

FIGS. 16A-16C show line graphs illustrating open field behavioral test performance of 12-month old C57BL/6J mice treated with: mitochondria-enriched BM cells (MNV-BM-PLC, 1×10⁶ cells), bone marrow cells (BM control, 1×10⁶ cells) or a control vehicle solution (control, 4.5% Albumin in 0.9% w/v NaCl), before treatment and 9 months post treatment.

FIG. 16A shows quantification of the distance moved during the open field test.

FIG. 16B shows center duration (time (s) or % change from baseline); FIG. 16C shows wall duration (time (s) or % change from baseline).

FIG. 16D is a line graph illustrating blood urea nitrogen (BUN) levels in 12 months old C57BL/6J mice treated with: mitochondria-enriched BM cells (MNV-BM-PLC, 1×10⁶ cells), bone marrow cells (BM control, 1×10⁶ cells) or a control vehicle solution (control, 4.5% Albumin in 0.9% w/v NaCl), before treatment and 9 months post treatment.

FIGS. 16E-16F show bar graphs illustrating Rotarod test of 12-month old C57BL/6J mice administered treated with either mitochondria-enhanced bone marrow (BM) cells (MNV-BM-PLC, 1×10⁶ cells), bone marrow cells (BM, 1×10⁶ cells) or a control vehicle solution (VEHICLE, 4.5% Albumin in 0.9% w/v NaCl). The results presented are before treatment and 1 and 3 months after treatment. FIG. 16E shows Rotarod score (in seconds (s)), of the various treated test groups at the indicated time points. FIG. 16F shows Rotarod score (presented as percentage from baseline, of the various treated test groups at the indicated time points.

FIGS. 16G-16J show bar graph illustrating strength test of 12-month old C57BL/6J mice administered treated with either mitochondria-enhanced bone marrow (BM) cells (MNV-BM-PLC, 1×10⁶ cells), bone marrow cells (BM, 1×10⁶ cells) or a control vehicle solution (VEHICLE, 4.5% Albumin in 0.9% w/v NaCl). The results presented are before treatment and 1 and 3 months after treatment. FIGS. 16G-16H—grip strength (force) (g or % change from baseline); FIGS. 16I-16J—grip strength time (time (s) or % change from baseline).

FIG. 17A is a scheme depicting the course of treatment and evaluation in the clinical trial performed on patient 1, a young Pearson Syndrome (PS) and PS-related Fanconi Syndrome (FS) patient, with a deletion mutation in his mtDNA, encompassing ATP8. FIG. 17B is a bar graph showing aerobic Metabolic Equivalent of Task (MET) score pre administration of stem cells enriched with functional mitochondria, 2.5 months and 8 months post administration of the enriched stem cells. FIG. 17C is a bar graph illustrating the level of lactate in the blood of a PS patient treated by the methods provided in the present invention as a function of time before and after therapy. FIG. 17D is a line graph illustrating the standard deviation score of the weight and height of a PS patient treated by the methods provided in the present invention as a function of time before and after therapy. FIG. 17E is a line graph illustrating the alkaline phosphatase (ALP) level of a PS patient treated by the methods provided in the present invention as a function of time before and after therapy. FIG. 17F is a line graph illustrating the long term elevation in blood red blood cell (RBC) levels in a PS patient before and after therapy provided by the present invention. FIG. 17G is a line graph illustrating the long term elevation in blood hemoglobin (HGB) levels in a PS patient before and after therapy provided by the present invention. FIG. 17H is a line graph illustrating the long term elevation in blood hematocrit (HCT) levels in a PS patient before and after therapy provided by the present invention. FIG. 17I is a line graph illustrating the creatinine level of a PS patient treated by the methods provided in the present invention as a function of time before and after therapy. FIG. 17J is a line graph illustrating the bicarbonate level of a PS patient treated by the methods provided in the present invention as a function of time before and after therapy. FIG. 17K is a line graph illustrating the level of base excess of a PS patient treated by the methods provided in the present invention as a function of time before and after therapy. FIG. 17L is a bar graph illustrating the levels of blood magnesium in a PS patient treated by the methods provided in the present invention as a function of time before and after therapy, before and after magnesium supplementation. FIG. 17M is a bar graph illustrating the glucose to creatinine ratio in the urine of a PS patient treated by the methods provided in the present invention as a function of time before and after therapy. FIG. 17N is a bar graph illustrating the potassium to creatinine ratio in the urine of a PS patient treated by the methods provided in the present invention as a function of time before and after therapy. FIG. 17O is a bar graph illustrating the chloride to creatinine ratio in the urine of a PS patient treated by the methods provided in the present invention as a function of time before and after therapy. FIG. 17P is a bar graph illustrating the sodium to creatinine ratio in the urine of a PS patient treated by the methods provided in the present invention as a function of time before and after therapy.

FIG. 18A is a line graph illustrating the normal mtDNA content in 3 PS patients (Pt.1, Pt.2 and Pt.3) treated by the methods provided in the present invention as a function of time before and after therapy, as measured by digital PCR for the deleted region (in each patient) compared to the 18S genomic DNA representing number of normal mtDNA per cell, and normalized per baseline.

FIG. 18B is a line graph illustrating the heteroplasmy level (deleted mtDNA compared to total mtDNA) in 3 PS patients (Pt.1, Pt.2 and Pt.3), at baseline after MAT. Dotted line represents the baseline for each patient.

FIG. 19A is another scheme of the different stages of treatment of a Pearson Syndrome (PS) patient, as further provided by the present invention. FIG. 19B is a bar graph illustrating the level of lactate in the blood of a PS patient treated by the methods provided in the present invention as a function of time before (B) and after therapy. FIG. 19C is a bar graph illustrating the sit-to-stand score of a PS patient treated by the methods provided in the present invention as a function of time before and after therapy. FIG. 19D is a bar graph illustrating the six-minute-walk-test score of a PS patient treated by the methods provided in the present invention as a function of time before and after therapy. FIG. 19E is a bar graph illustrating the dynamometer score of three consecutive repetitions (R1, R2, R3) of a PS patient treated by the methods provided in the present invention as a function of time before and after therapy. FIG. 19F is a bar graph illustrating the urine magnesium to creatinine ratio in a PS patient treated by the methods provided in the present invention as a function of time before and after therapy. FIG. 19G is a bar graph illustrating the urine potassium to creatinine ratio in a PS patient treated by the methods provided in the present invention as a function of time before and after therapy. FIG. 19H is a bar graph illustrating the urine calcium to creatinine ratio in a PS patient treated by the methods provided in the present invention as a function of time before and after therapy. FIG. 19I is a bar graph illustrating the ATP8 to 18S copy number ratio in the urine of a PS patient treated by the methods provided in the present invention as a function of time before and after therapy. FIG. 19J is a bar graph illustrating the ATP level in lymphocytes of a PS patient treated by the methods provided in the present invention as a function of time before and after therapy.

FIG. 20A is yet another scheme of the different stages of treatment of a Pearson Syndrome (PS) patient and of a Kearns-Sayre syndrome (KSS) patient, as further provided by the present invention. FIG. 20B is a bar graph illustrating the level of lactate in the blood of a PS patient treated by the methods provided in the present invention as a function of time before (B) and after therapy. FIG. 20C is a bar graph illustrating the AST and ALT levels of a PS patient treated by the methods provided in the present invention as a function of time before and after therapy. FIG. 20D is a bar graph illustrating the triglyceride, total cholesterol and VLDL cholesterol levels of a PS patient treated by the methods provided in the present invention as a function of time before and after therapy. FIG. 20E is a bar graph illustrating the hemoglobin A1C (HbAlC) score of a PS patient treated by the methods provided in the present invention as a function of time before and after therapy. FIG. 20F is a line graph illustrating the sit-to-stand score of a PS patient (Pt.3) treated by the methods provided in the present invention as a function of time before and after therapy. FIG. 20G is a line graph illustrating the six-minute-walk-test score of a PS patient (Pt.3) treated by the methods provided in the present invention as a function of time before and after therapy.

FIG. 21 is a bar graph illustrating the ATP content in the peripheral blood of a KSS patient treated by the methods provided in the present invention, before and after therapy.

### DETAILED DESCRIPTION OF THE INVENTION

The present invention provides cellular platforms, more specifically stem cell-derived cellular platforms, for targeted and systemic delivery of therapeutically-significant amounts of fully functional, healthy mitochondria and methods for their utilization in subjects having a debilitating condition, comprising aging subjects and subjects suffering from age-related disease or diseases, as well as cancer patients suffering from the sequellae of anti-cancer treatments including chemotherapy, radiation therapy or immunotherapy with monoclonal antibodies. The present invention is based on several surprising findings, amongst which are clinical results exemplified herein, showing that intravenous injection of bone marrow-derived hematopoietic stem cells enriched with normal, functional, healthy mitochondria can beneficially affect various tissues of the subject. In other words, improvement in function can be achieved in various organs and tissues following the administration of stem cells enriched with healthy mitochondria.

The present invention is based in part on the finding that bone marrow cells are receptive to being enriched with intact functional mitochondria and that human bone marrow cells are particularly receptive to being enriched with mitochondria as disclosed for example in WO 2016/135723. Without being bound to any theory or mechanism, it is postulated that co-incubation of stem cells with healthy mitochondria promotes the transition of intact functional mitochondria into the stem cells.

It has also been found that the extent of enrichment of stem cells, including but not limited to bone marrow-derived hematopoietic stem cells, with mitochondria and improvement in the cells' mitochondrial functionality are dependent on conditions used for mitochondrial enrichment, including but not limited to the concentration of the isolated or partially purified mitochondria, as well as the incubation conditions, and thus may be manipulated, in order to produce the desired enrichment.

The present invention provides, in one aspect, a method for treating and/or diminishing debilitating effects of various conditions, by introducing ex vivo partially purified healthy human mitochondria into stem cells obtained or derived from a subject afflicted with a debilitating condition or from a healthy donor, and transplanting the “mitochondrially-enriched” stem cells into the subject afflicted with the debilitating condition.

In certain embodiments, the subject afflicted with the debilitating condition suffers from aging or an age-related disease or diseases. In other embodiments, the subject afflicted with the debilitating effects is a cancer patient undergoing chemotherapy, radiation therapy or immunotherapy with monoclonal antibodies. In some embodiments, the cancer patient is a subject afflicted with a non-hematopoietic malignant disease. In other embodiments, the cancer patient is a subject afflicted with a hematopoietic malignant disease.

In further embodiments, the human stem cells administered to the subject are autologous to the subject. In other embodiments, the human stem cells administered to the subject are from a donor, i.e., allogeneic to the subject.

In some embodiments, the autologous or allogeneic human stem cells are pluripotent stem cells (PSCs) or induced pluripotent stem cells (iPSCs). In further embodiments, the autologous or allogeneic human stem cells are mesenchymal stem cells.

According to several embodiments, the human stem cells are derived from adipose tissue, oral mucosa, blood, umbilical cord blood or bone marrow. Each possibility represents a separate embodiment of the present invention. In specific embodiments, the human stem cells are derived from bone marrow.

In another aspect, the current invention provides a pharmaceutical composition for use in treating or diminishing debilitating conditions in a subject, the pharmaceutical composition comprising at least 10⁵ to 2×10⁷ human stem cells per kilogram bodyweight of the subject, the human stem cells suspended in a pharmaceutically acceptable liquid medium capable of supporting the viability of the cells, wherein the human stem cells are enriched with frozen-thawed healthy functional exogenous mitochondria and wherein the debilitating conditions are selected from the group consisting of aging, age-related diseases and the sequellae of anti-cancer treatments

In some embodiments, the pharmaceutical composition comprises at least 10⁵ to 2×10⁷ mitochondrially-enriched human stem cells per kilogram bodyweight of the patient. In some embodiments, the pharmaceutical composition comprises at least 5×10⁵ to 1.5×10⁷ mitochondrially-enriched human stem cells per kilogram bodyweight of the patient. In some embodiments, the pharmaceutical composition comprises at least 5×10⁵ to 4×10⁷ mitochondrially-enriched human stem cells per kilogram bodyweight of the patient. In some embodiments, the pharmaceutical composition comprises at least 10⁶ to 10⁷ mitochondrially-enriched human stem cells per kilogram bodyweight of the patient. In other embodiments, the pharmaceutical composition comprises at least 10⁵ or at least 10⁶ mitochondrially-enriched human stem cells per kilogram bodyweight of the patient. Each possibility represents a separate embodiment of the present invention. In some embodiments, the pharmaceutical composition comprises a total of at least 5×10⁵ up to 5×10⁹ mitochondrially-enriched human stem cells. In some embodiments, the pharmaceutical composition comprises a total of at least 10⁶ up to 10⁹ mitochondrially-enriched human stem cells. In other embodiments, the pharmaceutical composition comprises a total of at least 2×10⁶ up to 5×10⁸ mitochondrially-enriched human stem cells.

In another aspect, the present invention provides an ex-vivo method for enriching human stem cells with functional mitochondria, the method comprising the steps of (i) providing a first composition, comprising a plurality of human stem cells obtained or derived from a subject afflicted with a debilitating condition or from a healthy donor not afflicted with a debilitating condition; (ii) providing a second composition, comprising a plurality of isolated or partially purified human functional mitochondria obtained from a healthy donor not afflicted with a debilitating condition; (iii) contacting the human stem cells of the first composition with the human functional mitochondria of the second composition, thus forming a third composition; and (iv) incubating the third composition under conditions allowing the human functional mitochondria to enter the human stem cells thereby enriching said human stem cells with said human functional mitochondria, thus forming a fourth composition; wherein the mitochondrial content of the enriched human stem cells in the fourth composition is detectably higher than the mitochondrial content of the human stem cells in the first composition.

The present invention provides, in one aspect, an ex-vivo method for enriching human bone-marrow cells with functional mitochondria, the method comprising the steps of (i) providing a first composition, comprising a plurality of human bone-marrow cells obtained or derived from a patient afflicted with a malignant disease or from a healthy subject not afflicted with a malignant disease; (ii) providing a second composition, comprising a plurality of isolated human functional mitochondria obtained from the same patient afflicted with the malignant disease prior to anti-cancer treatments or from a healthy subject not afflicted with a malignant disease; (iii) mixing the human bone-marrow cells of the first composition with the human functional mitochondria of the second composition, thus forming a third composition; and (iv) incubating the third composition under conditions allowing the human functional mitochondria to enter the human bone-marrow cells thereby enriching said human bone-marrow cells with said human functional mitochondria, thus forming a fourth composition; wherein the mitochondrial content of the human bone-marrow cells in the fourth composition is detectably higher than the mitochondrial content of the human bone-marrow cells in the first composition.

The term “ex-vivo method” as used herein refers to a method comprising steps performed exclusively outside the human body. In particular, an ex vivo method comprises manipulation of cells outside the body that are subsequently reintroduced or transplanted into the subject to be treated.

The term “enriching” as used herein refers to any action designed to increase the mitochondrial content, e.g. the number of intact mitochondria, or the functionality of mitochondria of a mammalian cell. In particular, stem cells enriched with functional mitochondria will show enhanced function compared to the same stem cells prior to enrichment.

The term “stem cells” as used herein generally refers to any mammalian stem cells. Stem cells are undifferentiated cells that can differentiate into other types of cells and can divide to produce more of the same type of stem cells. Stem cells can be either totipotent or pluripotent.

The term “human stem cells” as used herein generally refers to all stem cells naturally found in humans, and to all stem cells produced or derived ex vivo and are compatible with humans. A “progenitor cell”, like a stem cell, has a tendency to differentiate into a specific type of cell, but is already more specific than a stem cell and is pushed to differentiate into its “target” cell. The most important difference between stem cells and progenitor cells is that stem cells can replicate indefinitely, whereas progenitor cells can divide only a limited number of times. The term “human stem cells” as used herein further includes “progenitor cells” and “non-fully differentiated stem cells”.

In some embodiments, enrichment of the stem cells with healthy functional human exogenous mitochondria comprises washing the mitochondrially-enriched stem cells after incubation of the human stem cells with said healthy functional human exogenous mitochondria. This step provides a composition of the mitochondrially-enriched stem cells substantially devoid of cell debris or mitochondrial membrane remnants and mitochondria that did not enter the stem cells. In some embodiments, washing comprises centrifugation of the mitochondrially-enriched stem cells after incubation of the human stem cells with said healthy functional human exogenous mitochondria. According to some embodiments, the pharmaceutical composition comprising the mitochondrially-enriched human stem cells is separated from free mitochondria, i.e., mitochondria that did not enter the stem cells, or other cell debris. According to some embodiments, the pharmaceutical composition comprising the mitochondrially-enriched human stem cells does not comprise a detectable amount of free mitochondria.

As used herein the term “pluripotent stem cells (PSCs)” refers to cells that can propagate indefinitely, as well as give rise to a plurality of cell types in the body. Totipotent stem cells are cells that can give rise to every other cell type in the body. Embryonic stem cells (ESCs) are totipotent stem cells and induced pluripotent stem cells (iPSCs) are pluripotent stem cells.

As used herein the term “induced pluripotent stem cells (iPSCs)” refers to a type of pluripotent stem cell that can be generated from human adult somatic cells.

As used herein the term “embryonic stem cells (ESC)” refers to a type of totipotent stem cell derived from the inner cell mass of a blastocyst.

The term “bone marrow cells” as used herein generally refers to all human cells naturally found in the bone marrow of humans, and to all cell populations naturally found in the bone marrow of humans. The term “bone marrow stem cells” and “bone marrow-derived stem cells” refer to the stem cell population derived from the bone marrow.

The terms “functional mitochondria” and “healthy mitochondria” are used herein interchangeably and refer to mitochondria displaying parameters indicative of normal mtDNA and normal, non-pathological levels of activity. The activity of mitochondria can be measured by a variety of methods well known in the art, such as membrane potential, O₂ consumption, ATP production, and citrate synthase (CS) activity level.

The phrase “stem cells obtained from a subject afflicted with a debilitating condition or from a donor not afflicted with a debilitating condition” as used herein refers to cells that were stem cells in the subject/donor at the time of their isolation from the subject.

The phrase “stem cells derived from a subject afflicted with a debilitating condition or from a donor not afflicted with a debilitating condition” as used herein refers to cells that were not stem cells in the subject/donor, and have been manipulated to become stem cells. The term “manipulated” as used herein refers to the use of any one of the methods known in the field (Yu J. et al, Science, 2007, Vol. 318(5858), pages 1917-1920) for reprograming somatic cells to an undifferentiated state and becoming induced pluripotent stem cells (iPSCs), and, optionally, further reprograming the iPSCs to become cells of a desired lineage or population (Chen M. et al., IOVS, 2010, Vol. 51(11), pages 5970-5978), such as bone marrow cells (Xu Y. et al., PLoS ONE, 2012, Vol. 7(4), page e34321).

The term “CD34⁺ cells” as used herein refers to hematopoietic stem cells characterized as being CD34 positive that are obtained from stem cells or mobilized from bone marrow or obtained from umbilical cord blood.

The term “a subject afflicted with debilitating condition” as used herein refers to a human subject experiencing debilitating effects caused by certain conditions. The debilitating condition may refer to aging, age-related diseases or cancer patient undergoing anti-cancer treatments, as well as other debilitating conditions.

The term “aging” refers to an inevitable progressive deterioration of physiological function with increasing age, demographically characterized by an age-dependent increase in mortality and decline of various physical and mental abilities.

The term “age-related disease” as used herein refers to “diseases of the elderly”, diseases seen with increasing frequency with increasing senescence. Age-related diseases include, but are not limited to atherosclerosis and cardiovascular disease, cancer, arthritis, cataracts, osteoporosis, type 2 diabetes, hypertension and dementia such as Alzheimer's disease. The incidence of all of these diseases increases cumulatively with advancing age.

The term “a subject afflicted with a malignant disease” as used herein refers to a human subject diagnosed with a malignant disease, suspected to have a malignant disease, or in a risk group of developing a malignant disease. As certain types of malignancies are inherited, the progeny of subjects diagnosed with a malignant disease are considered a risk group of developing a malignant disease.

The term “a subject/donor not afflicted with a malignant disease” as used herein refers to human subject not diagnosed with a malignant disease, and/or not suspected to have a malignant disease.

The term “a subject afflicted with a non-hematopoietic malignant disease” as used herein refers to human subject diagnosed with a non-hematopoietic malignant disease, and/or suspected to have a non-hematopoietic malignant disease.

The term “a subject afflicted with a hematopoietic malignant disease” as used herein refers to human subject diagnosed with a hematopoietic malignant disease, and/or suspected to have a hematopoietic malignant disease.

The term “healthy donor” and “healthy subject” are used interchangeably, and refer to a subject not suffering from the disease or condition which is being treated.

The term “contacting” refers to bringing the composition of mitochondria and cells into sufficient proximity to promote entry of the mitochondria into the cells. The term introducing mitochondria into the target cells is used interchangeably with the term contacting.

The term “isolated or partially purified human functional mitochondria” as used herein refers to intact mitochondria isolated from cells obtained from a healthy subject, not afflicted with a mitochondrial disease. The total amount of mitochondrial proteins in the partially purified mitochondria is between 20%-80% of the total amount of cellular proteins within the sample.

The term “isolated” as used herein and in the claims in the context of mitochondria includes mitochondria that were purified, at least partially, from other components found in said source. In certain embodiments, the total amount of mitochondrial proteins in the second composition comprising the plurality of isolated healthy functional exogenous mitochondria, is between 20%-80%, 20-70%, 40-70%, 20-40%, or 20-30% of the total amount of cellular proteins within the sample. Each possibility represents a separate embodiment of the present invention. In certain embodiments, the total amount of mitochondrial proteins in the second composition comprising the plurality of isolated healthy functional exogenous mitochondria, is between 20%-80% of the total amount of cellular proteins within the sample. In certain embodiments, the total amount of mitochondrial proteins in the second composition comprising the plurality of isolated healthy functional exogenous mitochondria, is between 20%-80% of the combined weight of the mitochondria and other sub-cellular fractions. In other embodiments, the total amount of mitochondrial proteins in the second composition comprising the plurality of isolated healthy functional exogenous mitochondria, is above 80% of the combined weight of the mitochondria and other sub-cellular fractions.

According to some embodiments, the method for enriching human stem cells with healthy functional exogenous mitochondria does not comprise measuring the membrane potential of the cell.

In some embodiments, the enrichment of the stem cells with healthy functional exogenous mitochondria comprises introducing into the stem cells a dose of mitochondria of at least 0.044 up to 176 milliunits of CS activity per million cells. In some embodiments, the enrichment of the stem cells with healthy functional exogenous mitochondria comprises introducing into the stem cells a dose of mitochondria of at least 0.088 up to 176 milliunits of CS activity per million cells. In other embodiments, the enrichment of the stem cells with healthy functional exogenous mitochondria comprises introducing into the stem cells a dose of mitochondria of at least 0.2 up to 150 milliunits of CS activity per million cells. In other embodiments, the enrichment of the stem cells with healthy functional exogenous mitochondria comprises introducing into the stem cells a dose of mitochondria of at least 0.4 up to 100 milliunits of CS activity per million cells. In some embodiments, the enrichment of the stem cells with healthy functional exogenous mitochondria comprises introducing into the stem cells a dose of mitochondria of at least 0.6 up to 80 milliunits of CS activity per million cells. In some embodiments, the enrichment of the stem cells with healthy functional exogenous mitochondria comprises introducing into the stem cells a dose of mitochondria of at least 0.7 up to 50 milliunits of CS activity per million cells. In some embodiments, the enrichment of the stem cells with healthy functional exogenous mitochondria comprises introducing into the stem cells a dose of mitochondria of at least 0.8 up to 20 milliunits of CS activity per million cells. In some embodiments, the enrichment of the stem cells with healthy functional exogenous mitochondria comprises introducing into the stem cells a dose of mitochondria of at least 0.88 up to 17.6 milliunits of CS activity per million cells. In some embodiments, the enrichment of the stem cells with healthy functional exogenous mitochondria comprises introducing into the stem cells a dose of mitochondria of at least 0.44 up to 17.6 milliunits of CS activity per million cells.

Mitochondrial dose can be expressed in terms of units of CS activity or mtDNA copy number of other quantifiable measurements of the amount of healthy functional mitochondria as explained herein. A “unit of CS activity” is defined as the amount that enables conversion of one micromole substrate in 1 minute in 1 mL reaction volume.

In some embodiments, the identification/discrimination of endogenous mitochondria from exogenous mitochondria, after the latter have been introduced into the target cell, can be performed by various means, including, for example, but not limited to: identifying differences in mtDNA sequences, for example different haplotypes, between the endogenous mitochondria and exogenous mitochondria, identifying specific mitochondrial proteins originating from of the source tissue of the exogenous mitochondria, such as, for example, cytochrome p450 cholesterol side chain cleavage (P450SCC) from placenta, UCP1 from brown adipose tissue, and the like, or any combination thereof.

The term “exogenous” with regard to mitochondria refers to mitochondria that are introduced to a target cell (for example, stem cells), from a source which is external to the cell. For example, in some embodiments, exogenous mitochondria are commonly derived or isolated from a donor cell which is different than the target cell. For example, exogenous mitochondria may be produced/made in a donor cell, purified/isolated obtained from the donor cell and thereafter introduced into the target cell.

The term “endogenous” with regard to mitochondria refers to mitochondria that is being made/expressed/produced by cell and is not introduced from an external source into the cell. In some embodiments, endogenous mitochondria contain proteins and/or other molecules which are encoded by the genome of the cell. In some embodiments, the term “endogenous mitochondria” is equivalent to the term “host mitochondria”.

As used herein, the term “autologous cells” or “cells that are autologous, refers to being the patient's own cells. The term “autologous mitochondria”, refers to mitochondria obtained from the patient's own cells or from maternally related cells. The terms “allogeneic cells” or “allogeneic mitochondria”, refer to being from a different donor individual.

The term “syngeneic” as used herein and in the claims refers to genetic identity or genetic near-identity sufficient to allow grafting among individuals without rejection. The term syngeneic in the context of mitochondria is used herein interchangeably with the term autologous mitochondria meaning of the same maternal bloodline

The term “exogenous mitochondria” refers to a mitochondria or mitochondrial DNA that are introduced to a target cell (i.e., stem cell), from a source which is external to the cell. For example, in some embodiments, an exogenous mitochondria may be derived or isolated from a cell which is different than the target cell. For example, an exogenous mitochondria may be produced/made in a donor cell, purified/isolated obtained from the donor cell and thereafter introduced into the target cell.

The phrase “conditions allowing the human functional mitochondria to enter the human stem cells” as used herein generally refers to parameters such as time, temperature, culture medium and proximity between the mitochondria and the stem cells. For example, human cells and human cell lines are routinely incubated in liquid medium, and kept in sterile environments, such as in tissue culture incubators, at 37° C. and 5% CO₂ atmosphere. According to alternative embodiments disclosed and exemplified herein the cells may be incubated at room temperature in saline supplemented with human serum albumin According to some embodiments, the incubation of the human functional mitochondria with the human stem cells is preceded by centrifugation. According to other embodiments, the incubation occurs prior to centrifugation. In yet further embodiments, the centrifugation occurs during said incubation. In certain embodiments, the centrifugation speed is 8,000 g. In certain embodiments, the centrifugation speed is 7,000 g. According to further embodiments, the centrifugation is at a speed between 5,000-10,000 g. According to further embodiments, the centrifugation is at a speed between 7,000-8,000 g.

In certain embodiments, the human stem cells are incubated with the healthy functional exogenous mitochondria for a time ranging from 0.5 to 30 hours, at a temperature ranging from about 16 to about 37° C. In certain embodiments, the human stem cells are incubated with the healthy functional exogenous mitochondria for a time ranging from 1 to 30 or from 5 to 25 hours. Each possibility represents a separate embodiment of the present invention. In specific embodiments, incubation is for 20 to 30 hours. In some embodiments, incubation is for at least 1, 5, 10, 15 or 20 hours. Each possibility represents a separate embodiment of the present invention. In other embodiments, incubation is up to 5, 10, 15, 20 or 30 hours. Each possibility represents a separate embodiment of the present invention. In specific embodiments, incubation is for 24 hours. In some embodiments, incubation is at room temperature (16° C. to 30° C.). In other embodiments, incubation is at 37° C. In some embodiments, incubation is in a 5% CO₂ atmosphere. In other embodiments, incubation does not include added CO₂ above the level found in air. In certain embodiments, incubation is until the mitochondrial content in the stem cells is increased in average by 1% to 45% compared to their initial mitochondrial content.

In yet further embodiments, the incubation is performed in culture medium supplemented with human serum albumin (HSA). In additional embodiments, the incubation is performed in saline supplemented with HSA. According to certain exemplary embodiments, the conditions allowing the functional mitochondria to enter the human stem cells thereby enriching said human stem cells with said human functional mitochondria include incubation at room temperature in saline supplemented with 4.5% human serum albumin.

By manipulating the conditions of the incubation, one can manipulate the features of the product. In certain embodiments, the incubation is performed at 37° C. In certain embodiments, the incubation is performed for at least 6 hours. In certain embodiments, the incubation is performed for at least 12 hours. In certain embodiments, the incubation is performed for 12 to 24 hours. In certain embodiments, the incubation is performed at a ratio of 1*10⁵ to 1*10⁷ naïve stem cells per amount of exogenous mitochondria having or exhibiting 4.4 units of CS. In certain embodiments, the incubation is performed at a ratio of 1*10⁶ naïve stem cells per amount of exogenous mitochondria having or exhibiting 4.4 units of CS. In certain embodiments, the conditions are sufficient to increase the mitochondrial content of the naïve stem cells by at least about 3%, 5% or 10% as determined by CS activity. Each possibility represents a separate embodiment of the present invention.

The term “mitochondrial content” as used herein refers to the amount of functional mitochondria within a cell, or to the average amount of functional mitochondria within a plurality of cells.

As used herein and in the claims, the term “mitochondrial disease” and the term “primary mitochondrial disease” may be used interchangeably. The term “primary mitochondrial disease” as used herein refers to a mitochondrial disease which is diagnosed by a known or indisputably pathogenic mutation in the mitochondrial DNA, or by mutations in genes of the nuclear DNA, whose gene products are imported into the mitochondria. According to some embodiments, the primary mitochondrial disease is a congenital disease. According to some embodiments, the primary mitochondrial disease is not a secondary mitochondrial dysfunction. The terms “secondary mitochondrial dysfunction” and “acquired mitochondrial dysfunction” are used interchangeably throughout the application.

In certain embodiments, the methods described above in various embodiments thereof, further include centrifugation before, during or after incubation of the stem cells with the exogenous mitochondria. Each possibility represents a separate embodiment of the present invention. In some embodiments, the methods described above in various embodiments thereof include a single centrifugation step before, during or after incubation of the stem cells with the exogenous mitochondria. In some embodiments, the centrifugation force ranges from 1000 g to 8500 g. In some embodiments, the centrifugation force ranges from 2000 g to 4000 g. In some embodiments, the centrifugation force is above 2500 g. In some embodiments, the centrifugation force ranges from 2500 g to 8500 g. In some embodiments, the centrifugation force ranges from 2500 g to 8000 g. In some embodiments, the centrifugation force ranges from 3000 g to 8000 g. In other embodiments, the centrifugation force ranges from 4000 g to 8000 g. In specific embodiments, the centrifugation force is 7000 g. In other embodiments, the centrifugation force is 8000 g. In some embodiments, centrifugation is performed for a time ranging from 2 minutes to 30 minutes. In some embodiments, centrifugation is performed for a time ranging from 3 minutes to 25 minutes. In some embodiments, centrifugation is performed for a time ranging from 5 minutes to 20 minutes. In some embodiments, centrifugation is performed for a time ranging from 8 minutes to 15 minutes.

In some embodiments, centrifugation is performed in a temperature ranging from 4 to 37° C. In certain embodiments, centrifugation is performed in a temperature ranging from 4 to 10° C. or 16-30° C. Each possibility represents a separate embodiment of the present invention. In specific embodiments, centrifugation is performed at 2-6° C. In specific embodiments, centrifugation is performed at 4° C. In some embodiments, the methods described above in various embodiments thereof include a single centrifugation before, during or after incubation of the stem cells with the exogenous mitochondria, followed by resting the cells at a temperature lower than 30° C. In some embodiments, the conditions allowing the human functional mitochondria to enter the human stem cells include a single centrifugation before, during or after incubation of the stem cells with the exogenous mitochondria, followed by resting the cells at a temperature ranging between 16 to 28° C.

In certain embodiments, the first composition is fresh. In certain embodiments, the first composition was frozen and then thawed prior to incubation. In certain embodiments, the second composition is fresh. In certain embodiments, the second composition was frozen and then thawed prior to incubation. In certain embodiments, the fourth composition is fresh. In certain embodiments, the fourth composition was frozen and then thawed prior to administration.

In specific embodiments, the stem cells obtained from a patient afflicted with a malignant disease or from a healthy subject are bone marrow cells or bone marrow-derived stem cells.

The term “mammalian stem cells enriched with functional mitochondria” refers to human and non-human mammals.

According to the principles of the present invention, healthy functional human exogenous mitochondria are introduced into human stem cells, thus enriching these cells with healthy functional human mitochondria. It should be understood that such enrichment changes the mitochondrial content of the human stem cells: while naïve human stem cells substantially have one population of host/autologous mitochondria, human stem cells enriched with exogenous mitochondria substantially have two populations of mitochondria, a first population of host/autologous/endogenous mitochondria and another population of the introduced mitochondria (i.e., the exogenous mitochondria). Thus, the term “enriched” relates to the state of the cells after receiving/incorporation exogenous mitochondria. Determining the number and/or ratio between the two populations of mitochondria is straightforward, as the two populations may differ in several aspects e.g. in their mitochondrial DNA. Therefore, the phrase “human stem cells enriched with healthy functional human mitochondria” is equivalent to the phrase “human stem cells comprising endogenous mitochondria and healthy functional exogenous mitochondria”. For example, human stem cells which comprise at least 1% healthy functional exogenous mitochondria of the total mitochondria, are considered comprising host/autologous/endogenous mitochondria and healthy functional exogenous mitochondria in a ratio of 99:1. For example, “3% of the total mitochondria” means that after enrichment the original (endogenous) mitochondrial content is 97% of the total mitochondria and the introduced (exogenous) mitochondria is 3% of the total mitochondria—this is equivalent to (3/97=) 3.1% enrichment. Another example—“33% of the total mitochondria” means that after enrichment, the original (endogenous) mitochondrial content is 67% of the total mitochondria and the introduced (exogenous) mitochondria is 33% of the total mitochondria—this is equivalent to (33/67=) 49.2% enrichment.

Heteroplasmy is the presence of more than one type of mitochondrial DNA within a cell or individual. The heteroplasmy level is the proportion of mutant mtDNA molecules vs. wild type/functional mtDNA molecules and is an important factor in considering the severity of mitochondrial diseases. While lower levels of heteroplasmy (sufficient amount of mitochondria are functional) are associated with a healthy phenotype, higher levels of heteroplasmy (insufficient amount of mitochondria are functional) are associated with pathologies. In certain embodiments, the heteroplasmy level of the stem cells in the fourth composition is at least 1% lower than the heteroplasmy level of the stem cells in the first composition. In certain embodiments, the heteroplasmy level of the stem cells in the fourth composition is at least 3% lower than the heteroplasmy level of the stem cells in the first composition. In certain embodiments, the heteroplasmy level of the stem cells in the fourth composition is at least 5% lower than the heteroplasmy level of the stem cells in the first composition. In certain embodiments, the heteroplasmy level of the stem cells in the fourth composition is at least 10% lower than the heteroplasmy level of the stem cells in the first composition. In certain embodiments, the heteroplasmy level of the stem cells in the fourth composition is at least 15% lower than the heteroplasmy level of the stem cells in the first composition. In certain embodiments, the heteroplasmy level of the stem cells in the fourth composition is at least 20% lower than the heteroplasmy level of the stem cells in the first composition. In certain embodiments, the heteroplasmy level of the stem cells in the fourth composition is at least 25% lower than the heteroplasmy level of the stem cells in the first composition. In certain embodiments, the heteroplasmy level of the stem cells in the fourth composition is at least 30% lower than the heteroplasmy level of the stem cells in the first composition.

In certain embodiments, the mitochondrial content of the human stem cells enriched with healthy mitochondria (also referred to herein as cells of the fourth composition) is detectably higher than the mitochondrial content of the human stem cells in the first composition. According to various embodiments the mitochondrial content of the fourth composition is at least 5%, at least 10%, at least 25%, at least 50%, at least 100%, at least 200% or more, higher than the mitochondrial content of the first composition. In certain embodiments, the first composition is used fresh.

In certain embodiments, the first composition is frozen and then stored and used after thawing. In other embodiments, the second composition comprising a plurality of functional human mitochondria is used fresh. In further embodiments, the second composition is frozen and thawed prior to use. In further embodiments the fourth composition is used without freezing and storage. In yet further embodiments the fourth composition is used after freezing, storage and thawing. Methods suitable for freezing and thawing of cell preparations in order to preserve viability are well known in the art. Methods suitable for freezing and thawing of mitochondrial in order to preserve the structure and function are disclosed in WO 2013/035101 and WO 2016/135723 to the present inventors and references cited therein.

Citrate synthase (CS) is localized in the mitochondrial matrix, but is encoded by nuclear DNA. Citrate synthase is involved in the first step of the Krebs cycle, and is commonly used as a quantitative enzyme marker for the presence of intact mitochondria (Larsen S. et al., J. Physiol., 2012, Vol. 590(14), pages 3349-3360; Cook G. A. et al., Biochim. Biophys. Acta., 1983, Vol. 763(4), pages 356-367).

In certain embodiments, the mitochondrial content of the stem cells in the first composition, in the second composition or in the fourth composition is determined by determining the content of citrate synthase. In certain embodiments, the mitochondrial content of the stem cells in the first composition, in the second composition or in the fourth composition is determined by determining the activity level of citrate synthase. In certain embodiments, the mitochondrial content of the stem cells in the first composition, in the second composition or in the fourth composition correlates with the content of citrate synthase. In certain embodiments, the mitochondrial content of the stem cells in the first composition, in the second composition or in the fourth composition correlates with the activity level of citrate synthase. CS activity can be measured by commercially available kits e.g., using the CS activity kit CS0720 (Sigma).

Eukaryotic NADPH-cytochrome C reductase (cytochrome C reductase) is a flavoprotein localized to the endoplasmic reticulum. It transfers electrons from NADPH to several oxygenases, the most important of which are the cytochrome P450 family of enzymes, responsible for xenobiotic detoxification. Cytochrome C reductase is widely used as an endoplasmic reticulum marker. In certain embodiments, the second composition is substantially free from cytochrome C reductase or cytochrome C reductase activity. In certain embodiments, the fourth composition is not enriched with cytochrome C reductase or cytochrome C reductase activity compared to the first composition

In certain embodiments, the stem cells are pluripotent stem cells (PSC). In other embodiments, the PSCs are non-embryonic stem cells. According to some embodiments embryonic stem cells are explicitly excluded from the scope of the invention. In some embodiments, the stem cells are induced PSCs (iPSCs). In certain embodiments, the stem cells are embryonic stem cells. In certain embodiments, the stem cells are derived from bone-marrow cells. In particular embodiments the stem cells are CD34⁺ cells. In particular embodiments the stem cells are mesenchymal stem cells. In other embodiments, the stem cells are derived from adipose tissue. In yet other embodiments, the stem cells are derived from blood. In further embodiments, the stem cells are derived from umbilical cord blood. In further embodiments the stem cells are derived from oral mucosa.

In certain embodiments, the bone-marrow derived stem cells comprise myelopoietic cells. The term “myelopoietic cells” as used herein refers to cells involved in myelopoiesis, e.g. in the production of bone-marrow and of all cells that arise from it, namely, all blood cells.

In certain embodiments, the bone-marrow derived stem cells comprise erythropoietic cells. The term “erythropoietic cells” as used herein refers to cells involved in erythropoiesis, e.g. in the production of red blood cells (erythrocytes).

In certain embodiments, the bone-marrow derived stem cells comprise multi-potential hematopoietic stem cells (HSCs). The term “multi-potential hematopoietic stem cells” or “hemocytoblasts” as used herein refers to the stem cells that give rise to all the other blood cells through the process of hematopoiesis.

In certain embodiments, the bone-marrow derived stem cells comprise common myeloid progenitor cells, common lymphoid progenitor cells, or any combination thereof. In certain embodiments, the bone-marrow derived stem cells comprise mesenchymal stem cells. The term “common myeloid progenitor” as used herein refers to the cells that generate myeloid cells. The term “common lymphoid progenitor” as used herein refers to the cells that generate lymphocytes.

In certain embodiments, the bone-marrow derived stem cells of the first composition further comprise megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, or any combination thereof. Each possibility represents a separate embodiment of the invention.

In certain embodiments, the bone-marrow derived stem cells comprise mesenchymal stem cells. The term “mesenchymal stem cells” as used herein refers to multipotent stromal cells that can differentiate into a variety of cell types, including osteoblasts (bone cells), chondrocytes (cartilage cells), myocytes (muscle cells) and adipocytes (fat cells).

In certain embodiments, the bone-marrow derived stem cells consist of myelopoietic cells. In certain embodiments, the bone-marrow derived stem cells consist of erythropoietic cells. In certain embodiments, the bone-marrow derived stem cells consist of multi-potential hematopoietic stem cells (HSCs). In certain embodiments, the bone-marrow derived stem cells consist of common myeloid progenitor cells, common lymphoid progenitor cells, or any combination thereof. In certain embodiments, the bone-marrow derived stem cells consist of megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, or any combination thereof. In certain embodiments, the bone-marrow derived stem cells consist of mesenchymal stem cells. Each possibility represents a separate embodiment of the invention.

Hematopoietic progenitor cell antigen CD34, also known as CD34 antigen, is a protein that in humans is encoded by the CD34 gene. CD34 is a cluster of differentiation in a cell surface glycoprotein and functions as a cell-cell adhesion factor. In certain embodiments, the bone-marrow stem cells express the bone-marrow progenitor cell antigen CD34 (are CD34⁺). In certain embodiments, the bone marrow stem cells present the bone-marrow progenitor cell antigen CD34 on their external membrane. In certain embodiments the CD34⁺ cells are from umbilical cord blood.

In certain embodiments, the stem cells in the first composition are directly derived from the subject afflicted with a debilitating condition. In certain embodiments, the stem cells in the first composition are directly derived from a donor not afflicted with a debilitating condition. The term “directly derived” as used herein refers to stem cells which were derived directly from other cells. In certain embodiments, the hematopoietic stem cells (HSC) were derived from bone-marrow cells. In certain embodiments, the hematopoietic stem cells (HSC) were derived from peripheral blood.

In certain embodiments, the stem cells in the first composition are indirectly derived from the subject afflicted with a debilitating condition. In certain embodiments, the stem cells in the first composition are indirectly derived from a donor not afflicted with a debilitating condition. The term “indirectly derived” as used herein refers to stem cells which were derived from non-stem cells. In certain embodiments, the stem cells were derived from somatic cells which were manipulated to become induced pluripotent stem cells (iPSCs).

In certain embodiments, the stem cells in the first composition are directly obtained from the bone marrow of the subject afflicted with a debilitating condition. In certain embodiments, the stem cells in the first composition are directly obtained from the bone-marrow of a donor not afflicted with a debilitating condition. The term “directly obtained” as used herein refers to stem cells which were obtained from the bone-marrow itself, e.g. by means such as surgery or suction through a needle by a syringe.

In certain embodiments, the stem cells in the first composition are indirectly obtained from the bone marrow of the patient afflicted with a debilitating condition. In certain embodiments, the stem cells in the first composition are indirectly obtained from the bone marrow of a donor not afflicted with a debilitating condition. The term “indirectly obtained” as used herein refers to bone marrow cells which were obtained from a location other than the bone marrow itself.

In certain embodiments, the stem cells in the first composition are obtained from the peripheral blood of the subject afflicted with a debilitating condition. In certain embodiments, the stem cells in the first composition are obtained from the peripheral blood of the subject not afflicted with a debilitating condition or from the peripheral blood of the subject not afflicted with a debilitating condition. The term “peripheral blood” as used herein refers to blood circulating in the blood system.

In certain embodiments, the first composition comprises a plurality of human bone marrow stem cells obtained from peripheral blood, wherein said first composition further comprises megakaryocytes, erythrocytes, mast cells, myeloblasts, basophils, neutrophils, eosinophils, monocytes, macrophages, natural killer (NK) cells, small lymphocytes, T lymphocytes, B lymphocytes, plasma cells, reticular cells, or any combination thereof. Each possibility represents a separate embodiment of the invention.

In certain embodiments, the method described above further comprises a preceding step, the step comprising administering to the subject afflicted with a debilitating condition an agent which induces mobilization of bone-marrow cells to peripheral blood. In certain embodiments, the method described above further comprises a preceding step, the step comprising administering to a donor not afflicted with a debilitating condition an agent which induces mobilization of bone-marrow cells to peripheral blood.

In certain embodiments, the agent which induces mobilization of bone-marrow cells/stem cells produced in the bone marrow to peripheral blood is selected from the group consisting of granulocyte-colony stimulating factor (G-CSF), granulocyte-macrophage colony-stimulating factor (GM-CSF), 1,1′-[1,4-Phenylenebis(methylene)]bis[1,4,8,11-tetraazacyclotetradecane] (Plerixafor, CAS number 155148-31-5), a salt thereof, and any combination thereof. Each possibility represents a separate embodiment of the invention.

In certain embodiments, the method described above further comprises a step of isolating the stem cells from the peripheral blood of the subject afflicted with a debilitating condition. In certain embodiments, the method described above further comprises a step of isolating the stem cells from the peripheral blood of a donor not afflicted with a debilitating disease. The term “isolating from the peripheral blood” as used herein refers to the isolation of stem cells from other constituents of the blood.

During apheresis, the blood of a subject or donor is passed through an apparatus that separates out one particular constituent and returns the remainder to the circulation. It is thus a medical procedure which is performed outside the body. In certain embodiments, the isolation is performed by apheresis.

In certain embodiments, the method described above further comprises concentrating the stem cells and the functional mitochondria in the third composition before incubation. In certain embodiments, the method described above further comprises concentrating the stem cells and the functional mitochondria in the third composition during incubation.

In certain embodiments, the method described above further comprises centrifugation of the third composition before incubation. In other embodiments, the method described above further comprises centrifugation of the third composition during incubation. In certain embodiments, the method described above further comprises centrifugation of the third composition after incubation.

In certain embodiments, the stem cells in the first composition are obtained from a subject afflicted with a debilitating condition, and the stem cells have (i) a normal rate of oxygen (O₂) consumption; (ii) a normal content or activity level of citrate synthase; (iii) a normal rate of adenosine triphosphate (ATP) production; or (iv) any combination of (i), (ii) and Each possibility represents a separate embodiment of the invention.

In certain embodiments, the stem cells in the first composition are obtained from a subject afflicted with a debilitating condition, and the stem cells have (i) a decreased rate of oxygen (O₂) consumption; (ii) a decreased content or activity level of citrate synthase; (iii) a decreased rate of adenosine triphosphate (ATP) production; or (iv) any combination of (i), (ii) and (iii), as compared to a subject not afflicted with a debilitating condition. Each possibility represents a separate embodiment of the invention.

It should be emphasized that any reference to any measurable feature or characteristic or aspect directed to a plurality of cells or mitochondria is directed to the measurable average feature or characteristic or aspect of the plurality of cells or mitochondria.

In certain embodiments, the stem cells in the first composition are obtained from a donor not afflicted with a debilitating condition, and have (i) a normal rate of oxygen (O₂) consumption; (ii) a normal content or activity level of citrate synthase; (iii) a normal rate of adenosine triphosphate (ATP) production; or (iv) any combination of (i), (ii) and (iii). Each possibility represents a separate embodiment of the invention.

In certain embodiments, the isolated human functional mitochondria in the second composition are obtained from a healthy subject, with normal mitochondrial DNA and have (i) a normal rate of oxygen (O₂) consumption; (ii) a normal content or activity level of citrate synthase; (iii) a normal rate of adenosine triphosphate (ATP) production; or (iv) any combination of (i), (ii) and Each possibility represents a separate embodiment of the invention.

In certain embodiments, the stem cells in the fourth composition have (i) an increased rate of oxygen (O₂) consumption; (ii) an increased content or activity level of citrate synthase; (iii) an increased rate of adenosine triphosphate (ATP) production; (iv) an increased mitochondrial DNA content or (v) any combination of (i), (ii), (iii) and (iv), as compared to the stem cells in the first composition. Each possibility represents a separate embodiment of the invention.

The term “increased rate of oxygen (O₂) consumption” as used herein refers to a rate of oxygen (O₂) consumption which is detectably higher than the rate of oxygen (O₂) consumption in the first composition, prior to mitochondria enrichment.

The term “increased content or activity level of citrate synthase” as used herein refers to a content or activity level of citrate synthase which is detectably higher than the content value or activity level of citrate synthase in the first composition, prior to mitochondria enrichment.

The term “increased rate of adenosine triphosphate (ATP) production” as used herein refers to a rate of adenosine triphosphate (ATP) production which is detectably higher than the rate of adenosine triphosphate (ATP) production in the first composition, prior to mitochondria enrichment.

The term “increased mitochondrial DNA content” as used herein refers to the content of mitochondrial DNA which is detectably higher than the mitochondrial DNA content in the first composition, prior to mitochondria enrichment. Mitochondrial content may be determined by measuring SDHA or COX1 content. “Normal mitochondrial DNA” in the context of the specification and claims refers to mitochondrial DNA not carrying/having a mutation or deletion that is known to be associated with a mitochondrial disease. The term “normal rate of oxygen (O₂) consumption” as used herein refers to the average O₂ consumption of cells from healthy individuals. The term “normal activity level of citrate synthase” as used herein refers to the average activity level of citrate synthase in cells from healthy individuals. The term “normal rate of adenosine triphosphate (ATP) production” as used herein refers to the average ATP production rate in cells from healthy individuals.

According to some aspects, the present invention provides a method of treating debilitating conditions or a symptom thereof in a human patient in need of such treatment, the method comprising the step of administering a pharmaceutical composition comprising a plurality of human stem cells to the patient, wherein the human stem cells are enriched with frozen-thawed healthy functional exogenous mitochondria without a pathogenic mutation in mitochondrial DNA.

In certain embodiments, the symptom is selected from the group consisting of impaired walking capability, impaired motor skills, impaired language skills, impaired memory, weight loss, cachexia, low blood alkaline phosphatase levels, low blood magnesium levels, high blood creatinine levels, low blood bicarbonate levels, low blood base excess levels, high urine glucose/creatinine ratios, high urine chloride/creatinine ratios, high urine sodium/creatinine ratios, high blood lactate levels, high urine magnesium/creatinine ratios, high urine potassium/creatinine ratios, high urine calcium/creatinine ratios, glucosuria, magnesuria, high blood urea levels, low C-Peptide level, high HbAlC level, hypoparathyroidism, ptosis, hearing loss, cardiac conduction disorder, low ATP content and oxygen consumption in lymphocytes, mood disorders including bipolar disorder, obsessive compulsive disorder, depressive disorders, as well as personality disorders. Each possibility represents a separate embodiment of the present invention. It should be understood that defining symptoms as “high” and “low” correspond to “detectably higher than normal” and “detectably lower than normal”, respectively, wherein the normal level is the corresponding level in a plurality of subjects not afflicted with a mitochondrial disease.

In certain embodiments, the pharmaceutical composition is administered to a specific tissue or organ. In certain embodiments, the pharmaceutical composition comprises at least 10⁴ mitochondrially-enriched human stem cells. In certain embodiments, the pharmaceutical composition comprises about 10⁴ to about 10⁸ mitochondrially-enriched human stem cells.

In certain embodiments, the pharmaceutical composition is administered by parenteral administration. In certain embodiments, the pharmaceutical composition is administered by systemic administration. In certain embodiments, the pharmaceutical composition is administered by intravenous injection. In certain embodiments, the pharmaceutical composition is administered by intravenous infusion. In certain embodiments, the pharmaceutical composition comprises at least 10⁵ mitochondrially-enriched human stem cells. In certain embodiments, the pharmaceutical composition comprises about 10⁶ to about 10⁸ mitochondrially-enriched human stem cells. In certain embodiments, the pharmaceutical composition comprises at least about 10⁵-2*10⁷ mitochondrially-enriched human stem cells per kilogram body weight of the patient. In certain embodiments, the pharmaceutical composition comprises at least about 10⁵ mitochondrially-enriched human stem cells per kilogram body weight of the patient. In certain embodiments, the pharmaceutical composition comprises about 10⁵ to about 2*10⁷ mitochondrially-enriched human stem cells per kilogram body weight of the patient. In certain embodiments, the pharmaceutical composition comprises about 10⁶ to about 5*10⁶ mitochondrially-enriched human stem cells per kilogram body weight of the patient.

Mitochondrial DNA content may be measured by performing quantitative PCR of a mitochondrial gene prior and post mitochondrial enrichment, normalized to a nuclear gene.

In specific situations the same cells, prior to mitochondria enrichment, serve as controls to measure CS and ATP activity and determine enrichment level.

In certain embodiments, the term “detectably higher” as used herein refers to a statistically-significant increase between the normal and increased values. In certain embodiments, the term “detectably higher” as used herein refers to a non-pathological increase, i.e. to a level in which no pathological symptom associated with the substantially higher value becomes apparent. In certain embodiments, the term “increased” as used herein refers to a value which is 1.05 fold, 1.1 fold, 1.25 fold, 1.5 fold, 2 fold, 3 fold, 4 fold, 5 fold, 6 fold, 7 fold or higher than the corresponding value found in corresponding cells or corresponding mitochondria of a healthy subject or of a plurality of healthy subjects or in the stem cells of the first composition prior to mitochondrial enrichment. Each possibility represents a separate embodiment of the invention.

In certain embodiments, the stem cells in the fourth composition have at least one of (i) an increased normal mitochondrial DNA content compared to the mitochondrial DNA content in the stem cells prior to mitochondrial enrichment; (ii) an increased rate of oxygen (O₂) consumption compared to the rate of oxygen (O₂) consumption in stem cells prior to mitochondrial enrichment; (iii) an increased content or activity level of citrate synthase compared to the content or activity level of citrate synthase in stem cells prior to mitochondrial enrichment; (iv) an increased rate of adenosine triphosphate (ATP) production compared to the rate of adenosine triphosphate (ATP) production in stem cells prior to mitochondrial enrichment; or (v) any combination of (i), (ii), (iii) and (iv). Each possibility represents a separate embodiment of the invention.

In certain embodiments, the total amount of mitochondrial proteins in the second composition is between 20%-80% of the total amount of cellular proteins within the sample.

As used herein the term “about” refers to ±10% of the indicated numerical value. Typically, the numerical values as used herein refer to ±10% of the indicated numerical value.

In certain embodiments, the method further comprises freezing the fourth composition. In certain embodiments, the method further comprises freezing and then defrosting the fourth composition.

The present invention further provides, in another aspect, a plurality of human stem cells enriched with functional mitochondria, obtained by the method described above.

In certain embodiments, the plurality of stem cells is frozen before enrichment with functional mitochondria. In further embodiments, the plurality of stem cells is frozen and then thawed before enrichment with functional mitochondria. In other embodiments, the plurality of stem cells enriched with functional mitochondria is frozen. In other embodiments, the plurality of stem cells enriched with functional mitochondria is frozen and then thawed before use.

The present invention further provides, in another aspect, a plurality of human stem cells, wherein the stem cells have at least one property selected from the group consisting of (a) an increased mitochondrial content (b) an increased rate of oxygen (O₂) consumption; (c) an increased content or activity level of citrate synthase; (d) increased mitochondrial DNA content or (e) any combination of (a), (b), (c) and (d), compared to human stem cells from the same source prior to enrichment with healthy mitochondria, according to the principles of the invention. Each possibility represents a separate embodiment of the invention. According to some embodiments the stem cells are CD34⁺ stem cells.

The term “increased mitochondrial content” as used herein refers to a mitochondrial content which is detectably higher than the mitochondrial content of the first composition, prior to mitochondria enrichment.

In certain embodiments, the plurality of cells is frozen. In certain embodiments, the plurality of cells is frozen and then thawed before use.

In certain embodiments, the plurality of human stem cells are CD34⁺ and have an increased mitochondrial content; an increased level of normal mitochondrial DNA; an increased rate of oxygen (O₂) consumption; an increased activity level of citrate synthase. Each possibility represents a separate embodiment of the present invention.

In certain embodiments, the plurality of human stem cells have an increased mitochondrial content; an increased level of normal mitochondrial DNA; an increased rate of oxygen (O₂) consumption; and having an increased activity level of citrate synthase.

The present invention further provides, in another aspect, a pharmaceutical composition comprising a plurality of human stem cells enriched with functional mitochondria as described above.

The term “pharmaceutical composition” as used herein refers to any composition comprising cells further comprising a medium or carrier in which the cells are maintained in a viable state.

In certain embodiments, the pharmaceutical composition is frozen. In certain embodiments, the pharmaceutical composition is frozen and then thawed before use.

In certain embodiments, the pharmaceutical composition described above is for use in a method of treating certain symptoms in a human subject having a debilitating condition. The term “treating” as used herein includes the diminishment, alleviation, or amelioration of at least one symptom associated with or induced by the debilitating effects of the condition afflicted on the subject.

The present invention further provides, in another aspect, a method of alleviating or diminishing the debilitating effects conditions, including, but not limited to aging, age-related diseases or anti-cancer therapies in a human subject afflicted with a malignant disease, comprising the step of administering to the subject the pharmaceutical composition described above.

The term “method” as used herein generally refers to manners, means, techniques and procedures for accomplishing a given task, including, but not limited to, those manners, means, techniques and procedures either known to, or readily developed from known manners, means, techniques and procedures by practitioners of the chemical, pharmacological, biological, biochemical and medical arts.

In certain embodiments, the pharmaceutical composition is frozen, and the method described above further comprises defrosting the frozen pharmaceutical composition prior to use.

In certain embodiments, the stem cells are autologous to the subject afflicted with the debilitating condition.

Contacting functional mitochondria with stem cells autologous to the subject afflicted with a debilitating condition results in rejuvenation/revitalization of the stem cells.

In some embodiments, the methods described above in various embodiments thereof further comprises expanding the stem cells of the first composition by culturing said stem cells in a proliferation medium capable of expanding stem cells. In other embodiments, the method further comprises expanding the mitochondrially-enriched stem cells of the fourth composition by culturing said cells in a culture or proliferation medium capable of expanding stem cells. As used throughout this application, the term “culture or proliferation medium” is a fluid medium such as cell culture media, cell growth media, buffer which provides sustenance to the cells. As used throughout this application, and in the claims the term “pharmaceutical composition” comprises a fluid carrier such as cell culture media, cell growth media, buffer which provides sustenance to the cells.

In certain embodiments, administration of the stem cells rejuvenated by functional mitochondria in the subject afflicted with debilitating effects can diminish these effects. In some embodiments, administration of the rejuvenated stem cells can restore the organization and distribution of epithelial cells in the intestinal villi of the subject afflicted with a debilitating condition. In other embodiments, administration of the rejuvenated stem cells can restore the activity of epithelial stem cells in the intestinal crypts of the subject. In further embodiments, administration of the rejuvenated stem cells can restore dermal thickness in the subject. In yet further embodiments, administration of the rejuvenated stem cells can restore hair follicle activity in the subject. In additional embodiments, the administration of the rejuvenated stem cells can restore wound healing activity in the dermal tissue of a subject. According to some embodiments, stem cells enriched with functional mitochondria can rejuvenate blood precursor cells in an autologous hematopoietic stem cell graft. According to other embodiments, stem cells enriched with functional mitochondria can rejuvenate blood precursor cells in an allogeneic hematopoietic stem cell graft. According to yet other embodiments, stem cells enriched with functional mitochondria can rejuvenate dermal or intestinal epithelial precursor cells. In additional embodiments, the administration of the rejuvenated stem cells can restore pancreatic function of β-cells in a subject. According to some embodiments, stem cells enriched with functional mitochondria can rejuvenate liver hepatocytes. According to other embodiments, stem cells enriched with functional mitochondria can retard kidney function deterioration. According to yet other embodiments, stem cells enriched with functional mitochondria can diminish macular degeneration.

In certain embodiments, the stem cells are allogeneic to the subject afflicted with the debilitating condition. The term “allogeneic to the subject”, “from a donor” and “from a healthy donor” are used herein interchangeably and refer to the stem cells or mitochondria being from a different donor individual. If possible, the donor stem cells preferably are HLA matched to the cells of the patient or at least partially HLA matched. According to certain embodiments, the donor is matched to the patient according to identification of a specific mitochondrial DNA haplogroup.

The term “HLA-matched” as used herein refers to the desire that the patient and the donor of the stem cells be as closely HLA-matched as possible, at least to the degree in which the patient does not develop an acute immune response against the stem cells of the donor. The prevention and/or therapy of such an immune response may be achieved with or without acute or chronic use of immune-suppressors. In certain embodiments, the stem cells from the donor are HLA-matched to the patient to a degree wherein the patient does not reject the stem cells.

In certain embodiment, the patient is further treated by an immunosuppressive therapy to prevent immune rejection of the stem cells graft.

In certain embodiments the mitochondria are from identical haplogroups.

In other embodiments the mitochondria are from different haplogroups.

In certain embodiments, the method described above further comprises a preceding step of administering to the subject a pre-transplant conditioning agent prior to the administration of the pharmaceutical composition. The term “pre-transplant conditioning agent” as used herein refers to any agent capable of killing bone-marrow cells within the bone-marrow of a human subject. In certain embodiments, the pre-transplant conditioning agent is Busulfan.

In certain embodiments, the pharmaceutical composition is administered systemically. In certain embodiments, the administration of the pharmaceutical composition to a subject is by a route selected from the group consisting of intravenous, intraarterial, intramuscular, subcutaneous, intravitreal, and direct injection into a tissue or an organ. Each possibility represents a separate embodiment of the invention. According to certain embodiments, the pharmaceutical composition is injected directly to tissues and organs affected by the debilitating conditions of the present invention. Specific tissues or organs that are known to show impaired function associated with a decline in mitochondrial quality and activity, include but are not limited to: eyes, kidneys, liver, pancreas, brain, and heart.

In certain embodiments, the functional mitochondria are obtained from a human cell or a human tissue selected from the group consisting of placenta, placental cells grown in culture, and blood cells. Each possibility represents a separate embodiment of the invention.

According to certain embodiments, the functional mitochondria have undergone a freeze-thaw cycle. Without wishing to be bound by any theory or mechanism, mitochondria that have undergone a freeze-thaw cycle demonstrate a comparable oxygen consumption rate following thawing, as compared to control mitochondria that have not undergone a freeze-thaw cycle.

According to some embodiments, the freeze-thaw cycle comprises freezing said functional mitochondria for at least 24 hours prior to thawing. According to other embodiments, the freeze-thaw cycle comprises freezing said functional mitochondria for at least 1 month prior to thawing, several months prior to thawing or longer. Each possibility represents a separate embodiment of the present invention. According to another embodiment, the oxygen consumption of the functional mitochondria after the freeze-thaw cycle is equal or higher than the oxygen consumption of the functional mitochondria prior to the freeze-thaw cycle.

As used herein, the term “freeze-thaw cycle” refers to freezing of the functional mitochondria to a temperature below 0° C., maintaining the mitochondria in a temperature below 0° C. for a defined period of time and thawing the mitochondria to room temperature or body temperature or any temperature above 0° C. which enables treatment of the stem cells with the mitochondria. Each possibility represents a separate embodiment of the present invention. The term “room temperature”, as used herein typically refers to a temperature of between 18° C. and 25° C. The term “body temperature”, as used herein, refers to a temperature of between 35.5° C. and 37.5° C., preferably 37° C. In another embodiment, mitochondria that have undergone a freeze-thaw cycle are functional mitochondria.

In another embodiment, the mitochondria that have undergone a freeze-thaw cycle were frozen at a temperature of −70° C. or lower. In another embodiment, the mitochondria that have undergone a freeze-thaw cycle were frozen at a temperature of −20° C. or lower. In another embodiment, the mitochondria that have undergone a freeze-thaw cycle were frozen at a temperature of −4° C. or lower. According to another embodiment, freezing of the mitochondria is gradual. According to some embodiment, freezing of mitochondria is through flash-freezing. As used herein, the term “flash-freezing” refers to rapidly freezing the mitochondria by subjecting them to cryogenic temperatures.

In another embodiment, the mitochondria that underwent a freeze-thaw cycle were frozen for at least 30 minutes prior to thawing. According to another embodiment, the freeze-thaw cycle comprises freezing the functional mitochondria for at least 30, 60, 90, 120, 180, 210 minutes prior to thawing. Each possibility represents a separate embodiment of the present invention. In another embodiment, the mitochondria that have undergone a freeze-thaw cycle were frozen for at least 1, 2, 3, 4, 5, 6, 7, 8, 9, 10, 24, 48, 72, 96, or 120 hours prior to thawing. Each freezing time presents a separate embodiment of the present invention. In another embodiment, the mitochondria that have undergone a freeze-thaw cycle were frozen for at least 4, 5, 6, 7, 30, 60, 120, 365 days prior to thawing. Each freezing time presents a separate embodiment of the present invention. According to another embodiment, the freeze-thaw cycle comprises freezing the functional mitochondria for at least 1, 2, 3 weeks prior to thawing. Each possibility represents a separate embodiment of the present invention. According to another embodiment, the freeze-thaw cycle comprises freezing the functional mitochondria for at least 1, 2, 3, 4, 5, 6 months prior to thawing. Each possibility represents a separate embodiment of the present invention.

In another embodiment, the mitochondria that have undergone a freeze-thaw cycle were frozen at −70° C. for at least 30 minutes prior to thawing. Without wishing to be bound by any theory or mechanism, the possibility to freeze mitochondria and thaw them after a long period enables easy storage and use of the mitochondria with reproducible results even after a long period of storage.

According to certain embodiment, thawing is at room temperature. In another embodiment, thawing is at body temperature. According to another embodiment, thawing is at a temperature which enables administering the mitochondria according to the methods of the invention. According to another embodiment, thawing is performed gradually.

According to another embodiment, the mitochondria that underwent a freeze-thaw cycle were frozen within a freezing buffer. According to another embodiment, the mitochondria that underwent a freeze-thaw cycle were frozen within the isolation buffer. As used herein, the term “isolation buffer” refers to a buffer in which the mitochondria of the invention have been isolated. In a non-limiting example, the isolation buffer is a sucrose buffer. Without wishing to be bound by any mechanism or theory, freezing mitochondria within the isolation buffer saves time and isolation steps, as there is no need to replace the isolation buffer with a freezing buffer prior to freezing or to replace the freezing buffer upon thawing.

According to another embodiment, the freezing buffer comprises a cryoprotectant. According to some embodiments, the cryoprotectant is a saccharide, an oligosaccharide or a polysaccharide. Each possibility represents a separate embodiment of the present invention. According to another embodiment, the saccharide concentration in the freezing buffer is a sufficient saccharide concentration which acts to preserve mitochondrial function. According to another embodiment, the isolation buffer comprises a saccharide. According to another embodiment, the saccharide concentration in the isolation buffer is a sufficient saccharide concentration which acts to preserve mitochondrial function. According to another embodiment, the saccharide is sucrose.

In certain embodiments, the method further comprises the preceding steps of (a) freezing the human stem cells enriched with healthy functional human exogenous mitochondria, (b) thawing the human stem cells enriched with healthy functional human exogenous mitochondria, and (c) administering the human stem cells enriched with healthy functional human exogenous mitochondria to the patient.

In certain embodiments, the healthy functional exogenous mitochondria constitute at least 3% of the total mitochondria in the mitochondrially-enriched cell. In certain embodiments, the healthy functional exogenous mitochondria constitute at least 10% of the total mitochondria in the mitochondrially-enriched cell. In some embodiments, the healthy functional exogenous mitochondria constitute at least about 3%, 5%, 10%, 15%, 20%, 25% or 30% of the total mitochondria in the mitochondrially-enriched cell. Each possibility represents a separate embodiment of the present invention.

The extent of enrichment of the stem cells with functional mitochondria may be determined by functional and/or enzymatic assays, including but not limited to rate of oxygen (O₂) consumption, content or activity level of citrate synthase, rate of adenosine triphosphate (ATP) production. In the alternative the enrichment of the stem cells with healthy donor mitochondria may be confirmed by the detection of mitochondrial DNA of the donor. According to some embodiments, the extent of enrichment of the stem cells with functional mitochondria may be determined by the level of change in heteroplasmy and/or by the copy number of mtDNA per cell. Each possibility represents a separate embodiment of the present invention.

TMRM (tetramethylrhodamine methyl ester) or the related TMRE (tetramethylrhodamine ethyl ester) are cell-permeant fluorogenic dyes commonly used to assess mitochondrial function in living cells, by identifying changes in mitochondrial membrane potential. According to some embodiments, the level of enrichment can be determined by staining with TMRE or TMRM.

According to some embodiments, the intactness of a mitochondrial membrane may be determined by any method known in the art. In a non-limiting example, intactness of a mitochondrial membrane is measured using the tetramethylrhodamine methyl ester (TMRM) or the tetramethylrhodamine ethyl ester (TMRE) fluorescent probes. Each possibility represents a separate embodiment of the present invention. Mitochondria that were observed under a microscope and show TMRM or TMRE staining have an intact mitochondrial outer membrane. As used herein, the term “a mitochondrial membrane” refers to a mitochondrial membrane selected from the group consisting of the mitochondrial inner membrane, the mitochondrial outer membrane, and both.

In certain embodiments, the level of mitochondrial enrichment in the mitochondrially-enriched human stem cells is determined by sequencing at least a statistically-representative portion of total mitochondrial DNA in the cells and determining the relative levels of host/endogenous mitochondrial DNA and exogenous mitochondrial DNA. In certain embodiments, the level of mitochondrial enrichment in the mitochondrially-enriched human stem cells is determined by single nucleotide polymorphism (SNP) analysis. In certain embodiments, the largest mitochondrial population and/or the largest mitochondrial DNA population is the host/endogenous mitochondrial population and/or the host/endogenous mitochondrial DNA population; and/or the second-largest mitochondrial population and/or the second-largest mitochondrial DNA population is the exogenous mitochondrial population and/or the exogenous mitochondrial DNA population. Each possibility represents a separate embodiment of the invention.

According to certain embodiments, the enrichment of the stem cells with healthy functional mitochondria may be determined by conventional assays that are recognized in the art. In certain embodiments, the level of mitochondrial enrichment in the mitochondrially-enriched human stem cells is determined by (i) the levels of host/endogenous mitochondrial DNA and exogenous mitochondrial DNA; (ii) the level of mitochondrial proteins selected from the group consisting of citrate synthase (CS), cytochrome C oxidase (COX1), succinate dehydrogenase complex flavoprotein subunit A (SDHA) and any combination thereof; (iii) the level of CS activity; or (iv) any combination of (i), (ii) and (iii). Each possibility represents a separate embodiment of the invention.

In certain embodiments, the level of mitochondrial enrichment in the mitochondrially-enriched human stem cells is determined by at least one of: (i) the levels of host mitochondrial DNA and exogenous mitochondrial DNA in case of allogeneic mitochondria; (ii) the level of citrate synthase activity; (iii) the level of succinate dehydrogenase complex flavoprotein subunit A (SDHA) or cytochrome C oxidase (COX1); (iv) the rate of oxygen (O₂) consumption; (v) the rate of adenosine triphosphate (ATP) production or (vi) any combination thereof. Each possibility represents a separate embodiment of the present invention. Methods for measuring these various parameters are well known in the art.

In some aspects, the present invention provides a pharmaceutical composition comprising human stem cells enriched with healthy functional mitochondria for use in treating or diminishing debilitating effects of conditions in a subject, wherein the debilitating effects of conditions are selected from the group consisting, but not limited to, aging, age-related diseases and the sequel of anti-cancer treatments.

In some embodiments, the present invention provides a method for treating or diminishing debilitating effects of conditions in a subject, comprising administering a pharmaceutical composition comprising human stem cells enriched with healthy functional mitochondria to the subject, wherein the debilitating effects of conditions are selected from the group consisting, but not limited to aging, age-related diseases and the sequel of anti-cancer treatments. In specific embodiments, the anti-cancer treatments are selected from the group consisting of radiation, chemotherapy, immunotherapy with monoclonal antibodies or any combination thereof.

According to certain embodiments, the healthy functional mitochondria are isolated from a donor selected from a specific mitochondria haplogroup, in accordance with the debilitating condition of the subject. For example, for the aging subject, administration of stem cells enriched with functional mitochondria from the J mitochondrial haplogroup is suitable due to its association with longevity and lower blood pressure (De Benedictis et al., FASEB J. 1999; 13(12):1532-6; Rea et al., AGE 2013; 34(4):1445-56). H and N haplogroups are associated with better muscle functionality and strength (Larsen et al., Biochim Biophys Acta. 2014; 1837(2):226-31; Fuku et al., Int J Sports Med. 2012; 33(5):410-4). D4b haplogroup may be protective against stroke (Yang et al., Mol Genet Genomics. 2014; 289(6):1241-6), K, U, H and V haplogroups may confer protection against cognitive impairment (Colicino et al., Environ Health. 2014; 13(1):42) and R haplogroup has been shown to confer better prognosis of recovery from septic encephalopathy (Yang et al., Intensive Care Med. 2011; 37(10):1613-9). Haplogroup N9a confers resistance to diabetes (Fuku et al., Am J Hum Genet. 2007; 80(3):407-15) and to metabolic syndrome (Tanaka et al., Diabetes 2007; 56(2): 518-21). H haplogroup is protective against developing eye diseases including age-related macular degeneration (AMD) (Mueller et al., PloS one 2012; 7(2): e30874).

According to certain embodiments, the stem cells of the first composition are from a donor selected from a specific mitochondrial haplogroup, in accordance with the debilitating condition of the subject. For example, the subject afflicted with debilitating effects of anti-cancer treatments, the J, K2, and U haplogroups may be considered, since they were shown to be better donors for allogeneic hematopoietic stem cell transplantation, eliciting less GVHD and/or relapse (Ross et al. Biol Blood Marrow Transplant 2015; 21:81-88).

The term “haplogroup” as used herein refers to a genetic population group of people who share a common ancestor on the matriline. Mitochondrial haplogroup is determined by sequencing.

In certain cases we might want to match haplotypes between donor and acceptor.

The term “about” as used herein means a range of 10% below to 10% above the indicated integer, number or amount. For example, the phrase “about 1*10⁵” means “1.1*10⁵ to 9*10⁴”.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

The following examples are presented to provide a more complete understanding of the invention. The specific techniques, conditions, materials, proportions and reported data set forth to illustrate the principles of the invention are exemplary and should not be construed as limiting the scope of the invention.

### EXAMPLES

### Example 1. Isolated Human Mitochondria: Preparation and Cryopreservation

Mitochondria can be isolated and preserved as disclosed previously in WO 2013/035101 and WO 2016/135723.

The following are exemplary protocols used for isolation of mitochondria from peripheral blood cells (MNV-BLD) and enrichment of CD34⁺ cells (MNV-BM-BLD):

### First Stage—MNV-BLD Production:

The buffy coat is isolated from peripheral blood (500 mL) obtained from the patient or donated by a donor. The buffy coat is then layered on top of Lymphoprep™ and centrifuged. The white cells (buffy coat on top of Lymphoprep™) are collected, and then centrifuged. The cell pellet (lymphocytes) is washed and cell pellet is frozen and suspended in ice-cold 250 mM sucrose buffer solution (250 mM sucrose, 10 mM Tris, 1 mM EDTA) pH=7.4. The cell suspension is collected and passed through a 30G needle 3 times, following by homogenization. The homogenate is centrifuged. The supernatant is collected and kept on ice, and the pellet is washed with sucrose solution, homogenized and centrifuged. The second supernatant from the washed pellet is collected and combined with the previous supernatant. The combined supernatant is filtered through a 5 μm filter and centrifuged at 8000 g. Pellets are washed with sucrose solution and re-suspended in 1 ml cold 250 mM Sucrose buffer solution pH=7.4. The resulting mitochondria solution (denoted herein as MNV-BLD) is cryopreserved in a vapor-phase nitrogen tank until use.

### Second Stage—MNV-BM-BLD Generation:

Patient's or Donor's CD34⁺ cells are isolated from blood collected via leukapheresis using the CliniMACS™ system, following mobilization of bone marrow cells to the peripheral blood. The CD34⁺ cells pellet is suspended in 4.5% HSA in 0.9% NaCl solution to a final concentration of 1×10⁶ cells/ml. MNV-BLD (mitochondria suspension) is thawed at room temperature and added to the CD34⁺ cells at 4.4 citrate synthase (CS) activity units per ml of cell suspension (1×10⁶ cells). MNV-BLD and CD34⁺ cells are mixed in 2 mL tubes, and centrifuged at 7000 g for 5 minutes at 4° C. After centrifugation, the cells are suspended with the same 4.5% HSA in 0.9% NaCl solution, combined and seeded in a flask and incubated at room temperature for 24 hours. Following incubation, enriched CD34⁺ cells are washed twice with 4.5% HSA solution and centrifuged at 300 g for 10 min. The cell pellet is re-suspended in 100 ml 4.5% HSA in 0.9% NaCl, and filled into an infusion bag.

### Example 2. Isolated Mitochondria can Enter Fibroblast Cells

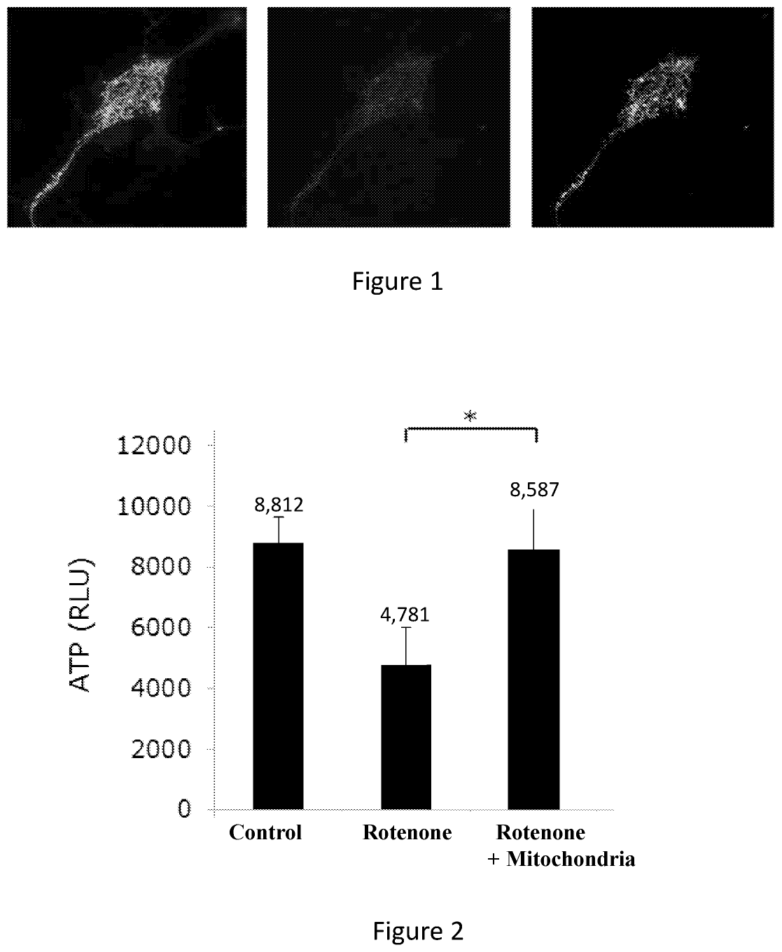

Mouse fibroblast cells (10⁴, 3T3) expressing green fluorescent protein (GFP) in their mitochondria (left panel) were incubated for 24 hours with red fluorescent protein (RFP)-labeled mitochondria isolated from mouse fibroblasts (3T3) expressing RFP in their mitochondria (middle panel). Fluorescent confocal microscopy was used to identify fibroblasts labeled with both GFP and RFP, which appear yellow (right panel) (FIG. 1), as previously described in WO 2016/135723.

The results demonstrated in FIG. 1 indicate that mitochondria can enter fibroblast cells.

### Example 3. Mitochondria Increase ATP Production in Cells with Inhibited Mitochondrial Activity

Mouse fibroblast cells (10⁴, 3T3) were either not treated (control) or treated with 0.5 μM Rotenone (Rotenone, mitochondrial complex I irreversible inhibitor, CAS number 83-79-4) for 4 hours, washed, and further treated with 0.02 mg/ml mouse placental mitochondria (Rotenone+Mitochondria) for 3 hours. The cells were washed and ATP level was determined using the Perkin Elmer ATPlite kit (FIG. 2), as previously shown in WO 2016/135723. As seen in FIG. 2, the production of ATP was completely rescued in cells incubated with mitochondria compared to control.

The results demonstrated in FIG. 2 clearly indicate that while Rotenone alone decreased ATP levels by about 50%, the addition of mitochondria was capable of substantially cancelling the inhibitory effect of Rotenone, reaching the ATP levels of the control cells. The experiment provides evidence of the capability of mitochondria to increase mitochondrial ATP production in cells with impaired or compromised mitochondrial activity.

### Example 4. Mitochondria can Enter Murine Bone Marrow Cells

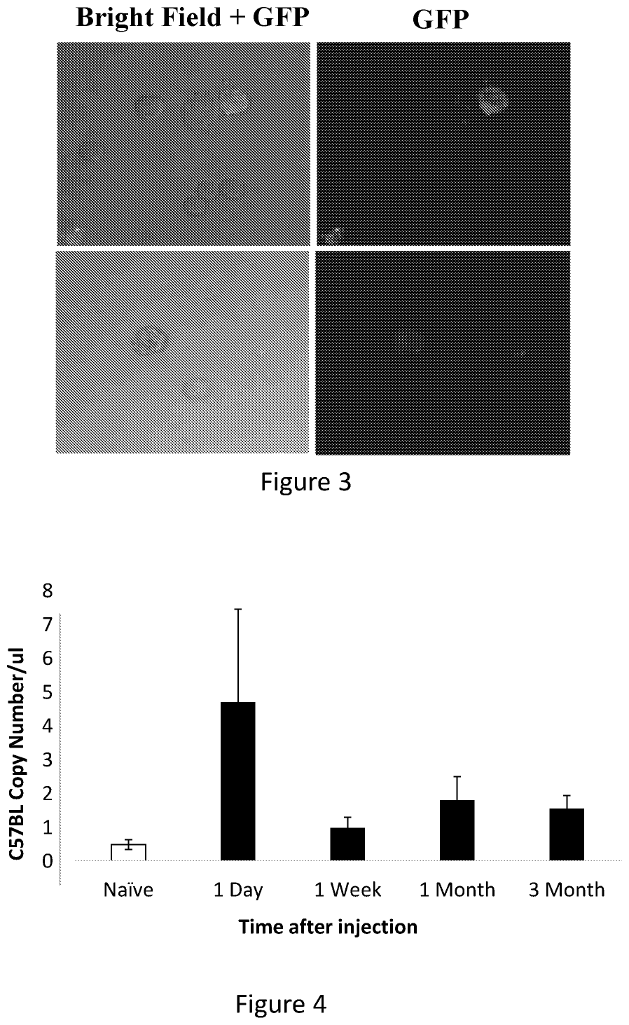

Mouse bone marrow cells (10⁵) were incubated for 24 hours with GFP-labeled mitochondria, isolated from mouse melanoma cells. Fluorescence confocal microscopy was used to identify GFP-labeled mitochondria inside the bone marrow cells (FIG. 3), as previously described in WO 2016/135723.

The results demonstrated in FIG. 3 indicate that mitochondria can enter bone marrow cells.

Bone marrow cells from wild type (ICR) and mutated mitochondria (FVB/N, carries a mutation in ATP8) mice were incubated in DMEM for 24 hours at 37° C. and 5% CO₂ atmosphere with isolated mitochondria of different origins in order to increase their mitochondrial content and activity. Table 1 describes representative results of the mitochondrial augmentation process, determined by the relative increase in CS activity of the cells after the process compared to the CS activity of the cells before the process.

TABLE 1

| | | | Relative |
|---|---|---|---|
| | | | increase |
| | | CS activity of | in CS |
| | Origin of | mitochondria/ | activity |
| Origin of cells | mitochondria | number of cells | of cells |
| ICR Mouse—Isolated | Human | 4.4 U CS/1 × | +41% |
| from whole bone marrow | mitochondria | 10\hat{ }6 Cells | |
| FVB/N Mouse—Isolated | C57BL placental | 4.4 U CS/1 × | +70% |
| from whole bone marrow | mitochondria | 10\hat{ }6 Cells | |
| FVB/N Mouse—Isolated | C57BL liver | 4.4 U CS/1 × | +25% |
| from whole bone marrow | mitochondria | 10\hat{ }6 Cells | |

In order to examine in vivo the effect of mitochondrial augmentation therapy, FVB/N bone marrow cells (1×10⁶) enriched with 4.4 mUnits CS activity of C57/BL placental mitochondria, were IV injected to FVB/N mice. Bone marrow were collected from mice 1 day, 1 week, 1 month and 3 months after the treatment and the level of WT mtDNA were detected using dPCR. As can be seen in FIG. 4, significant amount of WT mtDNA was detected in bone marrow 1 day post treatment.

### Example 5. Mitochondria Enter Bone Marrow Cells in a Concentration-Dependent Manner

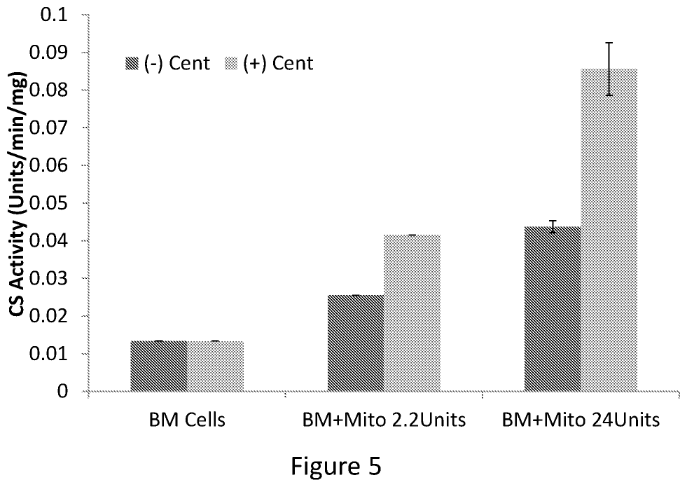

Mouse bone marrow cells (10⁶) were untreated or incubated for 15 hours with different amounts of GFP-labeled mitochondria isolated from mouse melanoma cells. Before plating the cells, mitochondria were mixed with the cells and either left to stand for 5 minutes at room temperature ((−) Cent) or centrifuged for 5 minutes at 8,000 g at 4° C. ((+) Cent). The cells were then plated in 24 wells (10⁶ cells/well). After 15 hours of incubation, the cells were washed twice to remove any mitochondria that did not enter the cells. Citrate synthase activity was determined using the CS0720 Sigma kit (FIG. 5), as previously described in WO 2016/135723. The CS activity levels measured under the conditions specified above are summarized in Table 2.

TABLE 2

| | | | (+) Cent, | (−) Cent, |
|---|---|---|---|---|
| | (+) Cent | (−) Cent | normalized | normalized |
| Cells | 0.013368 | 0.013368 | 1 | 1 |
| Cells + Mitochondria | 0.041512 | 0.025473 | 3.1 | 1.9 |
| (2.2 units) | | | | |
| Cells + Mitochondria | 0.085606 | 0.04373 | 6.4 | 3.2 |
| (24 units) | | | | |

The results demonstrated in FIG. 5 indicate that added mitochondria increase cellular CS activity in a dose-dependent manner, and that increasing the concentration and therefore presumably the contact between the mitochondria and cells, e.g. by centrifugation, resulted in a further increase in CS activity.

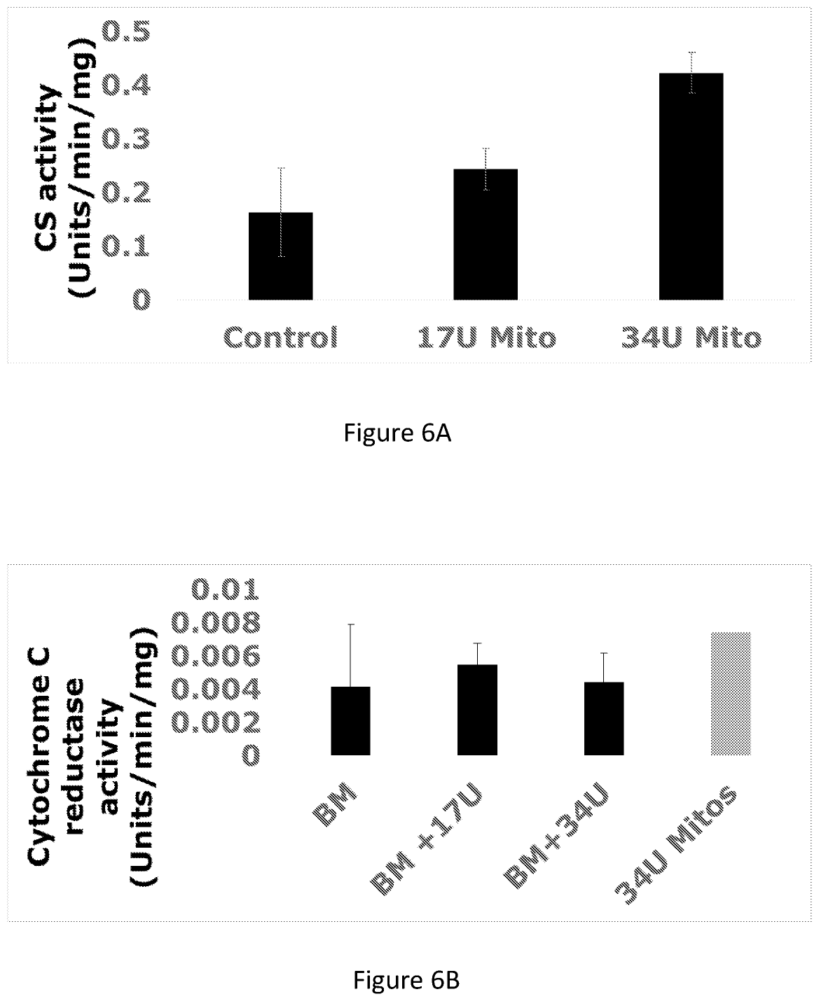

Mouse bone-marrow cells (10⁶) were untreated or incubated for 24 hours with GFP-labeled mitochondria isolated from mouse melanoma cells (17U or 34U, indicating the level of citrate synthase activity as a marker for mitochondria content). The cells were mixed with mitochondria, centrifuged at 8000 g and re-suspended. After 24 hour incubation, the cells were washed twice with PBS and the level of citrate synthase (CS) activity (FIG. 6A) and cytochrome c reductase activity (FIG. 6B) were measured using the CS0720 and CYOIOO kits (Sigma), respectively, as previously described in WO 2016/135723.

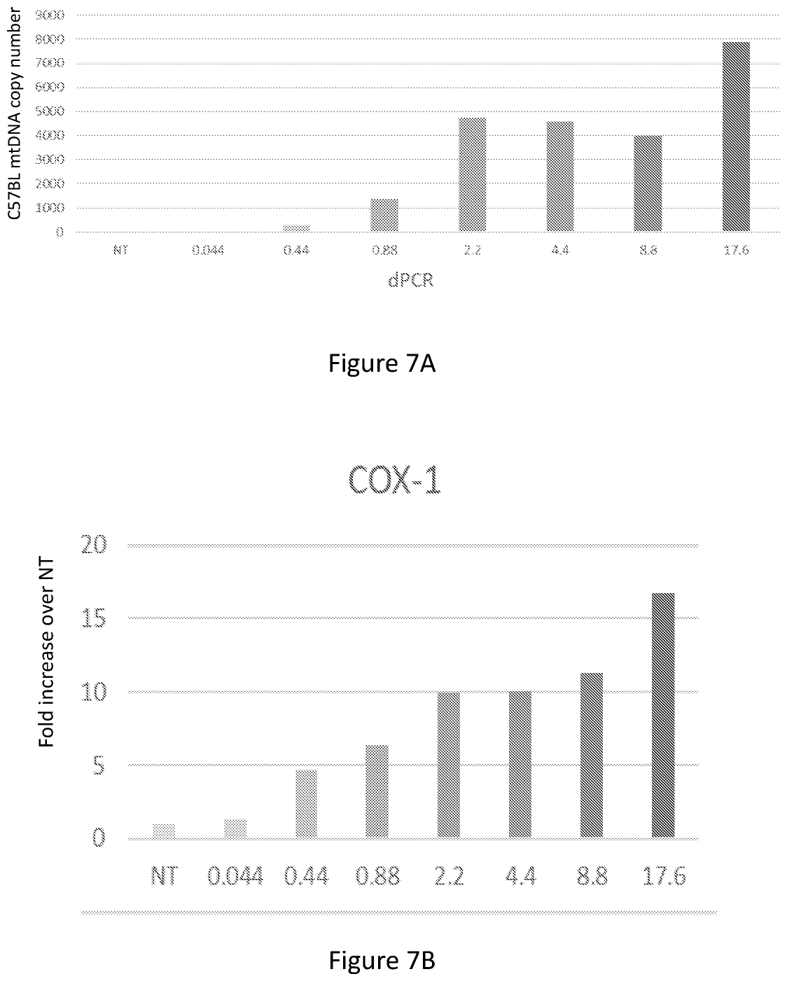

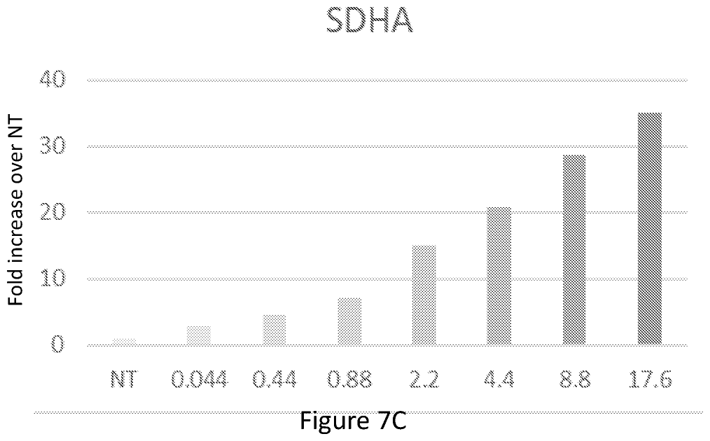

FVB/N bone marrow cells (carrying a mutation in mtDNA ATP8) were incubated with C57/BL wild-type (WT) mitochondria isolated from placenta in various doses (0.044, 0.44, 0.88, 2.2, 4.4, 8.8, 17.6 mUnits CS activity per 1 M cells in 1 mL). As can be seen in FIG. 7A, dPCR using WT specific sequences showed an increase in WT mtDNA in a dose-dependent manner for most dosages. The enriched cells also showed a dose-dependent increase in content of mtDNA encoded (COX1) (FIG. 7B) and nuclear encoded (SDHA) (FIG. 7C).

### Example 6. Mitochondria can Enter Human Bone Marrow Cells

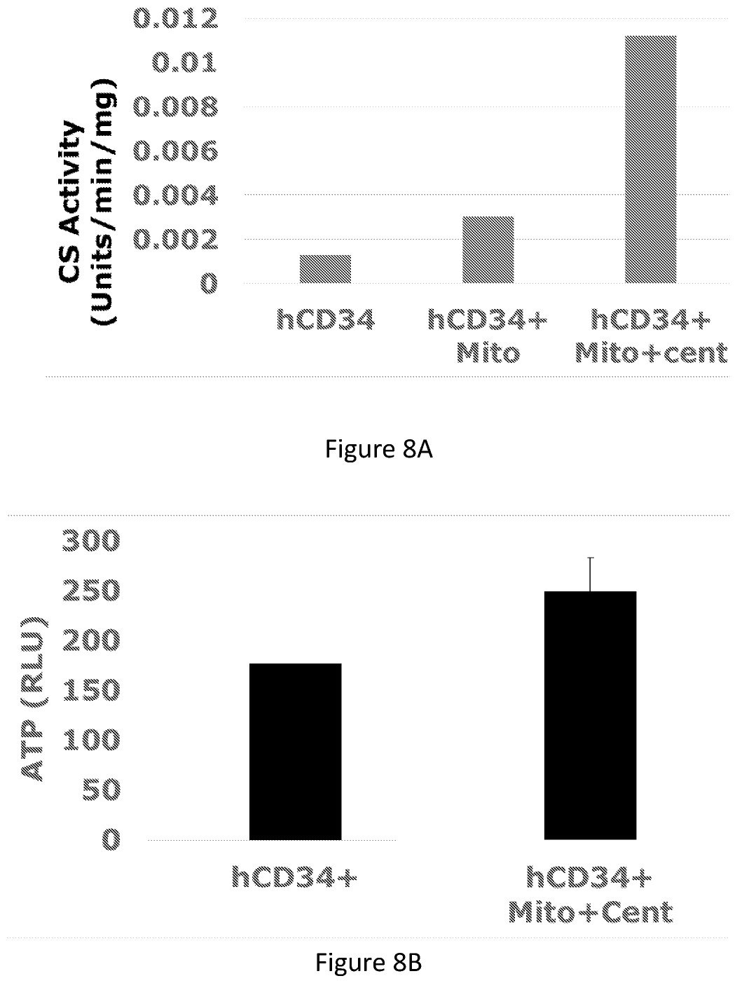

Human CD34⁺ cells (1.4*10⁵, ATCC PCS-800-012) were untreated or incubated for 20 hours with GFP-labeled mitochondria isolated from human placental cells. Before plating the cells, mitochondria were mixed with the cells, centrifuged at 8000 g and re-suspended. After incubation, the cells were washed twice with PBS and CS activity was measured using the CS0720 Sigma kit (FIG. 8A). ATP content was measured using ATPlite (Perkin Elmer) (FIG. 8B). The CS activity levels (FIG. 8A) measured under the conditions specified above are summarized in Table 3.

TABLE 3

| | | | (+) Cent, | (−) Cent, |
|---|---|---|---|---|
| | (+) Cent | (−) Cent | normalized | normalized |
| Cells | | 0.001286445 | | 1 |
| Cells + | | 0.003003348 | | 2.33 |
| Mitochondria | | | | |
| Cells + | 0.011202225 | | 8.7 | |
| Mitochondria + | | | | |
| Centrifugation | | | | |

The results demonstrated in FIG. 8 (see Table 3) clearly indicate that the mitochondrial content of human bone marrow cells may be increased many fold by interaction and co-incubation with isolated human mitochondria, to an extent beyond the capabilities of either human or murine fibroblasts or murine bone marrow cells.

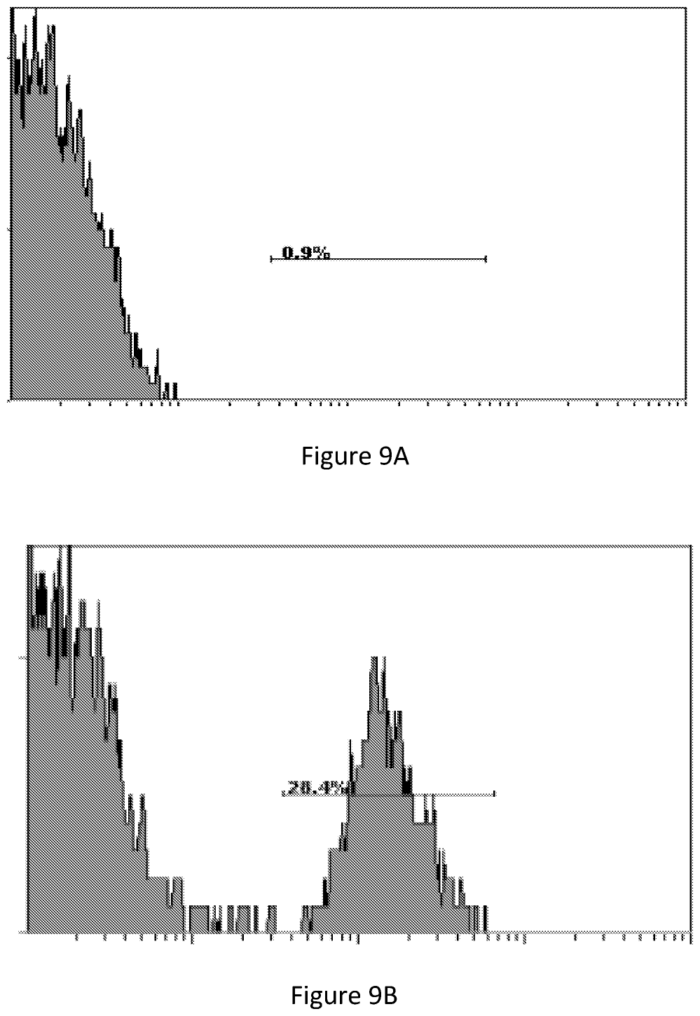

The cell populations depict in FIG. 8B were further evaluated by FACS analysis. While in the CD34⁺ cells not incubated with GFP-labeled mitochondria only a minor portion (0.9%) of the cells were fluorescent (FIG. 9A), the CD34⁺ cells incubated with GFP-labeled mitochondria after centrifugation were substantially fluorescent (28.4%) (FIG. 9B), as previously shown in WO 2016/135723.

### Example 7. Mitochondria can Enter Human CD34⁺ Bone Marrow Cells

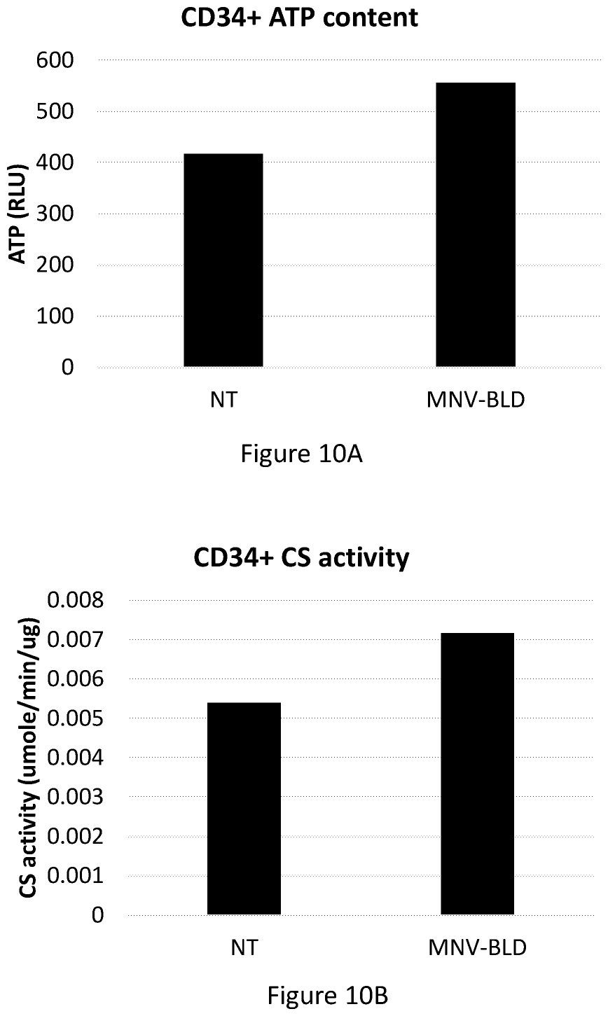

Human CD34⁺ cells of a healthy donor treated with GCS-F were obtained by apheresis, purified using CliniMACS system and frozen. The cells were thawed and treated with blood derived mitochondria (MNV-BLD) (4.4U mitochondrial CS activity per 1×10⁶ cells), or not treated (NT), centrifuged at 8000 g and incubated for 24 h. Cells were then washed with PBS and CS activity (FIG. 10A) and ATP content (FIG. 10B) were measured (using the CS0720 Sigma kit and ATPlite Perkin Elmer, respectively).

CD34⁺ cells treated with blood derived mitochondria showed a remarkable increase in mitochondrial activity, as measured by CS activity (FIG. 10A) and ATP content (FIG. 10B).

CD34⁺ cells from healthy donors were treated with Mitotracker Orange (MTO) and washed prior to MAT, using mitochondria isolated from HeLa-TurboGFP-Mitochondria cells (CellTrend GmbH). Cells were fixed with 2% PFA for 10 minutes and fixed with DAPI. Cells were scanned using confocal microscope equipped with a 60×/1.42 oil immersion objective.

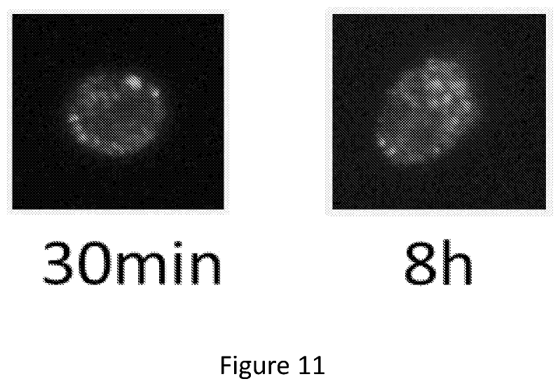

As can be seen in FIG. 11, exogenous mitochondria enter CD34⁺ cell as rapidly as 0.5 hour after MAT (bright, almost white, spots inside the cell), and continues for the tested 8 and 24 hours.

### Example 8. Culturing CD34⁺ Cells in Room Temperature with Saline Improves their Viability

CD34⁺ cells were untreated (NT) or incubated with blood derived mitochondria (MNV-BLD). The cells were cultured at room temperature (RT) or 37° C. in culture medium (CellGro™) or saline (Zenalb™) with 4.5% human serum albumin (HSA).

The cell viability in different culture conditions is summarized in Table 4.

TABLE 4

| | % viability |
|---|---|
| CellGro ™ 37° C. NT | 55.3 |
| CellGro ™ 37° C. MNV-BLD | 59.6 |
| CellGro ™ RT NT | 72.5 |
| CellGro ™ RT MNV-BLD | 78.2 |
| Zenalb ™ RT NT | 93.9 |
| Zenalb ™ RT MNV-BLD | 94.7 |

The results demonstrated in Table 4 indicate that the CD34⁺ cells viability is improved when cultured at RT using human serum albumin in saline rather than culture medium.

### Example 9. Bone-Marrow from NSGS Mice Engrafted with Human Umbilical Cord Blood Contain More Human mtDNA 2 Month after MAT

Pearson-patient umbilical cord blood cells were incubated with 0.88 mU of human mitochondria for 24 hr, after which media was removed and cells were washed and resuspended in 4.5% HSA. The enriched cells were IV injected to NSGS mice (100,000 CD34⁺ cells per mouse).

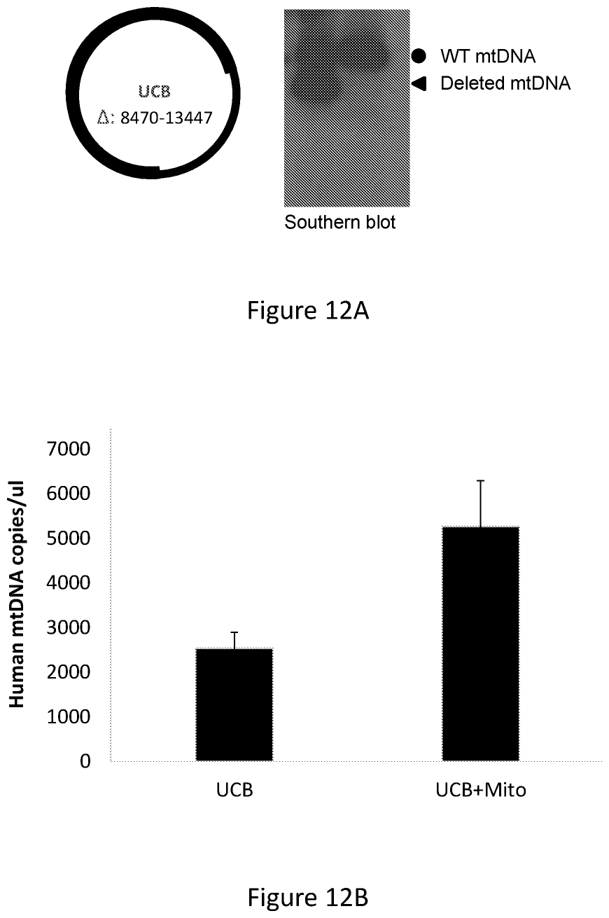

FIG. 12A is an illustration of mtDNA deletion in the Pearson-patient's cord blood cells showing 4978 kb deleted UCB mtDNA region (left) as well as a southern blot analysis showing the deletion (right).

Bone marrow was collected from mice 2 months post MAT, and copy number of non-deleted WT mtDNA was analyzed in dPCR using primers and probe identifying UCB non-deleted WT mtDNA sequences.

As can be seen in FIG. 12B, 2 months after mitochondrial augmentation therapy, bone marrow of the mice contained ˜100% more human mtDNA as compared to bone marrow of mice injected with non-augmented cord blood cells.

### Example 10. In-Vivo Safety and Bio-Distribution Animal Study

Mitochondria are introduced into bone marrow cells of control healthy mice from two different backgrounds: the source of mitochondria will be from mice with different mtDNA sequences (Jenuth J P et al., Nature Genetics, 1996, Vol. 14, pages 146-151).

Mitochondria from wild type mice (C57BL) placenta were isolated. Bone marrow cells were isolated from FVB/N mice. The mutated FVB/N bone marrow cells (10⁶) were loaded with the healthy functional C57BL mitochondria (4.4 U) and administered IV to FVB/N mice.

The steps of the method are: (1) isolating mitochondria from placenta of C57BL mice, freezing at −80° C. and defrosting, or using fresh; (2) obtaining bone marrow cells from mtDNA mutated FVB/N mice; (3) contacting the mitochondria and bone marrow cells, centrifuging at 8000 g for 5 minutes, resuspending and incubating for 24 hours; (4) washing the bone marrow cells twice with PBS and injecting into a tail vein of FVB/N mice. At various time points, e.g., after 24 hours, a week, a month and 3 months post transplantation, tissues (blood, bone marrow, lymphocytes, brain, heart, kidney, liver, lung, spleen, skeletal muscle, eye, ovary/testis) were collected and DNA extracted for further sequence analysis.

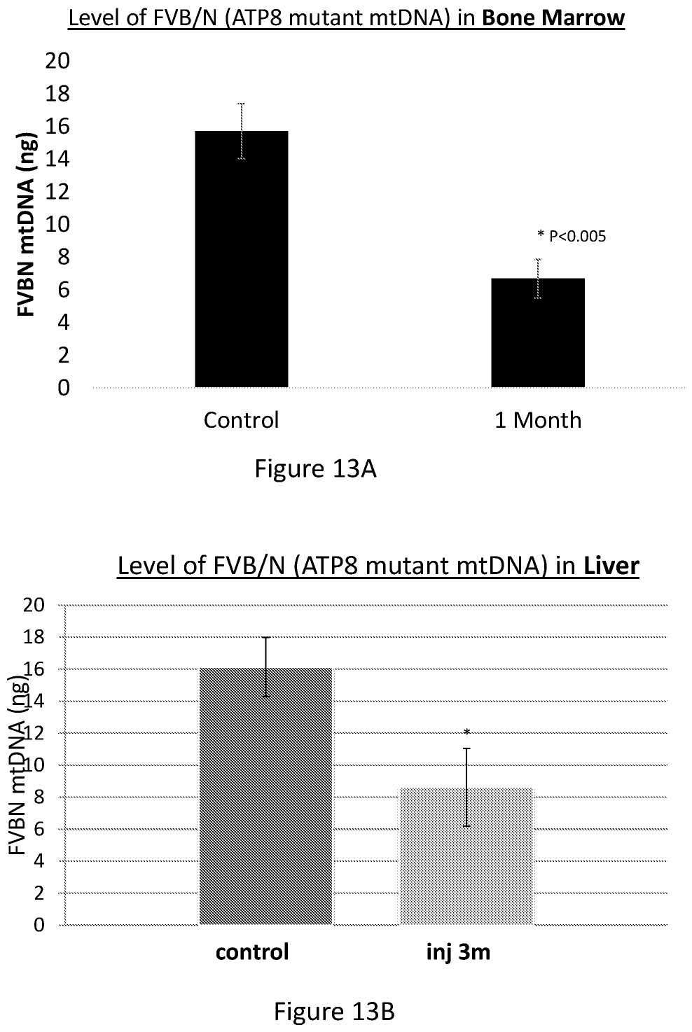

The decreased levels of FVB/N in the bone marrow 1 month after the transplantation are depicted in FIG. 13A. As seen in FIG. 13B, the mtDNA levels in livers of FVB/N mice 3 months post transplantation were also decreased.

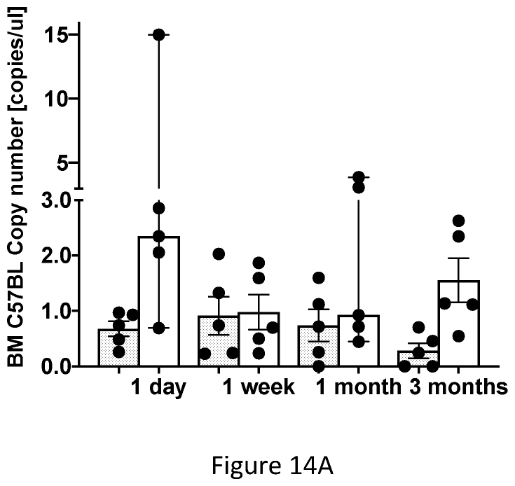

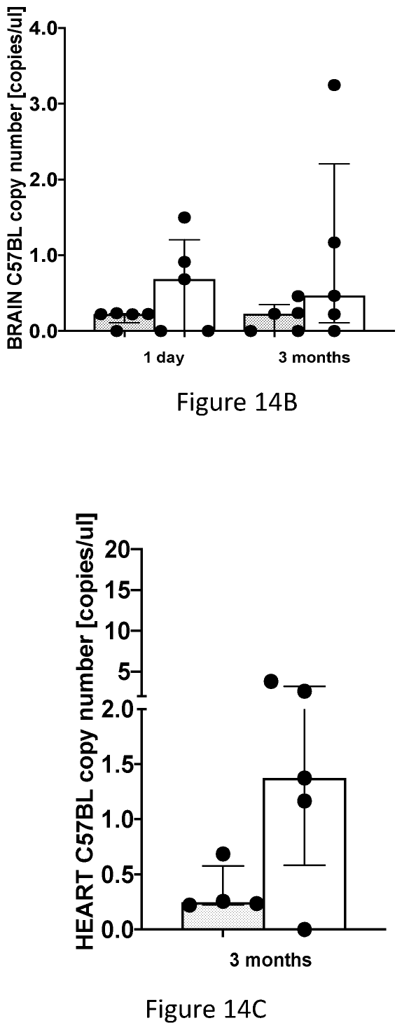

Bone marrow harvested from FVB/N females was enriched with C57BL/6 placenta mitochondria (4.4 mU CS activity per 1×10\hat{ }6 cells). Recipient mice underwent IV administration of 1 million augmented cells per animal. Digital PCR was used to detect a C57BL/6-specific SNP. FIG. 14A demonstrates the presence of C57BL/6 mtDNA in the bone marrow of FVBN mice, 1-day post-MAT, with some of the mice showing persistence up to 3 months post treatment. FIGS. 14B and 14C show the presence of C57BL/6-derived mtDNA in the hearts and brains of mice 3 months after MAT.

### Example 11. In-Vivo Pre-Clinical Animal Study: Effect of Pre-Conditioning on Engraftment of Foreign Mitochondria

Mitochondria from wild type mice (C57BL) livers were isolated. Bone marrow cells were isolated from mice with mutated mitochondria (FVB/N mice). The mutated FVB/N bone marrow cells were loaded with the healthy functional C57BL mitochondria. Untreated FVB/N mice (control), FVB/N mice administered with the enriched mitochondria, FVB/N mice treated with a chemotherapeutic agent (Busulfan) prior to administration of the enriched mitochondria and FVB/N mice that underwent total body irradiation (TBI) prior to administration of the enriched mitochondria were compared.

The steps of the method are: (1) isolating mitochondria from livers of C57BL mice, freezing at −80° C. and defrosting, or using fresh; (2) obtaining bone marrow cells from mtDNA mutated FVB/N mice; (3) contacting the mitochondria and bone marrow cells, centrifuging at 8000 g for 5 minutes, resuspending and incubating for 24 hours; (4) washing the bone marrow cells twice with PBS. (5) Busulfan administration or total body irradiation (TBI) to the intended groups. (6) injecting into a tail vein of FVB/N mice the bone marrow cells of FVB/N mice enriched with the healthy mitochondria of C57BL mice. 1 month post transplantation, tissues (blood, bone marrow, lymphocytes, brain, heart, kidney, liver, lung, spleen, pancreas, skeletal muscle, eye, ovary/testis) were collected and DNA extracted for further sequence analysis.

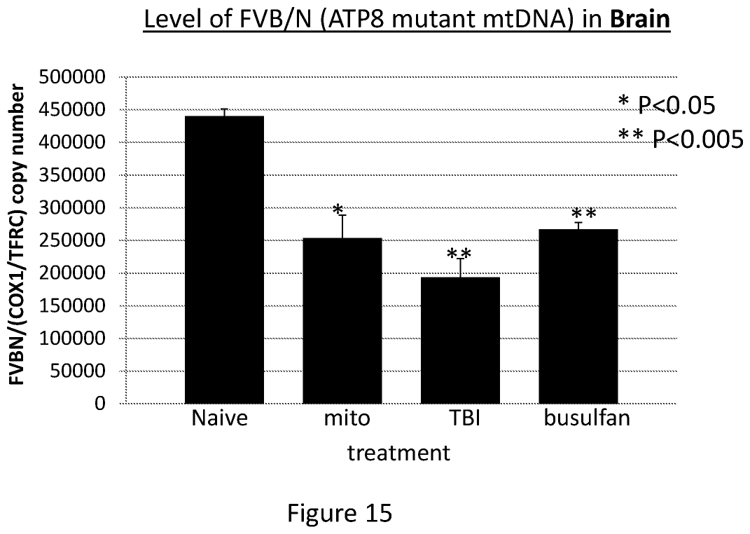

The decreased levels of FVB/N in the brains of mitochondria, TBI and Busulfan treated mice 1 month after the transplantation are depicted in FIG. 15.

### Example 12. Mitochondrial Enrichment Effect on Aging Mice

Mitochondria were isolated from term C57BL murine placenta. Bone marrow cells of 12 months old C57BL mice were obtained. Bone marrow cells enriched with mitochondria (MNV-BM-PLC, 1×106 cells), bone marrow cells alone (BM, 1×106 cells) or a control vehicle solution (VEHICLE, 4.5% Albumin in 0.9% w/v NaCl) were injected IV to the tail vein of 12 months old C57BL mice at the beginning of the experiment and again at about the age of 15 months, 18 months, 21 months. BUN blood test was performed 1, 3, 4 and 6 months post first IV injection. Open field test was performed 9 months post first IV injection. BUN blood test was performed 2, 4 and 6 months post IV injection.

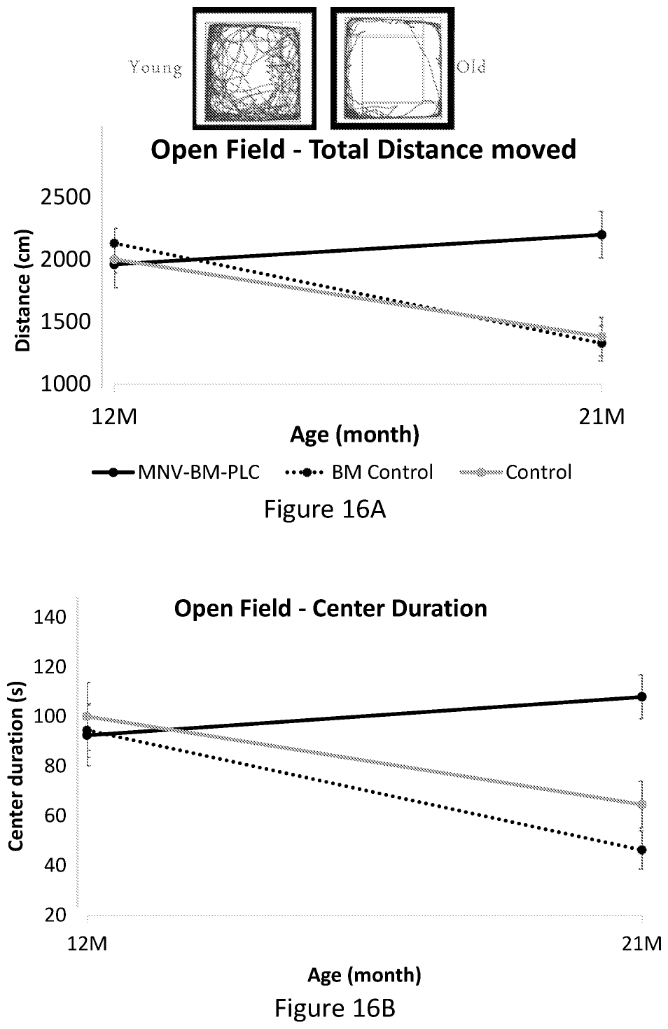

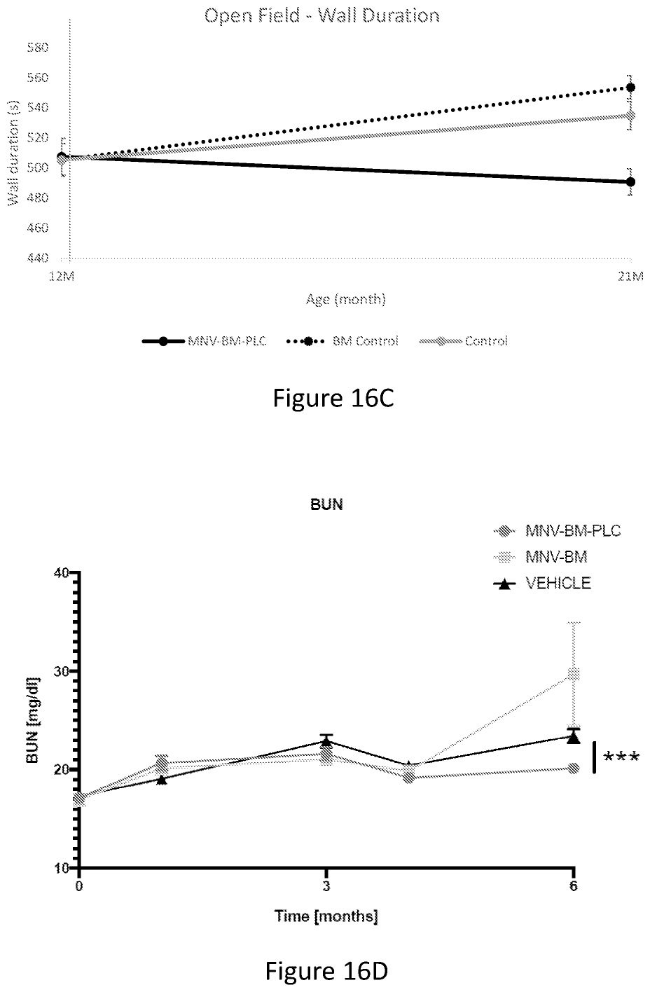

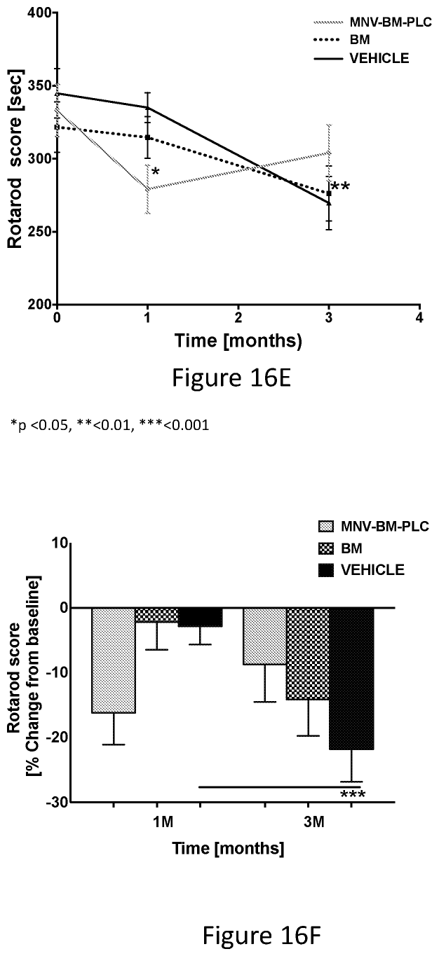

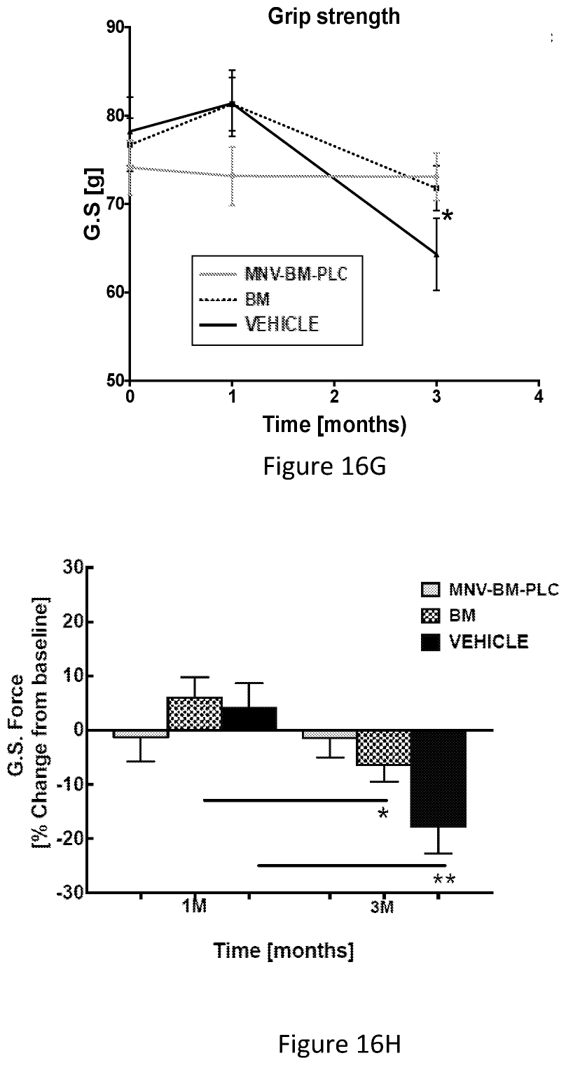

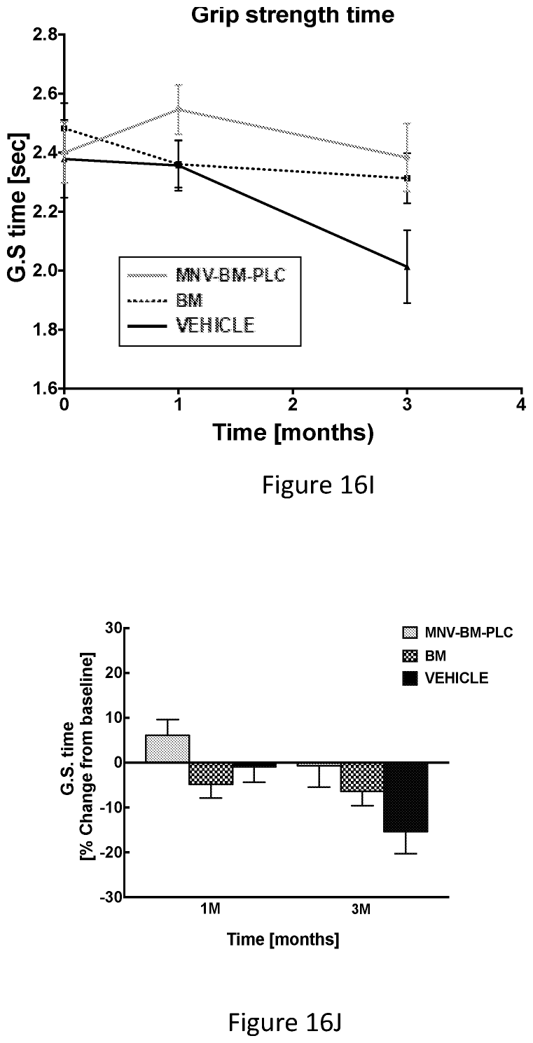

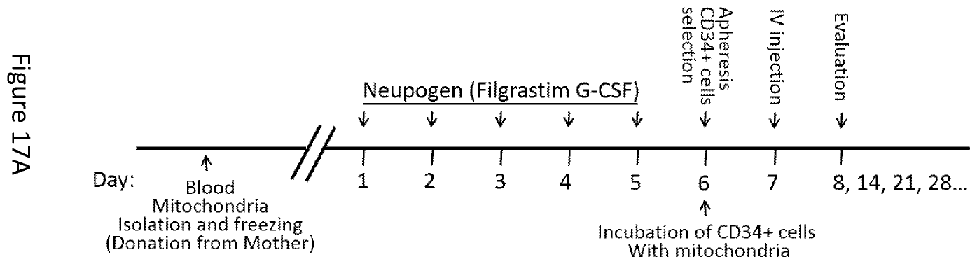

As can be seen in FIGS. 16A-16D, aging mice (12 months) transplanted with bone marrow cells enriched with healthy mitochondria (MNV-BM-PLC) demonstrated improved physical activity and exploratory behavior compared to age matched mice transplanted with bone marrow not enriched with mitochondria (BM control) and to mice not transplanted at all (control). MNV-BM-PLC treated mice showed: greater distance moved (FIG. 16A), spending more time in the center (FIG. 16B) and less time next to the walls (FIG. 16C) of the cage, compared to their controls, typical behavioral pattern of younger mice. Also, administrating bone marrow enriched with functional mitochondria to aging mice arrested kidney deterioration, as portrayed in FIG. 16D.

The increase in time spent in the central zone of the arena indicates an extensive exploratory behavior of mice that underwent mitochondrial augmentation therapy. Along with the reduction in thigmotaxis, which is associated with anxiety-like behaviors, it attests to an anxiolytic effect of mitochondrial augmentation.

Gross motor performance and coordination were also assessed, using a Rotarod device in these mice.

As shown in FIGS. 16E-16F, 1 month post administration, VEHICLE and BM control groups showed a decrease in latency to fall off the rotating rod (−2.82% and −2.18% from baseline, ns) which further declined by 14.15% and 21.79% (***p=0.0008) relative to baseline 3 months post administration. MNV-BM-PLC mice exhibited a 16.17% reduction in latency to fall off the rod 1 month post mitochondria enrichment therapy (*p=0.0464), halted 3 months post enrichment (−8.72% from baseline, ns).

The results demonstrate more moderate motor function impairment in mitochondria-enriched middle-aged mice relative to age-matched controls, implying that mitochondrial enrichment therapy can attenuate age-related motor function deterioration.

Skeletal muscle function was also evaluated by the forelimb grip strength test in these mice. As shown in FIGS. 16G-16H, MNV-BM-PLC mice maintained their grip strength score constant at 1 month and 3 months post mitochondria augmentation (enrichment) therapy (−1.29% and −1.40% of baseline, respectively, and exhibited a slower deterioration in grip strength time (latency to release grip) starting 3 months post administration (+6.07% and −0.69% of baseline 1 and 3 months post administration.

As shown in FIGS. 16I-16J compared with VEHICLE and BM control groups, in which a −4.80% and −0.9% decline from baseline observed 1 month post administration further aggravated 2 months later (−15.3% and −6.35% of baseline, ns, respectively). VEHICLE and BM control mice' baseline grip strengths were increased 1 month post administration (+6.01% and +4.06% from baseline, ns), declining by 2 months later to −6.03% (**p=0.0084) and −17.77% (*p=0.0404) of baseline, respectively.

These results show a slower/reduced deterioration in grip strength and retention time in mitochondria-enriched treated mice suggest that mitochondria enrichment therapy may ameliorate age-related impairment in muscle function.

### Example 13. Diminishing the Debilitating Effects of Aging and Age-Related Disease in Human Subjects

The steps of the method for diminishing debilitating effects in aging human subjects or subjects afflicted with age-related disease or diseases are: (1) administering to the aging subject or donor G-CSF in a dosage of 10-16 μg/kg for 5 days; (2) on day 5, consider administering to the subject Mozobil, for 1-2 days; (3) on day 6, performing apheresis on the blood of the subject to obtain bone marrow cells. If the stem cells amount is insufficient, apheresis can be performed again on day 7; (4) in parallel, isolating functional mitochondria from a blood sample or placenta of a healthy donor. The isolation of the functional mitochondria can also be performed prior to this process, storing the mitochondria frozen at −80° C. (at least) and defrosted prior to use; (5) incubation of bone marrow cells with functional mitochondria for 24 hours; (6) washing the bone marrow cells; and (7) infusion of bone marrow cells enriched with mitochondria to the aging subject. During the entire period, evaluating changes in the patient's food consumption, body weight, lactic acidosis, blood counts and biochemical blood markers.

Another method for diminishing debilitating effects of aging human subjects or subjects afflicted with age-related disease or diseases are: (1) obtaining fat tissue of the aging subject using a surgical procedure such as liposuction; (2) isolating mesenchymal stem cells (MSCs), propagating the cells in culture, and optionally cryopreservation of the cells; (3) in parallel, isolating functional mitochondria from a blood sample or placenta of a healthy donor. The isolation of the functional mitochondria can also be performed prior to this process, storing the mitochondria frozen at −80° C. (at least) and defrosted prior to use; (5) incubation of MSCs with functional mitochondria for 24 hours; (6) washing the MSCs; and (7) infusion of MSCs enriched with mitochondria to the subject. During the entire period, evaluating changes in the patient's food consumption, body weight, lactic acidosis, blood counts and biochemical blood markers.

### Example 14. Therapy of Human Patients Afflicted by a Non-Hematopoietic Neoplastic Disease

The steps of the method for therapy of human patients afflicted by a non-hematopoietic neoplastic disease are (1) administering to a patient afflicted by a neoplastic disease, G-CSF in a dosage of 10-16 μg/kg for 5 days; (2) on day 6, performing apheresis on the blood of the patient to obtain bone marrow cells; (3) in parallel, isolating functional mitochondria from a blood sample of a healthy donor; (4) incubation of bone marrow cells with functional mitochondria for 24 hours; (5) washing the bone marrow cells; and (6) infusion of bone marrow cells loaded with mitochondria to the patient. During the entire period, evaluating changes in the patient's food consumption, body weight, lactic acidosis, blood counts and biochemical blood markers.

### Example 15. Compassionate Treatment Using Autologous CD34⁺ Cells Enriched with MNV-BLD (Blood Derived Mitochondria) for a Young Patient with Pearson Syndrome (PS)

A 6.5-years old male patient (patient 1) was diagnosed with Pearson Syndrome, having a deletion of nucleotides 5835-9753 in his mtDNA. Prior to mitochondrial augmentation therapy (MAT), his weight was 14.5 KG, he was not able to walk more than 100 meters or to climb stairs. His growth was significantly delayed for 3 years prior to treatment, and at baseline his weight was −4.1 standard deviation score (SDS) and height −3.2 SDS (relative to the population), with no improvement despite being fed by a gastrostomy tube (G-tube) for more than a year. He had renal failure (GFR 22 ml/min) and proximal tubulopathy requiring electrolyte supplementation. He had hypoparathyroidism requiring calcium supplementation, and an incomplete right bundle branch block (ICRBB) on electrocardiography.

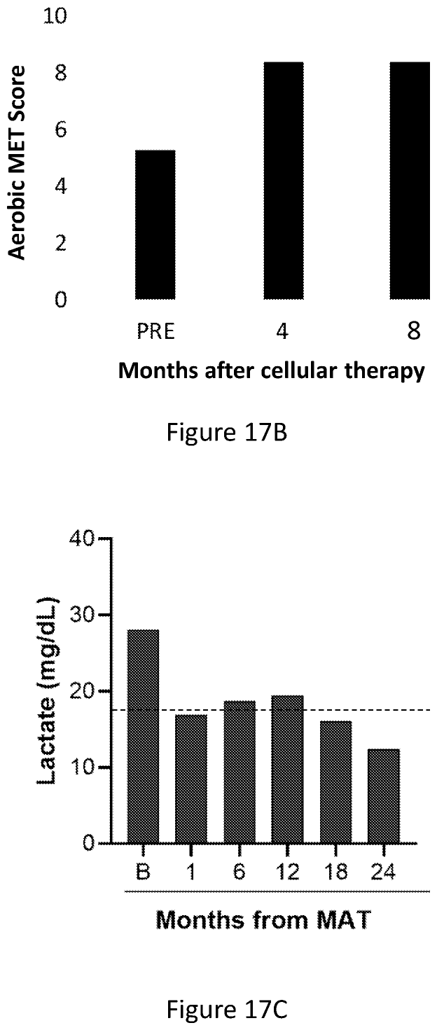

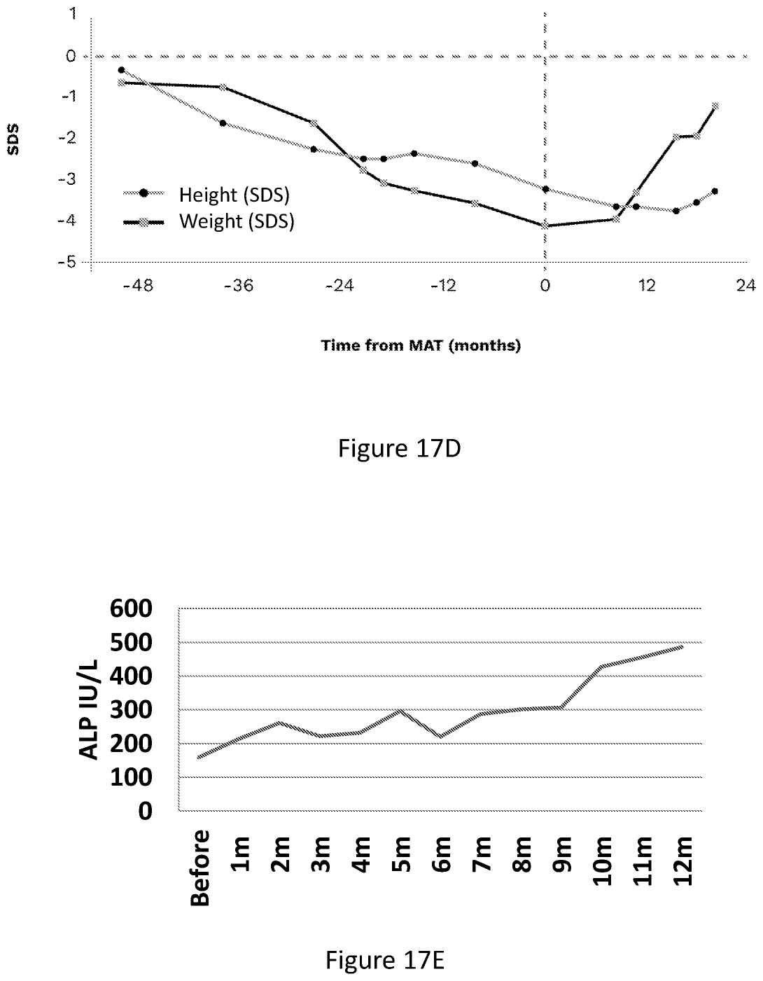

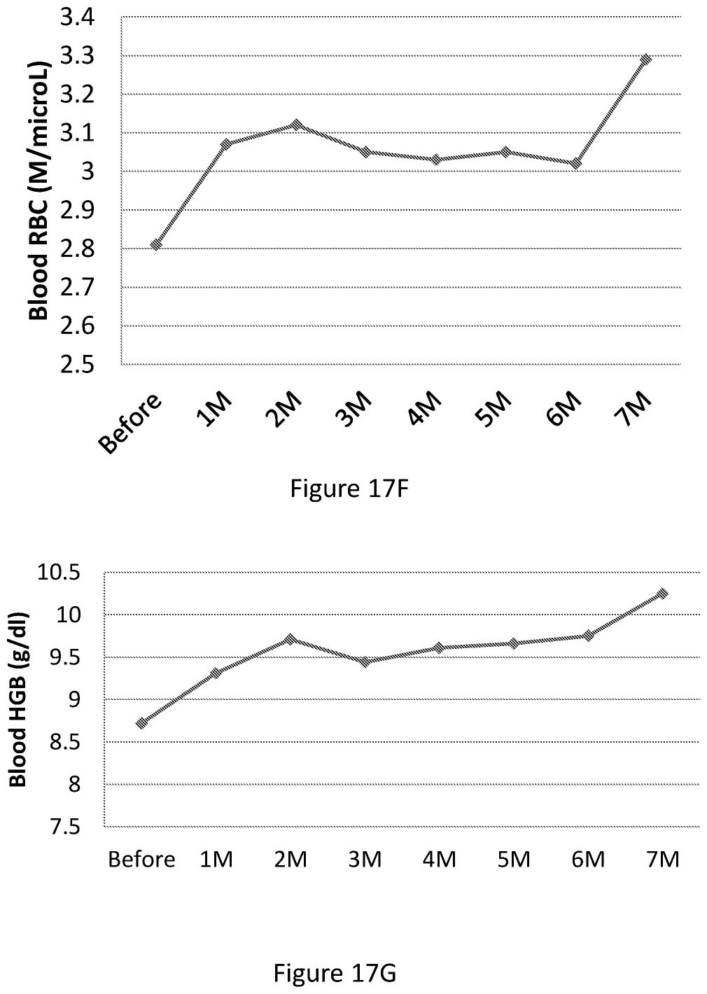

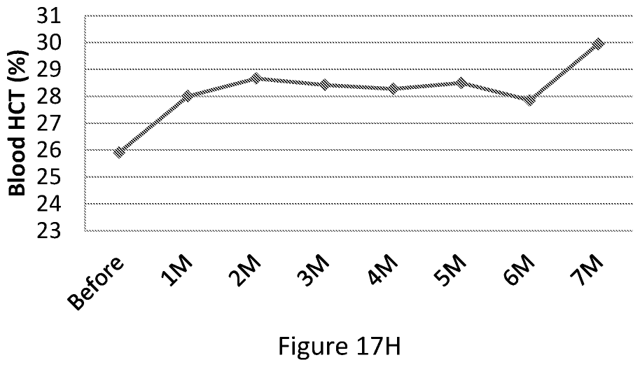

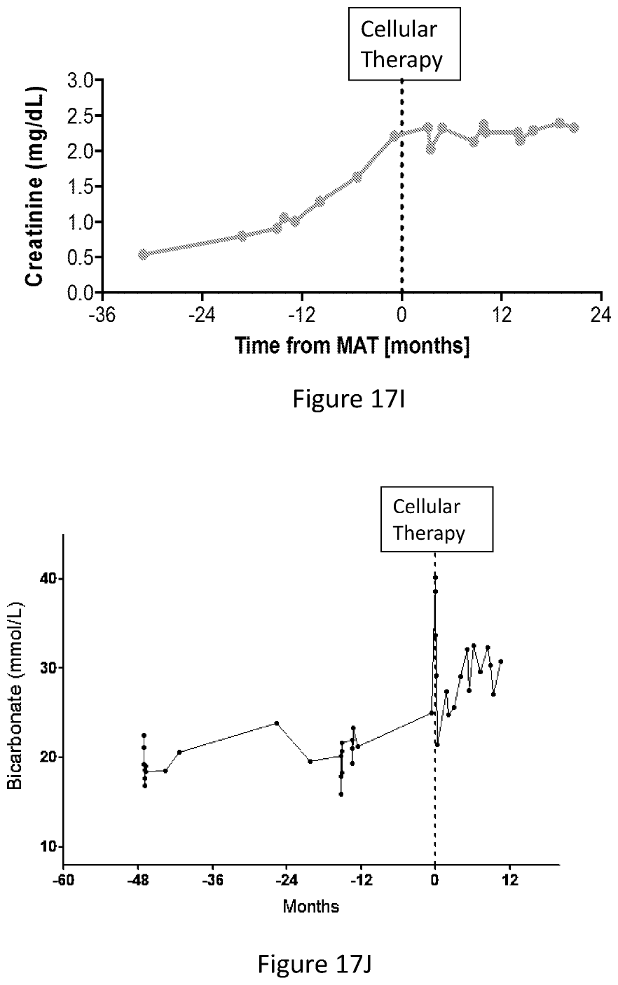

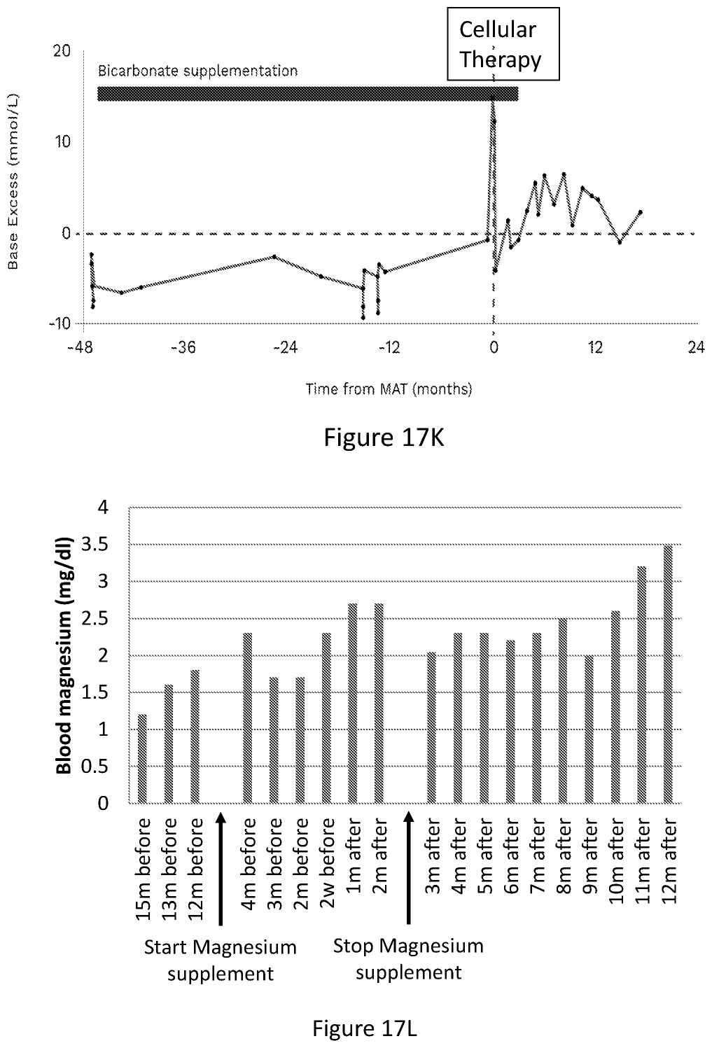

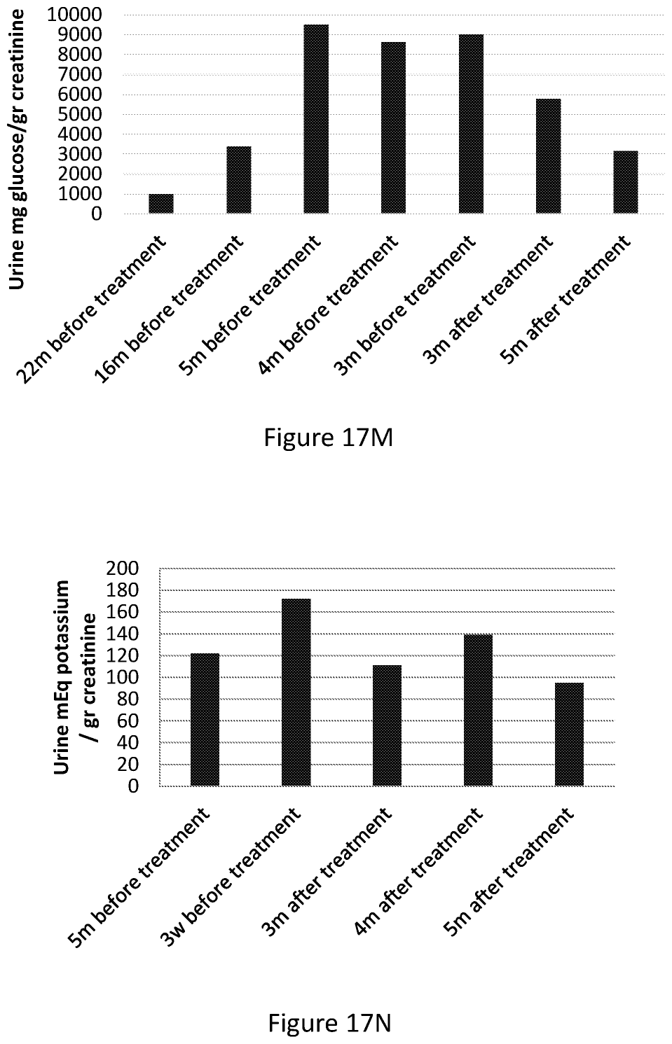

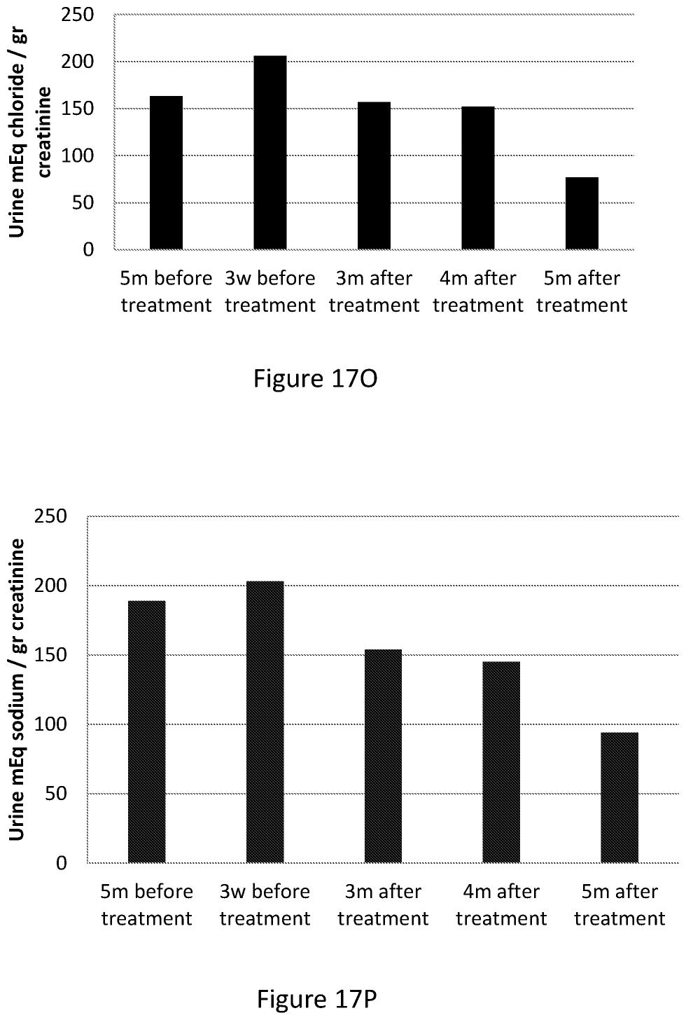

Mobilization of hematopoietic stem and progenitor cells (HSPC) was performed by subcutaneous administration of GCSF (10 μg/kg), given alone for 5 days. Leukapheresis was performed (n=2) using a Spectra Optia system (TerumoBCT), via peripheral vein access, according to institutional guidelines. CD34 positive selection was performed on mobilized peripheral blood derived cells by using the CliniMACS CD34 reagent according to the manufacturer's instructions. Mitochondria were isolated from maternal peripheral blood mononuclear cells (PBMCs) using 250 mM sucrose buffer pH 7.4 by differential centrifugation. For mitochondrial augmentation therapy (MAT), the autologous CD34⁺ cells were incubated with the healthy mitochondria from the patient's mother (1*10⁶ cells per amount of mitochondria having 4.4 units of citrate synthase (CS)), resulting in a 1.56 fold increase in the cells' mitochondrial content (56% increase in mitochondrial content as demonstrated by CS activity). Incubation with mitochondria was performed for 24 hours at RT in saline containing 4.5% HSA. Enriched cells were suspended in 4.5% human serum albumin in saline solution. The patient received a single round of treatment, by IV infusion, of 1.1*10⁶ autologous CD34⁺ cells enriched with healthy mitochondria per kilogram body weight, according to the timeline presented in FIG. 17A.

As can be seen in FIG. 17B, the aerobic Metabolic Equivalent of Task (MET) score of the patient was increased 4 months after the transplantation of mitochondrially enriched cells, an effect that remained unchanged 8 months after transplantation. The data teach that the aerobic MET score of the patient was significantly increased post-therapy over time, from 5 (moderate intensity activities, such as walking and bicycling) to 8 (vigorous intensity activities, such as running, jogging and rope jumping). The MET is a physiological measure expressing the energy cost of physical activities. The ability of enriched cells transplantation to improve this parameter is encouraging for aging subjects, since the aerobic MET score declines with age.

FIG. 17C presents the level of lactate found in the blood of the patient as a function of time post the I.V. injection. Blood lactate is lactic acid that appears in the blood as a result of anaerobic metabolism when mitochondria are damaged or when oxygen delivery to the tissues is insufficient to support normal metabolic demands, one of the hallmarks of mitochondria dysfunction. As can be seen in FIG. 4C, after MAT, blood lactate level of patient 1 has decreased to normal values. Lactate is oxidized in the mitochondria, which is partially responsible for lactate turnover in the human body. As mitochondrial quality and activity declines with age, the lactate levels rise. Therefore, the ability of enriched bone marrow stem cells to lower lactate levels implies a potential effect on the aging subject.

Table 5 presents the Pediatric Mitochondrial Disease Scale (IPMDS)—Quality of Life (QoL) Questionnaire results of the patient as a function of time post cellular therapy. In both the “Complaints & Symptoms” and the “Physical Examination” categories, 0 represents “normal” to the relevant attribute, while aggravated conditions are scored as 1-5, dependent on severity.

TABLE 5

| | Pre-treatment | +6 months |
|---|---|---|
| Complaints & Symptoms | 24 | 11 |
| Physical Examination | 13.4 | 4.6 |

It should be noted that the patient has not gained weight in the 3 years before treatment, i.e. did not gain any weight since being 3.5 years old. The data presented in FIG. 17D shows the growth measured by standard deviation score of the weight and height of the patient, with data starting 4 years prior to MAT and during the follow-up period. The data indicates that approximately 15 months following a single treatment, there was an increase in height and weight in this patient.

Another evidence for the patient's growth comes from his Alkaline Phosphatase levels. An alkaline phosphatase level test (ALP test) measures the amount of alkaline phosphatase enzyme in the bloodstream. Having lower than normal ALP levels in the blood can indicate malnutrition, which could be caused by a deficiency in certain vitamins and minerals. The data presented in FIG. 17E indicates that a single treatment was sufficient to elevate the Alkaline Phosphatase levels of the patient from 159 to 486 IU/L in only 12 months. The trend reversal of weight loss as well as the ALP elevation are relevant to both aging and anti-cancer treatments, which may lead to weight loss and malnutrition.

As can be seen in FIGS. 17F-H, treatment resulted in pronounced improvements in red blood cells levels (FIG. 17F), hemoglobin levels (FIG. 17G) and hematocrit levels (FIG. 17H). These results show that a single treatment was sufficient to ameliorate symptoms of anemia

FIG. 17I demonstrates the arrest in kidney deterioration, as depicted by urine creatinine levels post cellular transplantation. As can further be seen in FIGS. 17J and 17K, cellular treatment also resulted in pronounced improvements in the levels of bicarbonate (FIG. 17J) and base excess (FIG. 17K) without supplementing with bicarbonate. FIG. 17L presents the level of magnesium in the blood of the patient as a function of magnesium supplementation and time post cellular therapy. The data teach that the blood level of magnesium of the patient was significantly increased over time, such that magnesium supplementation was no longer required. Attaining high levels of magnesium, without magnesium supplementation, is evidence of improved magnesium absorption as well as re-absorption in the kidney proximal tubule. As can be seen in FIGS. 17M-17P, a single treatment also resulted in pronounced reduction in the levels of several renal tubulopathy indicators, such as glucose levels (FIG. 17M) and certain salt levels in the urine (FIG. 17N—potassium; FIG. 17O—chloride; FIG. 17P—sodium). FIGS. 17I-17P are all relevant to the aging subject, as kidney function deteriorates with age.

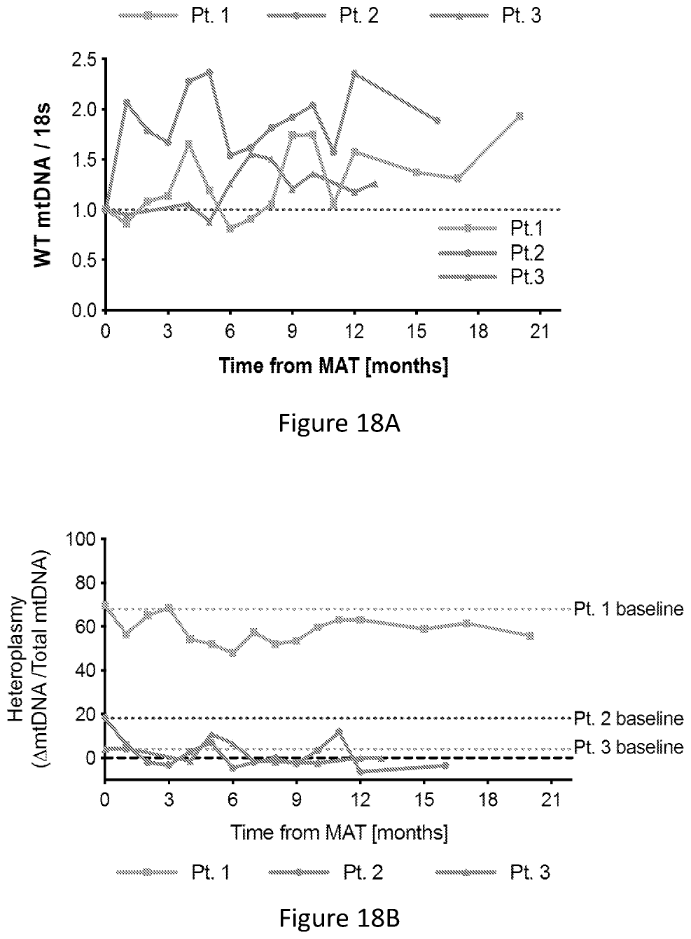

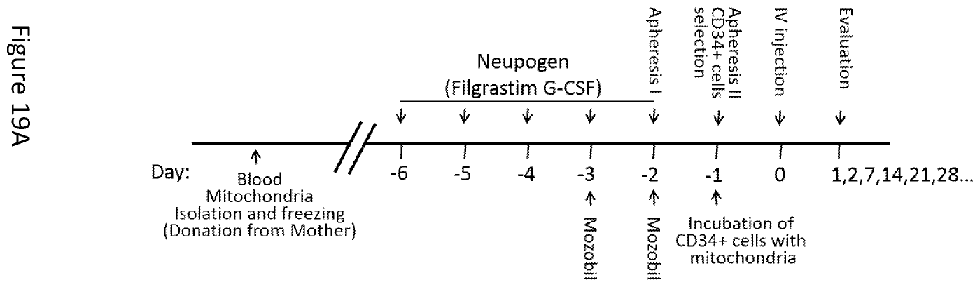

A genetic indication to the success of the therapy used is the prevalence of normal mtDNA compared to total mtDNA per cell. As illustrated in FIG. 18A (Pt.1), the prevalence of total normal mtDNA in the peripheral blood of the patient was increased from a baseline of about 1 to as high as 1.6 (+60%) in just 4 months, and to 1.9 (+90%) after 20 months from treatment, and above the baseline level in most of the time points. Notably, normal mtDNA levels were above the baseline level on most of the time points.

Another indication for the effectiveness of transplanting cells enriched with healthy functional mitochondria is presented in FIG. 18B. There is a slight decrease in heteroplasmy (less deleted mtDNA) following MAT in patient 1 who had relatively high levels of heteroplasmy at baseline. This was ongoing throughout the follow-up period.

According to a Hospital's neurologist report, neurological improvement has been demonstrated after transplantation of autologous cells with healthy mitochondria not carrying the deletion mutation; the patient improved his walking skills, climbing steps, using scissors and drawing. Substantial improvements were noted in executing commands and response time as well as in motor and language skills. Also, the mother reported an improvement in memory. These findings are particularly relevant and important for the aging subject, since neurological deterioration in motor skills and memory often occurs in old age.

As the data presented above indicates, a single round of the therapeutic method of administering bone marrow stem cells enriched with functional mitochondria was successful in treating numerous debilitating conditions afflicted by aging.

### Example 16. Compassionate Treatment Using Autologous CD34⁺ Cells Enriched with MNV-BLD (Blood Derived Mitochondria) for a Juvenile with Pearson Syndrome (PS)

A 7-years old female patient (patient 2) was diagnosed with Pearson Syndrome, having a deletion of 4977 nucleotides in her mtDNA. The patient also suffers from anemia, endocrine pancreatic insufficiency, and is diabetic (HbAlC 7.1%). Patient 2 has high lactate levels (>25 mg/dL), low body weight, and problems with eating and gaining weight. The patient further suffers from hypermagnesuria (high levels of magnesium in urine, low levels in blood). Patient has memory and learning problems, astigmatism, and low mitochondrial activity in peripheral lymphocytes as determined by TMRE, ATP content and O₂ consumption rate (relative to the healthy mother).

Mobilization of bone marrow was done using G-CSF (10 μg/kg) and 1 dose of Plerixafor Mozobil™ (0.24 mg/ml). Patient began treatment with 1.8*10⁶ cells/kg autologous CD34⁺ cells enriched with healthy mitochondria isolated from her mother, according to the timeline presented in mobilization of HSPC, leukapheresis and CD34 positive selection were performed similar to patient 1 (Example 18) with the addition of plerixafor (n=2) administration 1 day prior to leukapheresis. Mitochondria were isolated from maternal peripheral blood mononuclear cells (PBMCs) using 250 mM sucrose buffer pH 7.4 by differential centrifugation. For MAT, the autologous CD34⁺ cells were incubated with the healthy mitochondria from the patient's mother (10⁶ cells per amount of mitochondria having 4.4 units of citrate synthase (CS)), resulting in a 1.62 fold increase in the cells mitochondrial content (62% increase in mitochondrial content as demonstrated by CS activity). Incubation with mitochondria was performed for 24 hours at RT in saline containing 4.5% HSA. It should be noted that after mitochondrial enrichment, the CD34⁺ cells from the patient increased the rate of colony formation by 26%.

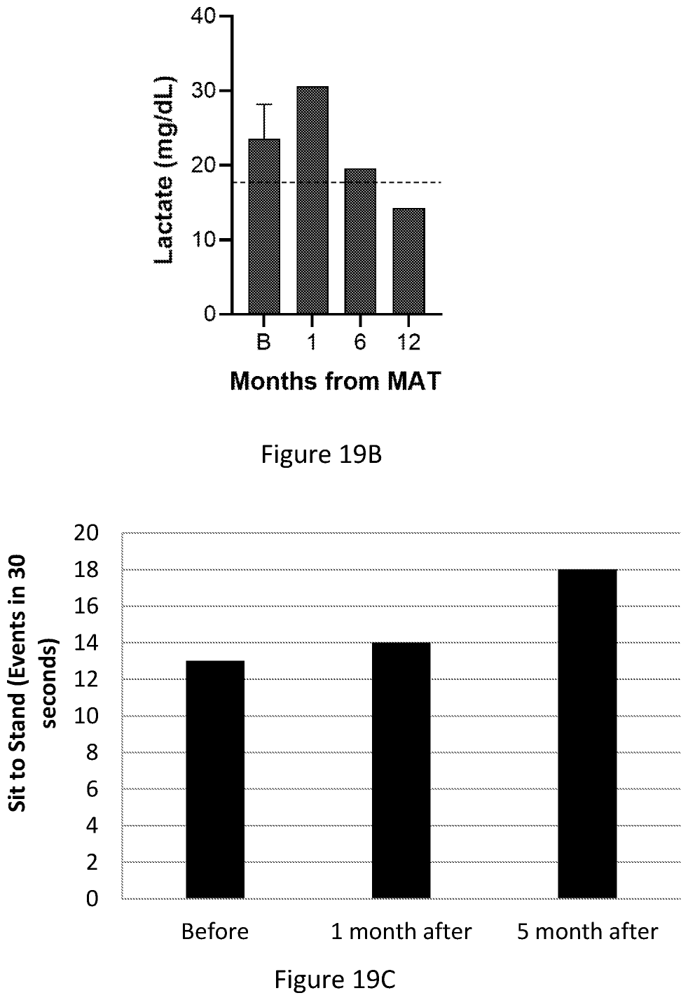

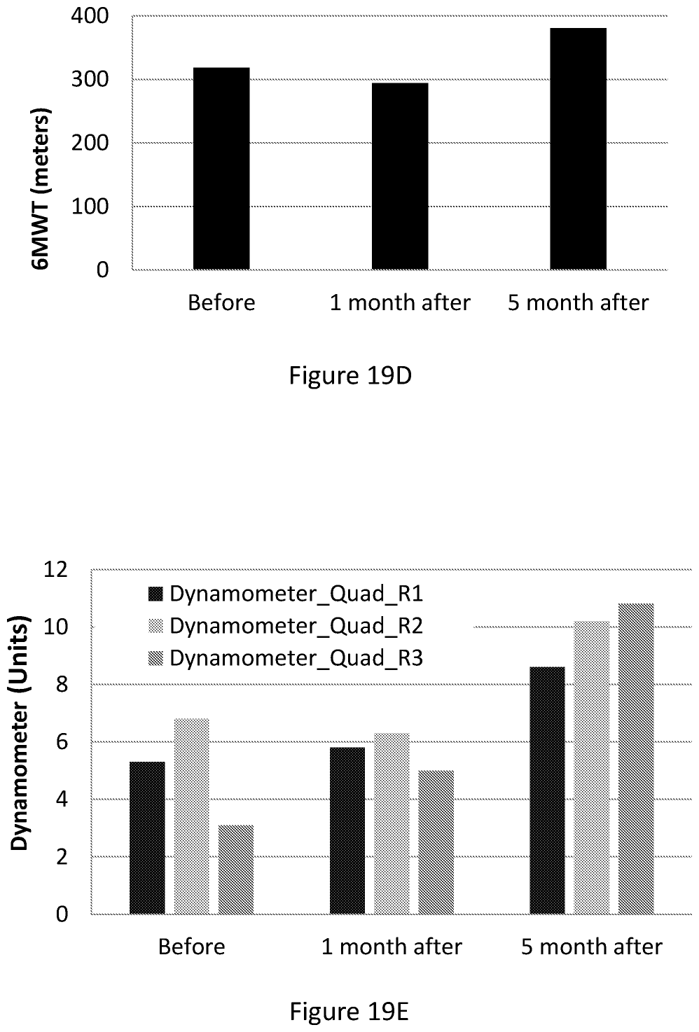

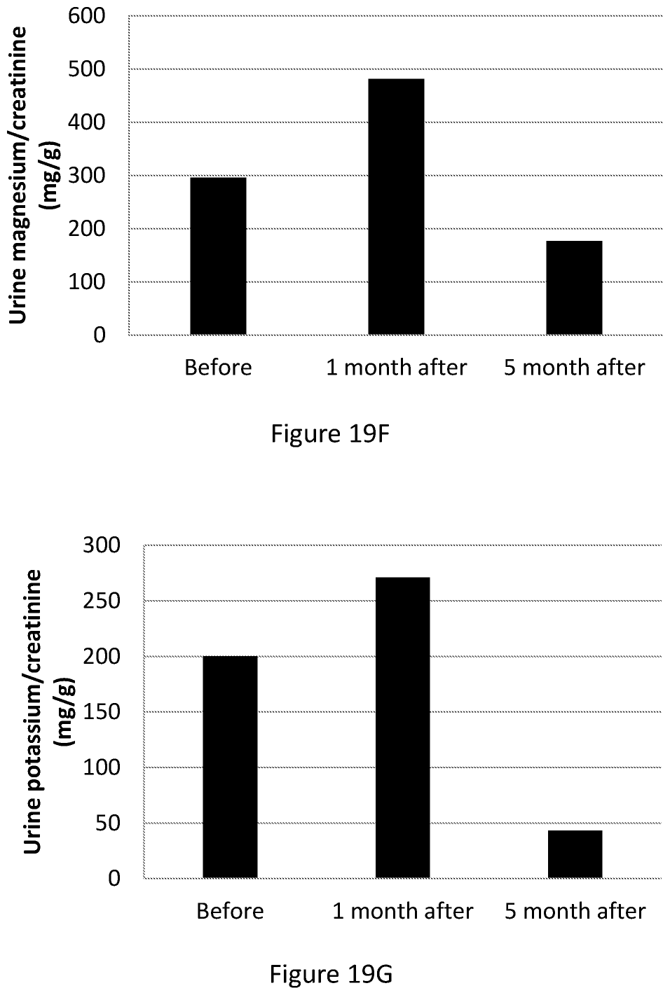

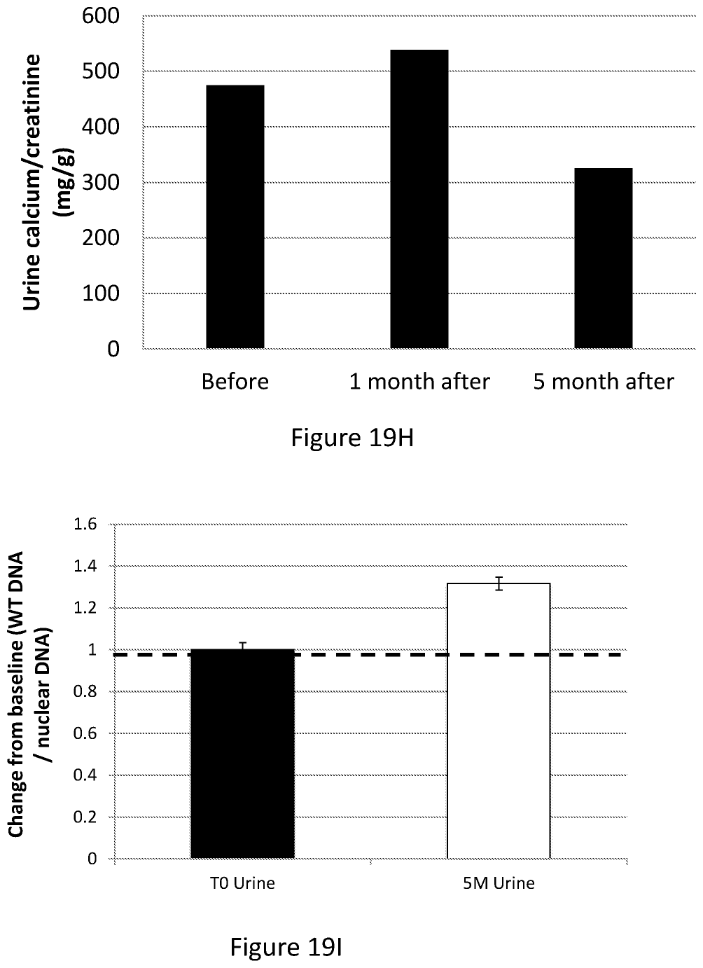

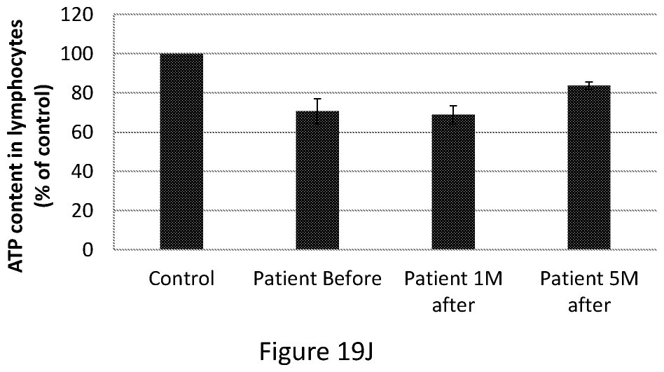

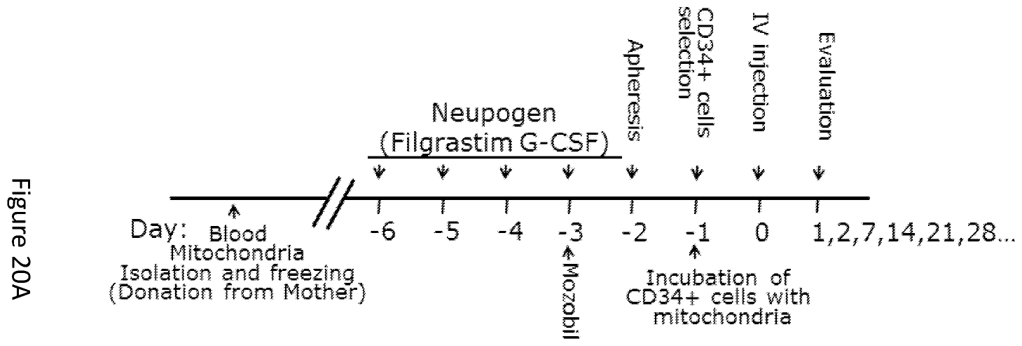

Patient 2 (15 KG at day of treatment) was treated, by IV infusion, with 1.8*10⁶ autologous CD34⁺ cells enriched with healthy mitochondria per kilogram body weight, according to the timeline presented in FIG. 19A.

FIG. 19B portrays the beneficial effect of mitochondrially enriched cells transplantation on blood lactate levels, which is decreased 5 months after treatment.

Muscle strength and mass are known to deteriorate with aging. FIGS. 19C-19E demonstrate the remarkable effect of the transplantation of enriched cells on these parameters in a series of functional tests. FIG. 19C shows sit to stand test results. Elderly who are unable to stand up from a chair without support are at risk of becoming more inactive and thus of further mobility impairment. The tested subjects are invited to perform as many sit to stand cycles as possible within a timeframe of 30 seconds. Patient 2 was able to perform more sit to stand cycles 5 months post transplantation. FIG. 19D portrays a 6 minute walk test (6MWT) and measures the distance in meters the subject has passed within the allocated 6 minutes. Patient 2 passed a normal distance 5 months after transplantation. FIG. 19E shows improvement in muscle strength 5 months after cell transplantation, as evident from the elevated dynamometer units, even after the 3rd consecutive repeat against the resistance of the dynamometer.

FIGS. 19F, 19G and 19H present the improved kidney function illustrated by ratios of magnesium, potassium and calcium compared to creatinine found in the urine of the patient as a function of time post the I.V. injection, respectively.

FIG. 19I presents the ratio between ATP8 to 18S in the urine of the patient as a function of time post the I.V. injection. The immune system is deteriorating with age. Amongst the immune system components most affected by aging are T lymphocytes. In the young, naïve T cells can metabolize glucose, amino acids, and lipids to catabolically fuel ATP generation in the mitochondria. Since mitochondrial function is also known to be compromised with aging, a possible connection between T cells and mitochondrial decline has been suggested and is being studied. FIG. 19J shows an increase in ATP content in lymphocytes of FIG. 18A (Pt.2) presents the prevalence of normal mtDNA as a function of time post the I.V. injection. As can be seen in FIG. 6B (Pt.2), the prevalence of normal mtDNA was increased from a baseline of about 1 to as high as 2 (+100%) in just 1 month, remaining relatively high until 10 months post treatment. Notably, normal mtDNA levels were above the baseline level on all the time points

FIG. 18B (Pt.2) presents the change in heteroplasmy level as a function of time after MAT. It can be seen that there was a decrease in heteroplasmy (less deleted mtDNA) following MAT in patient 2. This was ongoing throughout the follow-up period.

### Example 17. Compassionate Treatment Using Autologous CD34⁺ Cells Enriched with MNV-BLD (Blood Derived Mitochondria) for a Young Patient with Pearson Syndrome (PS) and PS-Related Fanconi Syndrome (FS)

A 10.5-years old female patient (patient 3) was diagnosed with Pearson Syndrome, having a deletion of nucleotides 12113-14421 in her mtDNA. The patient also suffers from anemia, and from Fanconi Syndrome that developed into kidney insufficiency stage 4. Patient is treated with dialysis three times a week. Recently, the patient also suffers from a severe vision disorder, narrowing of the vision field and loss of near vision. Patient is incapable of any physical activity at all (no walking, sits in a stroller)

Patient had high lactate levels (>50 mg/dL), and a pancreatic disorder which was treated with insulin. Brain MRI showed many lesions and atrophic regions. Patient was fed only through a gastrostomy. Patient had memory and learning problems. Patient had low mitochondrial activity in peripheral lymphocytes as determined by Tetramethylrhodamine Ethyl Ester (TMRE), ATP content and O₂ consumption rate (relative to the healthy mother) tests.

Mobilization of hematopoietic stem and progenitor cells (HSPC) as well as leukapheresis and CD34 positive selection were performed similar to patient 1 (Example 3) with the addition of plerixafor (n=1) on day −1 prior to leukapheresis. Leukapheresis was performed via a permanent dialysis catheter. Mitochondria were isolated from maternal peripheral blood mononuclear cells (PBMCs) using 250 mM sucrose buffer pH 7.4 by differential centrifugation. For MAT, the autologous CD34⁺ cells were incubated with healthy mitochondria from the patient's mother (1*10⁶ cells per amount of mitochondria having 4.4 units of citrate synthase (CS)), resulting in a 1.14 fold increase in the cells mitochondrial content (14% increase in mitochondrial content as demonstrated by CS activity). Cells were incubated with mitochondria for 24 hours at R.T. in saline containing 4.5% HSA. It should be noted that after mitochondrial enrichment, the CD34⁺ cells from the patient increased the rate of colony formation by 52%.

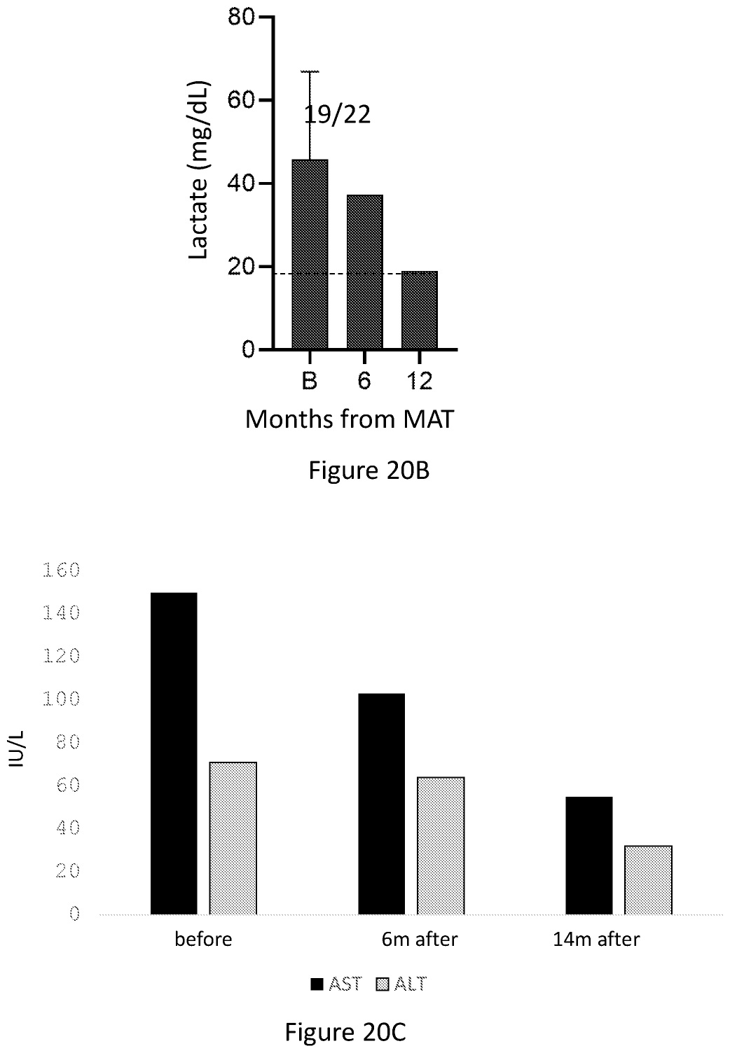

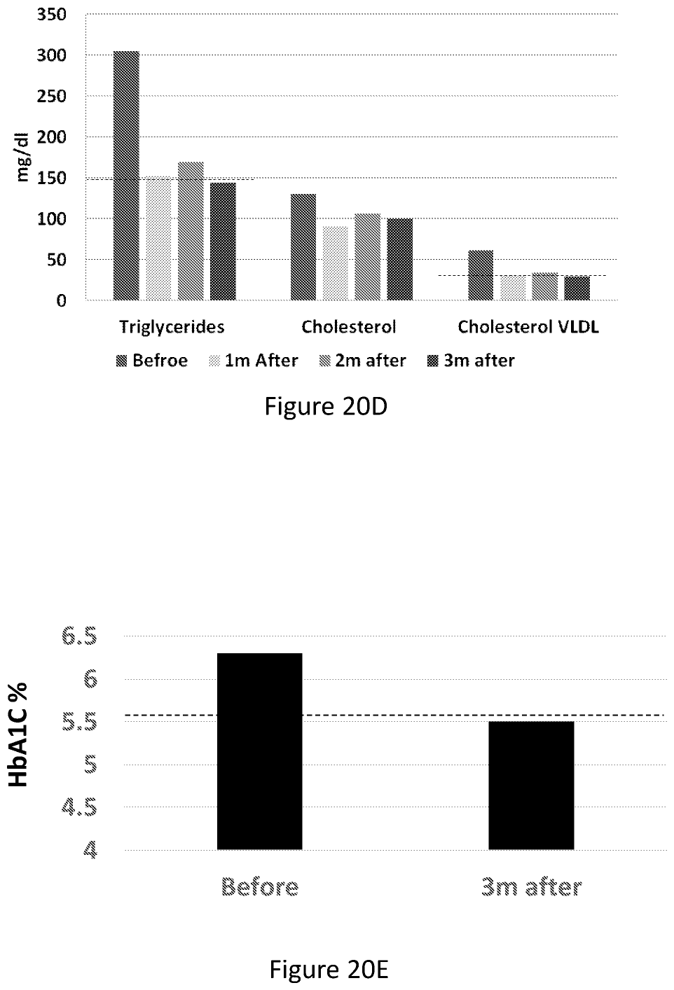

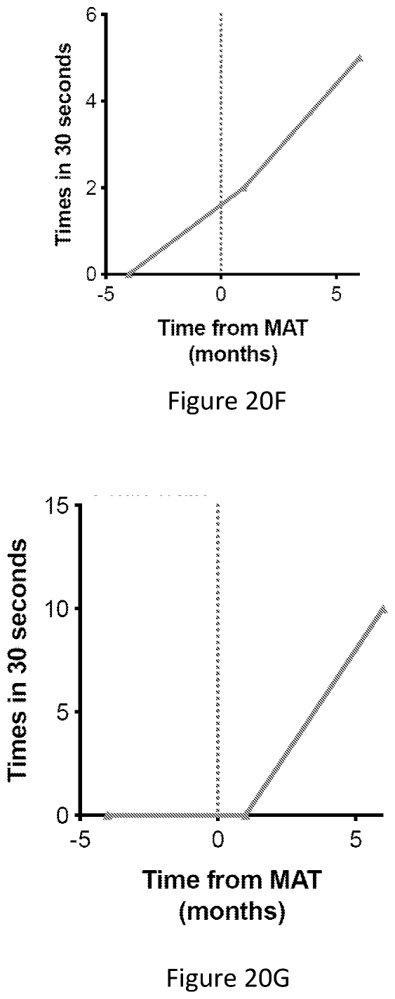

Patient 3 (21 KG) was treated, by IV infusion, with 2.8*10⁶ autologous CD34⁺ cells enriched with healthy mitochondria from her mother per kilogram body weight, according to the timeline presented in FIG. 20A.

FIG. 202B portrays the beneficial effect of mitochondrially enriched cells transplantation on blood lactate levels, which are decreased 2 and 3 months after transplant. The line below 20 mg/dl represents blood lactate normal levels.

FIG. 20C presents the levels of AST and ALT liver enzymes in the blood of the patient as a function of time before and after cellular therapy. Attaining low levels of liver enzymes in the blood is evidence of decreased liver damage.

FIG. 20D presents the levels of triglycerides, total cholesterol and very-low-density lipoprotein (VLDL) cholesterol in the blood of the patient as a function of time before and after cellular therapy. Attaining low levels of triglycerides, total cholesterol and VLDL cholesterol in the blood is evidence of increased liver function and improved lipid metabolism.

Glycated hemoglobin (sometimes also referred to as hemoglobin A1c, HbA1c, A1C, Hb1c, Hb1c or HGBA1C) is a form of hemoglobin that is measured primarily to identify the three-month average plasma glucose concentration. The test is limited to a three-month average because the lifespan of a red blood cell is four months (120 days). FIG. 20E presents the result of the A1C test of the patient as a function of time before and after therapy.

FIGS. 20F and 20G present the results of the “Sit-to-Stand” (20F) and “6-minute-walk” (20G) tests of the patient as a function of time post the I.V. injection, showing an improvement in both parameters 5 months after treatment.

FIG. 10A (Pt.3) presents the prevalence of normal mtDNA as a function of time post the I.V. injection. As can be seen in FIG. 10A (Pt.3), the prevalence of normal mtDNA was increased by 50% at 7 months post treatment. Notably, normal mtDNA levels were above the baseline level on most of the time points

FIG. 10B (Pt.3) presents the change in heteroplasmy level as a function of time after MAT. It can be seen that there was a decrease in heteroplasmy (less deleted mtDNA) following MAT in patient 3 who had relatively low levels of heteroplasmy at baseline. This was ongoing throughout the follow-up period.

### Example 18. Compassionate Treatment Using Autologous CD34⁺ Cells Enriched with MNV-BLD (Blood Derived Mitochondria) for a Juvenile with Kearns-Sayre Syndrome (KSS)

Patient 4 was a 14-years old, 19.5 kg female patient, diagnosed with Kearns-Sayre syndrome, experiencing tunnel vision, ptosis, ophthalmoplegia and retinal atrophy. The patient had vision problems, CPEO, epileptic seizures, pathologic EEG, sever myopathy with disability to sit or walk, cardiac arrhythmia. The patient had a 7.4 Kb deletion in her mitochondrial DNA, including the following genes: TK, NCB, ATP8, ATP6, CO3, TG, ND3, TR, ND4L, TH, TS2, TL2, ND8, ND6, TE, NC9 and CYB.

Mobilization of hematopoietic stem and progenitor cells (HSPC) as well as leukapheresis and CD34 positive selection were performed similar to patient 3 (Example 5). For MAT, the autologous CD34⁺ cells were incubated for 24 hours at R.T. with healthy mitochondria from the patient's mother (1*10⁶ cells per amount of mitochondria having 4.4 units of citrate synthase (CS)), in saline containing 4.5% HSA. The enrichment resulted in a 1.03 fold increase in the cells mitochondrial content (3% increase in mitochondrial content as demonstrated by CS activity).

Patient 4 was treated with 2.2*10⁶ autologous CD34⁺ cells enriched with healthy mitochondria per kilogram body weight, according to the timeline presented in FIG. 20A.

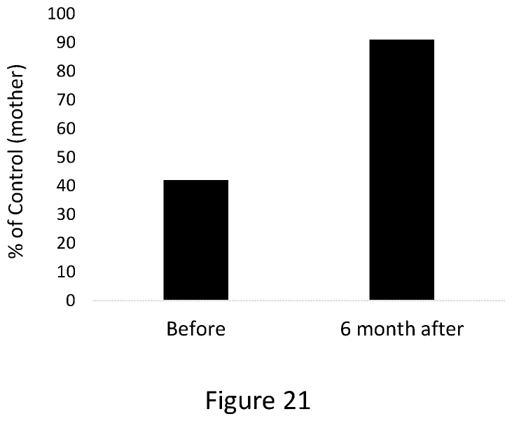

Unexpectedly, 4 months after a single treatment with CD34⁺ that were enriched by only 3% with healthy mitochondria, the patient showed improvement in EEG and no epileptic seizures. Five months after treatment the patient suffered disease-related atrioventricular (AV) block and a pacer was installed. The patient recovered and improvement continued. The ATP content in the peripheral blood was measured 6 months post-treatment, showing an increase of about 100% in ATP content compared to that before treatment, as shown in FIG. 21. Seven months after treatment, the patient could sit by herself, walk with assistance, talk, has better appetite and gained 3.6 KG.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1.-3. (canceled)

4. The method of claim 49, wherein the anti-cancer treatments are selected from the group consisting of radiation, chemotherapy and immunotherapy with monoclonal antibodies.

5. The method of claim 49, wherein the stem cells are autologous, syngeneic or from a donor.

6. The method of claim 49, wherein the stem cells are pluripotent stem cells (PSCs), induced pluripotent stem cells (iPSCs), CD34+ cells or mesenchymal stem cells.

7. (canceled)

8. The method of claim 49, wherein the stem cells are derived from adipose tissue, oral mucosa, blood, bone marrow cells or umbilical cord blood.

9. (canceled)

10. The method of claim 49, wherein the human stem cells comprise common myeloid progenitor cells, common lymphoid progenitor cells or any combination thereof.

11. (canceled)

12. (canceled)

13. The method of claim 49, wherein the mitochondria are derived from a cell or a tissue selected from the group consisting of: placenta, placental cells grown in culture and blood cells.

14.-18. (canceled)

19. The method of claim 49, wherein the route of administration of the pharmaceutical composition to a subject is selected from the group consisting of intravenous, intraarterial, intramuscular, subcutaneous, intraperitoneal, systemic parenteral administration and direct injection or administration into a tissue or an organ.

20. The method of claim 49, wherein the mitochondrially-enriched human stem cells have:
(i) an increased mitochondrial DNA content;
(ii) an increased level of citrate synthase (CS) activity;
(iii) an increased content of at least one mitochondrial protein selected from Succinate dehydrogenase complex, subunit A (SDHA) and cytochrome C oxidase (COX1);
(iv) an increased rate of 0 2 consumption;
(v) an increased rate of ATP production; or
(vi) any combination thereof,
relative to the corresponding level in the stem cells prior to mitochondrial enrichment.

21. An ex-vivo method for enriching human stem cells with exogenous mitochondria comprising:
(i) providing a first composition, comprising a plurality of isolated or partially purified human stem cells from an individual afflicted with a debilitating condition or from a donor;
(ii) providing a second composition, comprising a plurality of isolated or partially purified mitochondria obtained from a healthy donor;
(iii) contacting the human stem cells of the first composition with the mitochondria of the second composition at a ratio of 0.088-176 mU CS activity per 10⁶ stem cells; and
(iv) incubating the composition of (iii) under conditions allowing the mitochondria to enter the human stem cells thereby enriching said human stem cells with said human mitochondria;
wherein the mitochondrial content of the enriched human stem cells is detectably higher than the mitochondrial content of the human stem cells in the first composition.

22. The method of claim 21, wherein the stem cells in the first composition are obtained from an aging subject or from a donor.

23. An ex-vivo method for enriching human stem cells with exogenous mitochondria comprising:
(i) providing a first composition, comprising a plurality of isolated or partially purified human stem cells from an individual suffering from a malignant disease or from a healthy donor;
(ii) providing a second composition, comprising a plurality of isolated or partially purified human mitochondria obtained from the same individual or from a healthy donor;
(iii) contacting the human stem cells of the first composition with the human mitochondria of the second composition at a ratio of 0.088-176 mU CS activity per 10⁶ stem cells; and
(iv) incubating the composition of (iii) under conditions allowing the human mitochondria to enter the human stem cells thereby enriching said human stem cells with said human mitochondria;
wherein the mitochondrial content of the enriched human stem cells is detectably higher than the mitochondrial content of the human stem cells in the first composition.

24. The method of claim 23, wherein the stem cells in the first composition are obtained from a subject afflicted with a non-hematopoietic malignant disease, or from a healthy donor not afflicted with a malignant disease.

25. The method of claim 23, wherein the conditions allowing the exogenous mitochondria to enter the human stem cells comprise incubating the human stem cells with said healthy functional exogenous mitochondria for a time ranging from 0.5 to 30 hours, at a temperature ranging from 16 to 37° C.

26. The method of claim 25, wherein prior to incubation the method further comprises a single centrifugation of the human stem cells and the exogenous mitochondria above 2500×g.

27. The method of claim 21, wherein the stem cells are bone marrow cells.

28. The method of claim 23, wherein the mitochondria in the second composition are obtained from a subject afflicted with a malignant disease prior to anti-cancer treatments.

29. The method of claim 21, further comprising expanding the stem cells before or after enrichment with the exogenous mitochondria.

30.-32. (canceled)

33. The method of claim 21, wherein the detectable enrichment of mitochondrial content of the stem cells prior to mitochondrial enrichment or post mitochondrial enrichment is determined by assays selected from the group consisting of: (i) content of at least one mitochondrial protein selected from SDHA and COX1; (ii) activity level of citrate synthase; (iii) rate of oxygen (O₂) consumption; (iv) rate of adenosine triphosphate (ATP) production; (v) mitochondrial DNA content; and any combination thereof.

34.-35. (canceled)

36. The method of claim 21, wherein the stem cells are derived from adipose tissue, skin fibroblasts, oral mucosa, blood or umbilical cord blood.

37. The method of claim 21, wherein the stem cells are CD34⁺ cells, mesenchymal cells, pluripotent stem cells (PSCs) or induced pluripotent stem cells (iPSCs).

38. The method of claim 21, wherein the human stem cells are derived from adipose tissue, oral mucosa, blood, umbilical cord blood or bone marrow.

39. (canceled)

40. The method of claim 21, wherein the stem cells enriched with mitochondria have:
(i) an increased content of at least one mitochondrial protein selected from SDHA and COX1.
(ii) an increased rate of oxygen (O₂) consumption;
(iii) an increased activity level of citrate synthase;
(iv) an increased rate of adenosine triphosphate (ATP) production;
(v) an increased mitochondrial DNA content; or
(vi) any combination thereof.
as compared to stem cells prior to mitochondrial enrichment.

41. The method of claim 21, wherein the total amount of mitochondrial proteins in the partially purified mitochondria is between 20%-80% of the total amount of cellular proteins within the sample.

42. A plurality of human stem cells enriched with mitochondria, obtained by the method of claim 21.

43. A pharmaceutical composition comprising a plurality of human stem cells according to claim 42.

44. (canceled)

45. A method of treating a debilitating condition in a human subject in need thereof, comprising administering to the subject the pharmaceutical composition of claim 43.

46. The method of claim 45, wherein the stem cells are autologous, allogeneic or syngeneic to the subject afflicted with the debilitating condition.

47. (canceled)

48. The method of claim 46, further comprising a step of administering to the subject suffering from debilitating conditions selected from the group consisting of aging, age-related diseases and the sequellae of anti-cancer treatments, an agent which prevents, delays, minimizes or abolishes an adverse immunogenic reaction between the subject and the stem cells of the allogeneic donor.

49. A method for treating or diminishing debilitating conditions in a subject comprising administering a pharmaceutical composition comprising at least 5×10⁵ to 5×10⁹ human stem cells enriched with exogenous mitochondria to the subject, wherein the debilitating conditions are selected from the group consisting of aging, age-related diseases and the sequel of anti-cancer treatments.

50. The method of claim 23, wherein the conditions allowing the exogenous mitochondria to enter the human stem cells comprise incubating the human stem cells with said exogenous mitochondria until the mitochondrial content in the stem cells is increased in average by 1% to 45% as compared to the initial mitochondrial content in the stem cells.
